(19) United States
(12) Patent Application Publication
(10) Pub. No.: US 2007/0037781 A1
(43) Pub. Date: Feb. 15, 2007

(54) Novel combinations of medicaments for the treatment of respiratory diseases containing long-acting beta-agonists and at least one additional active ingredient

(75) Inventors: Ingo KONETZKI, Warthausen (DE); Thierry BOUYSSOU, Birkenhard (DE); Sabine PESTEL, Attenweiler (DE); Andreas SCHNAPP, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/424,558

(22) Filed: Jun. 16, 2006

(30) Foreign Application Priority Data
Jul. 1, 2005 (DE) 10 2005 030 733

(51) Int. Cl. A61K 31/56; A61K 31/538; A61K 31/517; A61K 31/498; A61K 31/4709

(52) U.S. Cl. 514/171; 514/230.5; 514/249; 514/266.2; 514/312

(57) ABSTRACT

Disclosed are medicament combinations which contain in addition to one or more, preferably one, compound of general formula 1 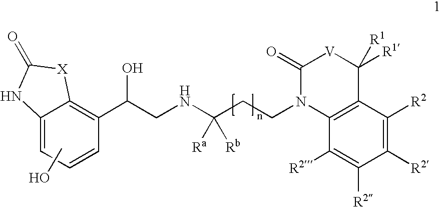
wherein the groups X, R^{a}, R^{b}, R¹, R^{1′}, R², R^{2′}, R^{2″}, R^{2′″}, V and n may have the meanings given in the claims and in the specification, at least one other active substance 2, processes for preparing them and their use as pharmaceutical compositions.

### RELATED APPLICATION DATA

This application claims benefit to DE 10 2005 030 733 filed Jul. 1, 2005.

The present invention relates to new combinations of medicaments which contain in addition to one or more, preferably one, compound of general formula 1 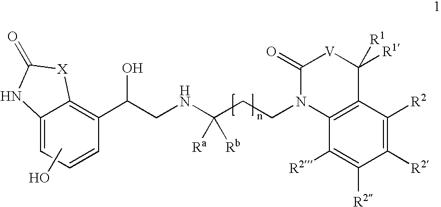
wherein the groups X, R^{a}, R^{b}, R¹, R^{1′}, R², R^{2′}, R^{2″}, R^{2′″}, V and n may have the meanings given in the claims and specification, at least one other active substance 2, processes for preparing them and their use as medicaments.

### DETAILED DESCRIPTION OF THE INVENTION

The present invention relates to medicament combinations, which contain in addition to one or more, preferably one, compound of general formula 1 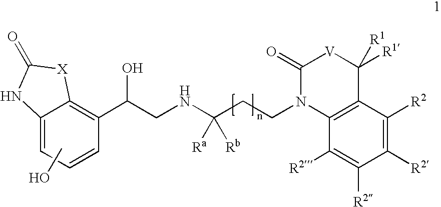
wherein
X denotes a group —O—, —NH—, —CH₂—O—, —CHMe-O—, —C(Me)₂-O—, —CH₂—NH—, —CHMe-NH—, —C(Me)₂-NH—, —CH═CH— or —CH₂—CH₂—;
V denotes a double-bonded group selected from among CH₂, NH and O, preferably CH₂ and O, particularly preferably O;
R^{a} and R^{b} which are identical or different, denote a group selected from among hydrogen, C₁₋₄-alkyl and halogen-C₁₋₄-alkyl,
   or
R^{a} and R^{b} together denote a C₂₋₅-alkylene bridge, wherein one or more hydrogen atoms may optionally be replaced by halogen;
R¹ and R^{1′} which may be identical or different, denote a group selected from among hydrogen, C₁₋₆-alkyl, C₃₋₆-cycloalkyl, halogen-C₁₋₆-alkyl, halogen-C₃₋₆-cycloalkyl or C₁₋₆-alkylen-C₃₋₆-cycloalkyl, or
R¹ and R^{1′} together denote a C₂₋₅-alkylene bridge wherein one or more hydrogen atoms may optionally be replaced by halogen;
R², R^{2′}, R^{2″} and R^{2′″} which are identical or different, denote a group selected from among hydrogen, C₁₋₆-alkyl, halogen-C₁₋₆-alkylene, OH, HO—C₁₋₆-alkylene, —O—C₁₋₆-alkyl, C₆₋₁₀-aryl, C₆₋₁₀-aryl-C₁₋₄-alkylene, C₆₋₁₀-aryl-C₁₋₆-alkylene-O—, COOH, COOC₁₋₆-alkyl, O—C₁₋₆-alkylen-COOH, O—C₁₋₆-alkylene-COOC₁₋₆-alkyl, NHSO₂—C₁₋₆-alkyl, CN, NH₂, NH—C₁₋₆-alkyl, N(C₁₋₆-alkyl)₂, NO₂, S—C₁₋₆-alkyl, SO₂—C₁₋₆-alkyl, SO—C₁₋₆-alkyl, O(CO)C₁₋₆-alkyl, COC₁₋₆-alkyl, NHCOC₁₋₆-alkyl or halogen;
n denotes 0, 1 or 2; preferably 1;
   optionally in the form of the individual optical isomers, mixtures of the individual enantiomers or racemates, in the form of the free bases or the corresponding acid addition salts with pharmacologically acceptable acids, at least one additional active substance 2.

Preferably the present invention relates to medicament combinations which contain, in addition to one or more, preferably one, compound of formula 1 as an additional active substance 2 one or more compounds which are selected from the categories of the anticholinergics (2a), PDEIV-inhibitors (2b), steroids (2c), LTD4-antagonists (2d) and EGFR-inhibitors (2e).

Preferred medicament combinations are those which contain in addition to one or more, preferably one, compound of general formula 1, wherein
X denotes —O—, —CH₂—O—, —C(Me)₂-O— or —CH═CH—;
V denotes a double-bonded group selected from among CH₂, NH and O, preferably CH₂ and O, particularly preferably O;
R^{a} and R^{b} which are identical or different, denote a group selected from among hydrogen, C₁₋₄-alkyl and fluoro-C₁₋₄-alkyl,
   or
R^{a} and R^{b} together denote a group selected from —CH₂—CH₂—, —CH₂—CH₂—CH₂—CH₂— and —CH₂—CH₂—CH₂—CH₂—CH₂—, wherein one or more hydrogen atoms may optionally be replaced by fluorine or chlorine, preferably fluorine;
R¹ and R^{1′} which may be identical or different, denote a group selected from among hydrogen, C₁₋₆-alkyl, C₃₋₆-cycloalkyl, halogen-C₁₋₆-alkyl or C₁₋₆-alkylene-C₃₋₆-cycloalkyl,
   or
R¹ and R^{1′} together denote a group selected from —CH₂—CH₂—, —CH₂—CH₂—CH₂—CH₂— and —CH₂—CH₂—CH₂—CH₂—CH₂—, wherein one or more hydrogen atoms may optionally be replaced by fluorine or chlorine, preferably fluorine;
R², R^{2′}, R^{2″} and R^{2′″} which may be identical or different, denote a group selected from among hydrogen, C₁₋₄-alkyl, CF₃, CHF₂, CH₂F, OH, —O—C₁₋₄-alkyl, phenyl, phenylethyl, benzyl, phenyloxy, benzyloxy, COOH, COOC₁₋₄-alkyl, OCH₂COOH, OCH₂COOC₁₋₄-alkyl, NHSO₂—C₁₋₄-alkyl, fluorine, chlorine or bromine;
n denotes 0, 1 or 2; preferably 1;
   optionally in the form of the individual optical isomers, mixtures of the individual enantiomers or racemates, in the form of the free bases or the corresponding acid addition salts with pharmacologically acceptable acids, at least one additional active substance 2.

Preferred medicament combinations are those which contain in addition to one or more, preferably one, compound of general formula 1, wherein
X denotes —O—, —CH₂—O—, —C(Me)₂-O— or —CH═CH—;
V denotes a double-bonded group selected from among CH₂ and O, preferably O;
R^{a} and R^{b} which may be identical or different, denote a group selected from among hydrogen, methyl, ethyl and CF₃, preferably hydrogen, methyl or ethyl, or
R^{a} and R¹ together denote a group selected from —CH₂—CH₂— and —CH₂—CH₂—CH₂—CH₂, preferably —CH₂—CH₂—;
R¹ and R^{1′} which may be identical or different, denote a group selected from among hydrogen, methyl, ethyl, propyl, cyclopropyl or methylcyclopropyl,
   or
R¹ and R^{1′} together denote —CH₂—CH₂—CH₂—CH₂— or —CH₂—CH₂—CH₂—CH₂—CH₂—;
R², R^{2′}, R^{2″} and R^{2′″} which may be identical or different, denote a group selected from among hydrogen, methyl, ethyl, propyl, CF₃, CHF₂, CH₂F, OH, methyloxy, ethyloxy, propyloxy, COOH, COOCH₃, COOCH₂CH₃, OCH₂COOH, OCH₂COOCH₃, NHSO₂—CH₃, fluorine, chlorine or bromine;
n denotes 0, 1 or 2; preferably 1;
   optionally in the form of the individual optical isomers, mixtures of the individual enantiomers or racemates, in the form of the free bases or the corresponding acid addition salts with pharmacologically acceptable acids, at least one additional active substance 2.

Of particular importance according to the invention are medicament combinations which contain the compounds of formula 1 wherein R^{a} and R^{b} both denote methyl and wherein the groups X, R¹, R^{1′}, R², R^{2′}, R^{2″}, R^{2′″}, V and n may have the meanings given above. These preferred compounds may be represented by the following general formula 1.1 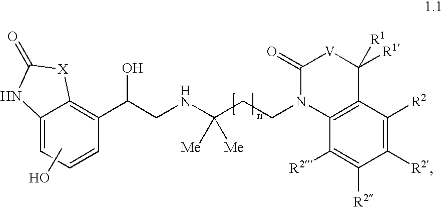
wherein the groups X, R¹, R^{1′}, R², R^{2′}, R^{2″}, R^{2′″}, V and n may have the meanings given above.

Of particular importance according to the invention are medicament combinations which contain the compounds of formula 1, wherein X corresponds to the group —CH₂—O—. These compounds may be represented by the formula 1′ 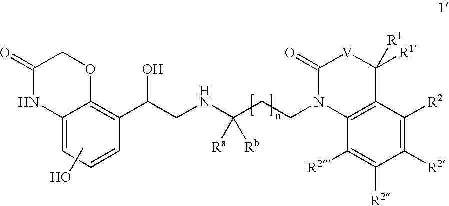
wherein the groups R^{a}, R^{b}, R¹, R^{1′}, R², R^{2′}, R^{2″}, R^{2′″} and V are as hereinbefore defined.

Preferred medicament combinations contain the compounds of formula 1′ wherein R^{a} and R^{b} may have the meanings given above and wherein
V denotes a double-bonded group selected from among CH₂ and O, preferably O;
R¹ and R^{1′} which may be identical or different, denote a group selected from among hydrogen, methyl, ethyl, propyl or cyclopropyl,
   or
R¹ and R^{1′} together denote —CH₂—CH₂—CH₂—CH₂— or —CH₂—CH₂—CH₂—CH₂—CH₂—;
R² and R^{2′″} denote hydrogen;
R^{2′} and R^{2″} which may be identical or different, denote a group selected from among hydrogen, methyl, CF₃, OH, methyloxy, benzyloxy, COOH, COOCH₃ or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Preferred medicament combinations according to the invention contain the compounds of formula 1′, wherein R^{a} and R^{b} may have the meanings given above and wherein
V denotes a double-bonded group selected from among CH₂ and O, preferably O;
R¹ and R^{1′} which may be identical or different denote hydrogen methyl, ethyl, propyl or cyclopropyl,
   or
R¹ and R^{1′} together denote —CH₂—CH₂—CH₂—CH₂—CH₂—;
R² and R^{2′″} denote hydrogen;
R^{2′} and R^{2″} which may be identical or different, denote a group selected from among hydrogen, methyl, CF₃, OH, methyloxy, benzyloxy or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Of equal importance according to the invention are medicament combinations containing the compounds of formula 1′, wherein R^{a} and R^{b} may have the meanings given above and wherein
V denotes the double-bonded group O;
R¹ and R^{1′} which may be identical or different, preferably identical, denote hydrogen, methyl, ethyl or propyl;
R², R^{2″} and R^{2″} denote hydrogen;
R^{2′} denotes hydrogen, OH, methyloxy or benzyloxy,
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of exceptional importance according to the invention are medicament combinations which contain compounds of formula 1′ wherein R^{a} and R^{b} may have the meanings given above and wherein
V denotes the double-bonded group O;
R¹ and R^{1′} in each case simultaneously represent hydrogen, methyl, ethyl or propyl;
R², R^{2″} and R^{2′″} denote hydrogen;
R^{2′} denotes hydrogen, OH or methyloxy,
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of particular importance according to the invention are medicament combinations which contain the compounds of formula 1′ wherein R^{a} and R^{b} both represent methyl. These compounds may be represented by the formula 1.1′ 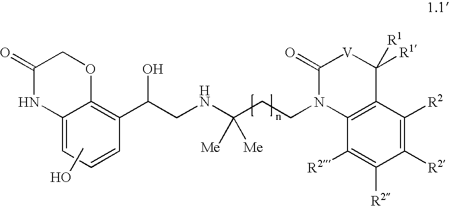
wherein the groups R¹, R^{1′}, R², R^{2′}, R^{2″}, R^{2′″} and V may have the meanings given above.

Preferred medicament combinations contain the compounds of formula 1, wherein X corresponds to the group —O—. These compounds may be represented by the formula 1″ 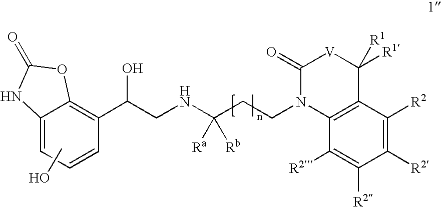
is wherein the groups R^{a}, R^{b}, R¹, R^{1′}, R², R^{2′}, R^{2″}, R^{2′″} and V are as hereinbefore defined.

Particularly preferred are medicament combinations which contain the compounds of formula 1″ wherein R^{a} and R^{b} may have the meanings given above and wherein
V denotes a double-bonded group selected from among CH₂ and O, preferably O;
R¹ and R^{1′} which may be identical or different, denote a group selected from among hydrogen, methyl, ethyl, propyl, cyclopropyl,
   or
R¹ and R^{1′} together denote —CH₂—CH₂—CH₂—CH₂— or —CH₂—CH₂—CH₂—CH₂—CH₂—;
R² and R^{2′″} denote hydrogen;
R^{2′} and R^{2″} which may be identical or different, denote a group selected from among hydrogen, methyl, CF₃, OH, methyloxy, benzyloxy, COOH, COOCH₃ or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also particularly preferred are medicament combinations which contain compounds of formula 1″, wherein R^{a} and R^{b} may have the meanings given above and wherein
V denotes a double-bonded group selected from among CH₂ and O, preferably O;
R¹ and R^{1′} which may be identical or different denote hydrogen methyl, ethyl or propyl,
   or
R¹ and R^{1′} together denote —CH₂—CH₂—CH₂—CH₂—CH₂—;
R² and R^{2′″} denote hydrogen;
R^{2′} and R^{2″} which may be identical or different, denote a group selected from among hydrogen, methyl, CF₃, OH, methyloxy, benzyloxy or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of equal importance according to the invention are medicament combinations which contain compounds of formula 1″ wherein R^{a} and R^{b} may have the meanings given above and wherein
V denotes the double-bonded group O;
R¹ and R^{1′} which may be identical or different, preferably identical, denote hydrogen, methyl or ethyl;
R², R^{2″} and R^{2″} denote hydrogen;
R^{2′} denotes hydrogen, OH, methyloxy or benzyloxy,
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of particular importance according to the invention are those medicament combinations which contain compounds of formula 1″ wherein R^{a} and R^{b} both represent methyl. These compounds may be represented by the formula 1.1″ 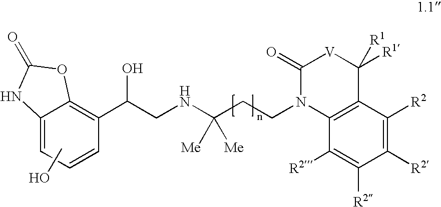
wherein the groups R¹, R^{1′}, R², R^{2′}, R^{2″}, R^{2′″} and V may have the meanings given above.

Also of particular importance according to the invention are medicament combinations which contain compounds of formula 1 wherein X corresponds to the group —CH═CH—. These compounds may be represented by the formula 1′″ 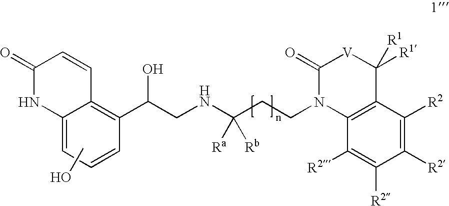
wherein the groups R^{a}, R^{b}, R¹, R^{1′}, R², R^{2′}, R^{2″}, R^{2′″} and V are as hereinbefore defined.

Preferred medicament combinations contain the compounds of formula 1′″, wherein R^{a} and R^{b} may have the meanings given above and wherein
V denotes a double-bonded group selected from among CH₂ and O, preferably O;
R¹ and R^{1′} which may be identical or different denote a group selected from among hydrogen, methyl, ethyl, propyl, cyclopropyl, preferably methyl or ethyl,
   or
R¹ and R^{1′} together denote —CH₂—CH₂—, —CH₂—CH₂—CH₂—CH₂— or —CH₂—CH₂—CH₂—CH₂—CH₂—;
R² and R^{2′″} denote hydrogen;
R^{2′} and R^{2″} which may be identical or different denote a group selected from among hydrogen, methyl, CF₃, OH, methyloxy, benzyloxy, COOH, COOCH₃ or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Particularly preferred are medicament combinations which contain compounds of formula 1′″ wherein R^{a} and R^{b} may have the meanings given above and wherein
V denotes a double-bonded group selected from among CH₂ and O, preferably O;
R¹ and R^{1′} which may be identical or different denote hydrogen, methyl, ethyl or propyl,
   or
R¹ and R^{1′} together denote —CH₂—CH₂—, —CH₂—CH₂—CH₂—CH₂— or —CH₂—CH₂—CH₂—CH₂—CH₂—;
R² and R^{2′″} denote hydrogen;
R^{2′} and R^{2″} which may be identical or different denote a group selected from among hydrogen, methyl, CF₃, OH, methyloxy, benzyloxy or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also particularly preferred are medicament combinations which contain compounds of formula 1′″ wherein R^{a} and R^{b} may have the meanings given above and wherein
V denotes the double-bonded group O;
R¹ and R^{1′} which may be identical or different, preferably identical, denote hydrogen, methyl, ethyl or propyl;
R², R^{2″} and R^{2″} denote hydrogen;
R^{2′} denotes hydrogen, OH, methyloxy or benzyloxy,
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of exceptional importance according to the invention are medicament combinations, the compounds of formula 1′″ contain, wherein R^{a} and R^{b} may have the meanings given above and wherein
V denotes the double-bonded group O;
R¹ and R^{1′} in each case simultaneously denote hydrogen, methyl, ethyl or propyl;
R², R^{2″} and R^{2′″} denote hydrogen;
R^{2′} denotes hydrogen, OH or methyloxy,
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of particular importance according to the invention are medicament combinations which contain compounds of formula 1′″ wherein R^{a} and R^{b} both represent methyl. These compounds may be represented by the formula 1.1′″ 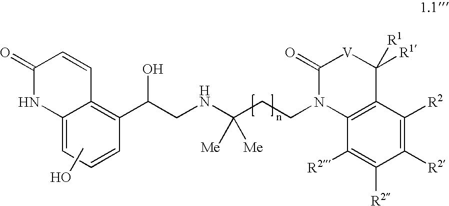
wherein the groups R¹, R^{1′}, R², R^{2′}, R^{2″}, R^{2′″} and V may have the meanings given above.

Also of particular importance according to the invention are medicament combinations which contain compounds of formula 1 wherein X denotes the group —CMe₂-O—. These compounds may be represented by the formula 1″″ 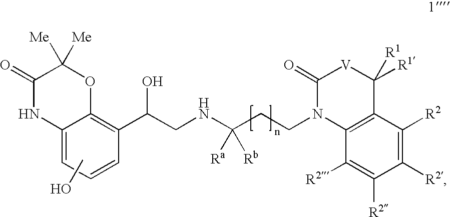
wherein the groups R^{a}, R^{b}, R¹, R^{1′}, R², R^{2′}, R^{2″}, R^{2′″} and V are as hereinbefore defined.

Preferred medicament combinations contain the compounds of formula 1″″, wherein R^{a} and R^{b} may have the meanings given above and wherein
V denotes a double-bonded group selected from among CH₂ and O, preferably O;
R¹ and R^{1′} which may be identical or different, denote a group selected from among hydrogen, methyl, ethyl, propyl, cyclopropyl,
   or
R¹ and R^{1′} together denote —CH₂—CH₂—CH₂—CH₂— or —CH₂—CH₂—CH₂—CH₂—CH₂—;
R² and R^{2′″} denote hydrogen;
R^{2′} and R^{2″} which may be identical or different, denote a group selected from among hydrogen, methyl, CF₃, OH, methyloxy, benzyloxy, COOH, COOCH₃ or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Particularly preferred are medicament combinations which contain compounds of formula 1″″ wherein R^{a} and R^{b} may have the meanings given above and wherein
V denotes a double-bonded group selected from among CH₂ and O, preferably O;
R¹ and R^{1′} which may be identical or different denote hydrogen, methyl, ethyl or propyl
   or
R¹ and R^{1′} together denote —CH₂—CH₂—CH₂—CH₂—CH₂—;
R² and R^{2′″} denote hydrogen;
R^{2′} and R^{2″} which may be identical or different denote a group selected from among hydrogen, methyl, CF₃, OH, methyloxy, benzyloxy or fluorine;
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also particularly preferred are medicament combinations which contain compounds of formula 1″″ wherein R^{a} and R^{b} may have the meanings given above and wherein
V denotes the double-bonded group O;
R¹ and R^{1′} which may be identical or different, preferably identical, denote hydrogen, methyl, ethyl or propyl;
R², R^{2″} and R^{2″} denote hydrogen;
R^{2′} denotes hydrogen, OH, methyloxy or benzyloxy,
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of exceptional importance according to the invention are medicament combinations which contain compounds of formula 1″″, wherein R^{a} and R^{b} may have the meanings given above and wherein
V denotes the double-bonded group O;
R¹ and R^{1′} in each case simultaneously denote hydrogen, methyl, ethyl or propyl;
R², R^{2″} and R^{2′″} denote hydrogen;
R^{2′} denotes hydrogen, OH or methyloxy,
n denotes 0, 1 or 2, preferably 1;
   optionally in the form of the individual optical isomers, mixtures of the individual enantiomers or racemates, in the form of the free bases or the corresponding acid addition salts with pharmacologically acceptable acids.

Also of particular importance according to the invention are those medicament combinations which contain compounds of formula 1″″ wherein R^{a} and R^{b} both represent methyl. These compounds may be represented by the formula 1.1″″ 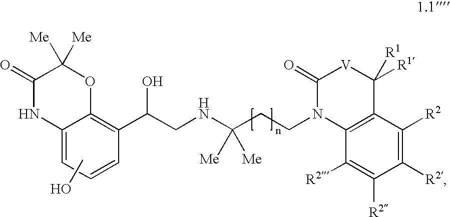
wherein the groups R¹, R^{1′}, R², R^{2′}, R^{2″}, R^{2′″} and V may have the meanings given above.

Also of exceptional importance according to the invention are medicament combinations which contain compounds of formula 1, wherein
R¹ and R^{1′} in each case simultaneously denotes hydrogen, methyl or ethyl, preferably methyl or ethyl, particularly preferably ethyl;
   and the groups X, R², R^{2′}, R^{2″}, R^{2′″}, V and n may have one of the meanings given above, optionally in the form of the individual optical isomers, mixtures of the individual enantiomers or racemates, in the form of the free bases or the corresponding acid addition salts with pharmacologically acceptable acids.

Also of exceptional importance according to the invention are medicament combinations which contain compounds of formula 1, wherein
n denotes 1
and the groups X, R¹, R^{1′}, R², R^{2′}, R^{2″}, R^{2′″} and V may have one of the meanings given above,
   optionally in the form of the individual optical isomers, mixtures of the individual enantiomers or racemates, in the form of the free bases or the corresponding acid addition salts with pharmacologically acceptable acids.

Particularly preferred medicament combinations are those which contain compounds of formula 1 selected from among:
1-{3-[2-hydroxy-2-(6-hydroxy-3-oxo-3,4-dihydro-2H-benzo[1,4]oxazin-8-yl)-ethylamino]-3-methyl-butyl}-6-methoxy-4,4-dimethyl-1,4-dihydro-benzo[d][1,3]oxazin-2-one (1.a),
6-hydroxy-1-{3-[2-hydroxy-2-(6-hydroxy-3-oxo-3,4-dihydro-2H-benzo[1,4]oxazin-8-yl)-ethylamino]-3-methyl-butyl}-4,4-dimethyl-1,4-dihydro-benzo[d][1,3]oxazin-2-one (1.b),
4,4-diethyl-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[d][1,3]oxazin-2-one (1.c),
6-hydroxy-8-{1-hydroxy-2-[3-(3-hydroxy-4,4-dimethyl-1-oxo-3,4-dihydro-1H-isoquinolin-2-yl)-1,1-dimethyl-propylamino]-ethyl}-4H-benzo[1,4]oxazin-3-one (1.d),
1-{-3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[1,3]oxazin-2-one (1.e),
4-ethyl-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[1,3]oxazin-2-one (1.f),
8-{2-[1,1-dimethyl-3-(2-oxo-3,4-dihydro-2H-quinolin-1-yl)-propylamino]-1-hydroxy-ethyl}-6-hydroxy-4H-benzo[1,4]oxazin-3-one (1.g),
4,4-diethyl-1-{3-[2-hydroxy-2-(8-hydroxy-2-oxo-1,2-dihydro-quinolin-5-yl)-ethylamino]-3-methyl-butyl}-1,4-dihydro-benzo[1,3]oxazin-2-one (1.h),
4,4-diethyl-1-{3-[2-hydroxy-2-(6-hydroxy-2,2-dimethyl-3-oxo-3,4-dihydro-2H-benzo[1,4]oxazin-8-yl)-ethylamino]-3-methyl-butyl}-1,4-dihydro-benzo[d][1,3]oxazin-2-one (1.j),
4,4-diethyl-1-{3-[2-hydroxy-2-(6-hydroxy-2,2-dimethyl-3-oxo-3,4-dihydro-2H-benzo[1,4]oxazin-8-yl)-ethylamino]-3-methyl-butyl}-1,4-dihydro-benzo[d][1,3]oxazin-2-one (1.j),
4,4-diethyl-1-{3-[2-hydroxy-2-(7-hydroxy-2-oxo-1,2-dihydro-quinolin-5-yl)-ethylamino]-3-methyl-butyl}-1,4-dihydro-benzo[d][1,3]oxazin-2-one (1.k),
1-{3-[2-hydroxy-2-(6-hydroxy-3-oxo-3,4-dihydro-2H-benzo[1,4]oxazin-8-yl)-ethylamino]-3-methyl-butyl}-4,4-dipropyl-1,4-dihydro-benzo[[1,3]oxazin-2-one (1.l),
1-{3-[2-hydroxy-2-(5-hydroxy-3-oxo-3,4-dihydro-2H-benzo[1,4]oxazin-8-yl)-ethylamino]-3-methyl-butyl}-4,4-dipropyl-1,4-dihydro-benzo[d][1,3]oxazin-2-one (1.m),
4,4-diethyl-7-fluoro-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[d][1,3]oxazin-2-one (1.n),
4,4-diethyl-1-{3-[2-hydroxy-2-(5-hydroxy-3-oxo-3,4-dihydro-2H-benzo[1,4]oxazin-8-yl)-ethylamino]-3-methyl-butyl}-8-methoxy-1,4-dihydro-benzo[d][1,3]oxazin-2-one (1.o),
1-(2-{1-[2-hydroxy-2-(5-hydroxy-3-oxo-3,4-dihydro-2H-benzo[1,4]oxazin-8-yl)-ethylamino]-cyclopropyl}-ethyl)-4,4-dimethyl-1,4-dihydro-benzo[d][1,3]oxazin-2-one (1.p),
1-(2-{1-[2-hydroxy-2-(6-hydroxy-3-oxo-3,4-dihydro-2H-benzo[1,4]oxazin-8-yl)-ethylamino]-cyclopropyl}-ethyl)-4,4-dimethyl-1,4-dihydro-benzo[d][1,3]oxazin-2-one (1.q),
1-{3-[2-hydroxy-2-(6-hydroxy-3-oxo-3,4-dihydro-2H-benzo[1,4]oxazin-8-yl)-ethylamino]-3-methyl-butyl}-spiro(cyclohexane-1,4′-2H-3′.1′-benzoxazin)-2′-one (1.r),
1-{3-[2-hydroxy-2-(5-hydroxy-3-oxo-3,4-dihydro-2H-benzo[1,4]oxazin-8-yl)-ethylamino]-3-methyl-butyl}-spiro(cyclohexane-1,4′-2H-3′,1′-benzoxazin)-2′-one (1.s),
4,4-dimethyl-1-{3-[2-hydroxy-2-(6-hydroxy-3-oxo-3,4-dihydro-2H-benzo[1,4]oxazin-8-yl)-ethylamino]-propyl}-1,4-dihydro-benzo[d][1,3]oxazin-2-one (1.t) and
4,4-dimethyl-1-{3-[2-hydroxy-2-(5-hydroxy-3-oxo-3,4-dihydro-2H-benzo[1,4]oxazin-8-yl)-ethylamino]-propyl}-1,4-dihydro-benzo[d][1,3]oxazin-2-one (1.u),
   optionally in the form of the individual optical isomers, mixtures of the individual enantiomers or racemates, in the form of the free bases or the corresponding acid addition salts with pharmacologically acceptable acids

The OH group may be configured in three different positions in the compounds of formula 1 defined hereinbefore. The isomers which may preferably be used in the combinations according to the invention may be represented by the following general formulae 1a and 1b, 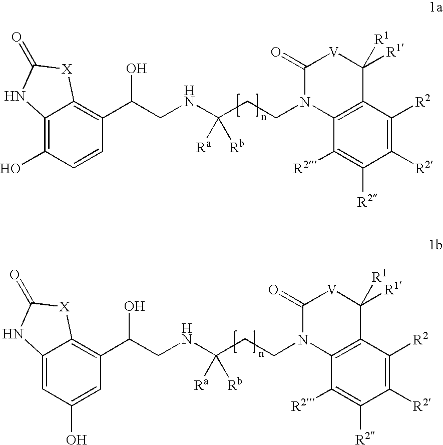
wherein the groups X, R^{a}, R^{b}, R¹, R^{1′}, R², R^{2′}, R^{2″}, R^{2′″}, V and n may have the meanings given above.

Particularly preferred medicament combinations also include, in particular, those which contain compounds of formula 1.1′-b, 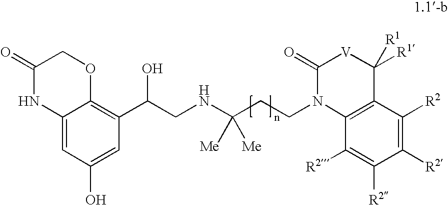
wherein the groups R¹, R^{1′}, R², R^{2′}, R^{2″}, R^{2′″} and V may have the meanings given above.

Particularly preferred medicament combinations also include, in particular, those which contain compounds of formula 1.1″-b, 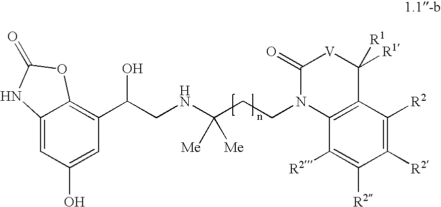
wherein the groups R¹, R^{1′}, R², R^{2′}, R^{2″}, R^{2′″} and V may have the meanings given above.

Particularly preferred medicament combinations also include, in particular, those which contain compounds of formula 1.1′″-a, 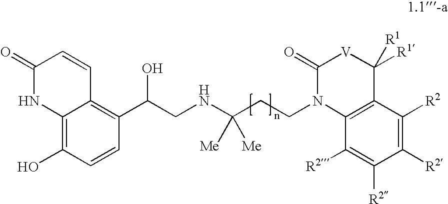
wherein the groups R¹, R^{1′}, R², R^{2′}, R^{2″}, R^{2′″} and V may have the meanings given above.

Particularly preferred medicament combinations also include, in particular, those which contain compounds of formula 1.1″″-b, 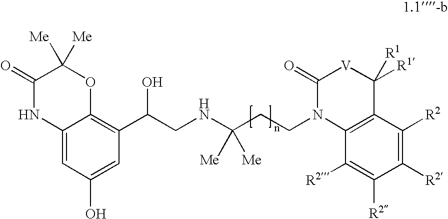
wherein the groups R¹, R^{1′}, R², R^{2′}, R^{2″}, R^{2′″} and V may have the meanings given above.

The compounds of formula 1 may optionally be used in the form of the individual optical isomers, mixtures of the individual enantiomers or racemates. Particularly preferably they are used in the form of the enantiomerically pure compounds, while the compounds of formula 1, wherein the asymmetric carbon centre “—CH(OH)—” in the benzyl position to the phenyl ring is in the R configuration. The particularly preferred R-enantiomers of the compounds of general formula 1 may be represented by the general formula R-1, 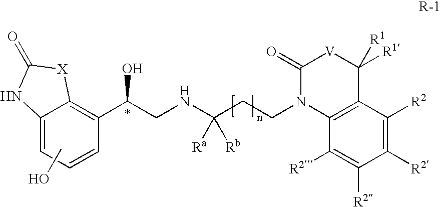
wherein the groups X, R^{a}, R^{b}, R¹, R^{1′}, R², R^{2′}, R^{2″}, R^{2′″}, V and n may have the meanings given above.

In another aspect the present invention relates to medicament combinations which contain the above-mentioned compounds of formula 1 in the form of the acid addition salts with pharmacologically acceptable acids as well as optionally in the form of the solvates and/or hydrates.

By acid addition salts with pharmacologically acceptable acids of the compounds 1 are meant, for example, salts selected from among the hydrochloride, hydrobromide, hydroiodide, hydrosulphate, hydrophosphate, hydromethanesulphonate, hydronitrate, hydromaleate, hydroacetate, hydrobenzoate, hydrocitrate, hydro fumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate. Of the above-mentioned acid addition salts the salts of hydrochloric acid, methanesulphonic acid, benzoic acid and acetic acid are particularly preferred according to the invention.

Preferred medicament combinations contain in addition to one or more, preferably one compound of formula 1, as an additional active substance, one or more, preferably one anticholinergic 2a, optionally in combination with pharmaceutically acceptable excipients.

In the medicament combinations according to the invention the anticholinergic 2a is preferably selected from among the tiotropium salts (2a.1), oxitropium salts (2a.2), flutropium salts (2a.3), ipratropium salts (2a.4), glycopyrronium salts (2a.5), trospium salts (2a.6) and the compounds of formulae 2a.7 to 2a.13.

In the above-mentioned salts 2a.1 to 2a.6 the cations tiotropium, oxitropium, flutropium, ipratropium, glycopyrronium and trospium represent the pharmacologically active constituents. Explicit reference to the above-mentioned cations is indicated by the terms 2a.1′ to 2a.6′. Any reference to the above-mentioned salts 2a.1 to 2a.6 naturally includes a reference to the corresponding cations tiotropium (2a.1′), oxitropium (2a.2′), flutropium (2a.3′), ipratropium (2a.4′), glycopyrronium (2a.5′), trospium (2a.6′).

By the salts 2a.1 to 2a.6 are meant, according to the invention, those compounds which contain in addition to the cations tiotropium (2a.1′), oxitropium (2a.2′), flutropium (2a.3′), ipratropium (2a.4′), glycopyrronium (2a.5′) and trospium (2a.6′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are the preferred counter-ions. Of all the salts, the chlorides, bromides, iodides and methanesulphonate are particularly preferred.

In the case of the trospium salts (2a.6) the chloride is particularly preferred. Of the other salts 2a.1 to 2a.5 the methanesulphonates and bromides are particularly important. Of particular importance are medicament combinations which contain tiotropium salts (2a.1), oxitropium salts (2a.2) or ipratropium salts (2a.4), while the respective bromides are particularly important according to the invention. Of particular importance is tiotropium bromide (2a.1). The above-mentioned salts may optionally be present in the medicament combinations according to the invention in the form of the solvates or hydrates thereof, preferably in the form of their hydrates. In the case of tiotropium bromide the medicament combinations according to the invention preferably contain this in the form of the crystalline tiotropium bromide monohydrate, which is known from WO 02/30928. If the tiotropium bromide is used in the medicament combinations according to the invention in anhydrous form, it is preferable to use the anhydrous crystalline tiotropium bromide which is known from WO 03/000265.

Examples of preferred medicament combinations of preferred compounds of formula 1 according to the invention containing the above-mentioned anticholinergics 2a.1 to 2a.6 are combinations containing the compounds 1.a and 2a.1; 1.a and 2a.2; 1.a and 2a.3; 1.a and 2a.4; 1.a and 2a.5; 1.a and 2a.6; 1.b and 2a.1; 1.b and 2a.2; 1.b and 2a.3; 1.b and 2a.4; 1.b and 2a.5; 1.b and 2a.6; 1.d and 2a.1; 1.d and 2a.2; 1.d and 2a.3; 1.d and 2a.4; 1.d and 2a.5; 1.d and 2a.6; 1.f and 2a.1; 1.f and 2a.2; 1.f and 2a.3; 1.f and 2a.4; 1.f and 2a.5; 1.f and 2a.6; 1.h and 2a.1; 1.h and 2a.2; 1.h and 2a.3; 1.h and 2a.4; 1.h and 2a.5; 1.h and 2a.6; 1.j and 2a.1; 1.j and 2a.2; 1.j and 2a.3; 1.j and 2a.4; 1.j and 2a.5; 1.j and 2a.6; 1.k and 2a.1; 1.k and 2a.2; 1.k and 2a.3; 1.k and 2a.4; 1.k and 2a.5; 1.k and 2a.6; 1.l and 2a1; 1.l and 2a.2; 1.l and 2a.3; 1.l and 2a.4; 1.l and 2a.5; 1.l and 2a.6; 1.m and 2a1; 1.m and 2a.2; 1.m and 2a.3; 1.m and 2a.4; 1.m and 2a.5; 1.m and 2a.6; 1.q and 2a.1; 1.q and 2a.2; 1.q and 2a.3; 1.q and 2a.4; 1.q and 2a.5 or 1.q and 2a.6 in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations other preferred ones according to the invention are those which contain as compound 2a one of the compounds 2a.1, 2a.2 or 2a.4, while the combinations which contain the compound 2a.1 are particularly important according to the invention.

The above-mentioned anticholinergics optionally contain chiral carbon centres. In this case the medicament combinations according to the invention may contain the anticholinergics in the form of the enantiomers, mixtures of enantiomers or racemates thereof, while preferably enantiomerically pure anticholinergics are used.

In another preferred embodiment of the present invention the anticholinergics 2a contained in the medicament combinations according to the invention are selected from the salts of formula 2a.7 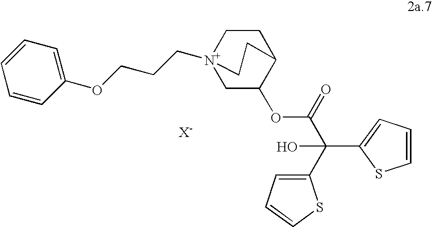
wherein
X⁻ denotes an anion with a single negative charge, preferably an anion selected from among fluorid, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof.

Preferred medicament combinations contain salts of formula 2a.7, wherein
X⁻ denotes an anion with a single negative charge, preferably an anion selected from among fluoride, chloride, bromide, methanesulphonate and p-toluenesulphonate, preferably bromide,
   optionally in the form of the racemates, enantiomers or hydrates thereof.

Preferred medicament combinations contain salts of formula 2a.7, wherein
X⁻ denotes an anion with a single negative charge, preferably an anion selected from among chloride, bromide and methanesulphonate, preferably bromide,
   optionally in the form of the racemates, enantiomers or hydrates thereof.

Particularly preferred medicament combinations contain the compound of formula 2a.7 in the form of the bromides.

Of particular importance are those medicament combinations which contain the enantiomers of formula 2a.7-en 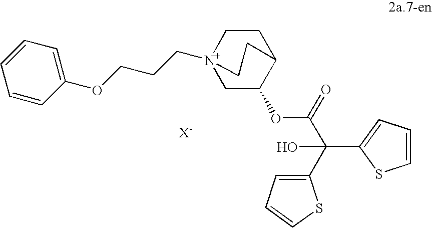
wherein X⁻ may have the meanings given above.

Examples of medicament combinations of preferred compounds of formula 1 according to the invention containing the above-mentioned anticholinergics 2a.7 are combinations containing the compounds 1.a and 2a.7; 1.a and 2a.7-en; 1.b and 2a.7; 1.b and 2a.7-en; 1.d and 2a.7; 1.d and 2a.7-en; 1.f and 2a.7; 1.f and 2a.7-en; 1.h and 2a.7; 1.h and 2a.7-en; 1.j, and 2a.7; 1.j and 2a.7-en; 1.k and 2a.7; 1.k and 2a.7-en; 1.l and 2a.7; 1.l and 2a.7-en; 1.m and 2a.7; 1.m and 2a.7-en; 1.q and 2a.7; 1.q and 2a.7-en, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations according to the invention those which contain the compound 2a.7-en as compound 2a are also preferred.

In another preferred embodiment of the present invention the anticholinergics 2a contained in the medicament combinations according to the invention are selected from the salts of formula 2a.8 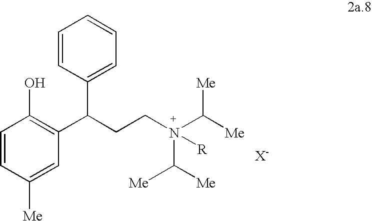
wherein R denotes either methyl (2a.8.1) or ethyl (2a.8.2) and wherein X⁻ may have the meanings given above. In an alternative embodiment the compound of formula 2a.8 is present in the form of the free base 2a.8-base

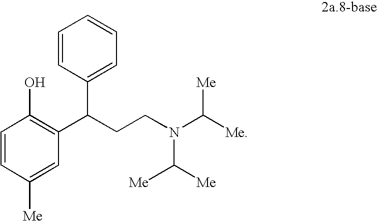

The medicament combinations according to the invention may contain the anticholinergic of formula 2a.8 (or 2a.8-base) in the form of the enantiomers, mixtures of enantiomers or racemates thereof. Preferably the anticholinergics of formula 2a.8 (or 2a.8-base) are present in the form of their R-enantiomers.

Examples of medicament combinations of preferred compounds of formula 1 according to the invention containing the above-mentioned anticholinergics 2a.8 are combinations containing the compounds 1.a and 2a.8.1; 1.a and 2a.8.2; 1.b and 2a.8.1; 1.b and 2a.8.2; 1.d and 2a.8.1; 1.d and 2a.8.2; 1.f and 2a.8.1; 1.f and 2a.8.2; 1.h and 2a.8.1; 1.h and 2a.8.2; 1.j and 2a.8.1; 1.j and 2a.8.2; 1.k and 2a.8.1; 1.k and 2a.8.2; 1.l and 2a.8.1; 1.l and 2a.8.2; 1.m and 2a.8.1; 1.m and 2a.8.2; 1.q and 2a.8.1; 1.q and 2a.8.2, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention.

In another preferred embodiment of the present invention the anticholinergics 2a contained in the medicament combinations according to the invention are selected from the compounds of formula 2a.9 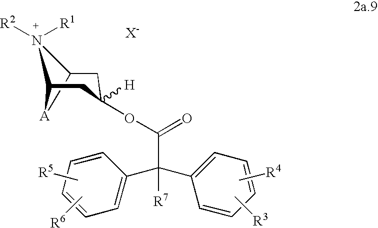
wherein
A denotes a double-bonded group selected from among the groups 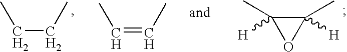
X⁻ denotes one of the above-mentioned anions with a single negative charge, preferably chloride, bromide or methanesulphonate,
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine.

The compounds of formula 2a.9 are known in the art (WO 02/32899).

Preferred compounds of formula 2a.9 within the scope of the medicament combinations is according to the invention are those wherein
X⁻ denotes bromide;
R¹ and R² which may be identical or different, denote methyl or ethyl, preferably methyl;
R³, R⁴, R⁵ and R⁶, which may be identical or different, denote hydrogen, methyl, methyloxy, chlorine or fluorine;
R⁷ denote hydrogen, methyl or fluorine.

Of particular importance are medicament combinations which contain compounds of formula 2a.9 wherein
A denotes a double-bonded group selected from among

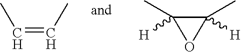

Of particular importance are those medicament combinations which contain, in addition to a compound of formula 1, one of the following compounds of formula 2a.9:
tropenol 2,2-diphenylpropionate methobromide (2a.9.1),
scopine 2,2-diphenylpropionate methobromide (2a.9.2),
scopine 2-fluoro-2,2-diphenylacetate methobromide (2a.9.3),
tropenol 2-fluoro-2,2-diphenylacetate methobromide (2a.9.4);

The compounds of formula 2a.9 may optionally be present in the form of their enantiomers, mixtures of their enantiomers or racemates, as well as optionally in the form of the hydrates and/or solvates thereof.

Examples of medicament combinations of preferred compounds of formula 1 according to the invention containing the above-mentioned anticholinergics 2a.9 are combinations containing the compounds 1.a and 2a.9.1; 1.a and 2a.9.2; 1.a and 2a.9.3; 1.a and 2a.9.4; 1.b and 2a.9.1; 1.b and 2a.9.2; 1.b and 2a.9.3; 1.b and 2a.9.4; 1.d and 2a.9.1; 1.d and 2a.9.2; 1.d and 2a.9.3; 1.d and 2a.9.4; 1.f and 2a.9.1; 1.f and 2a.9.2; 1.f and 2a.9.3; 1.f and 2a.9.4; 1.h and 2a.9.1; 1.h and 2a.9.2; 1.h and 2a.9.3; 1.h and 2a.9.4; 1.j and 2a.9.1; 1.j and 2a.9.2; 1.j and 2a.9.3; 1.j and 2a.9.4; 1.k and 2a.9.1; 1.k and 2a.9.2; 1.k and 2a.9.3; 1.k and 2a.9.4; 1.l and 2a.9.1; 1.l and 2a.9.2; 1.l and 2a.9.3; 1.l and 2a.9.4; 1.m and 2a.9.1; 1.m and 2a.9.2; 1.m and 2a.9.3; 1.m and 2a.9.4; 1.q and 2a.9.1; 1.q and 2a.9.2; 1.q and 2a.9.3; 1.q and 2a.9.4, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations other preferred ones according to the invention are those which contain as compound 2a.9 one of the compounds 2a.9.1 or 2a.9.2, while the combinations containing the compound 2a.9.2 are particularly important according to the invention.

In another preferred embodiment of the present invention the anticholinergics 2a contained in the medicament combinations according to the invention are selected from the compounds of formula 2a.10 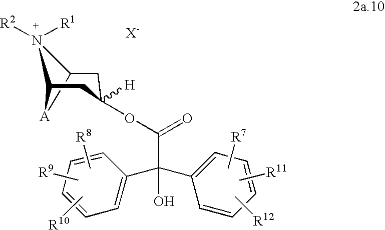
wherein
A, X, R¹ and R² may have the meanings given above and wherein
R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 2a.10 are known in the art (WO 02/32898).

Within the scope of the medicament combinations according to the invention preferred compounds of formula 2a.10 are those wherein
A denotes a double-bonded group selected from 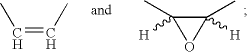
X⁻ denotes bromide;
R¹ and R² which may be identical or different, denote methyl or ethyl, preferably methyl;
R⁷, R⁸, R⁹, R¹⁰, R¹¹ and R¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those medicament combinations which contain in addition to a compound of formula 1 one of the following compounds of formula 2a.10:
tropenol 3,3′,4,4′-tetrafluorobenzilate methobromide (2a.10.1),
scopine 3,3′,4,4′-tetrafluorobenzilate methobromide (2a.10.2),
tropenol 4,4′-difluorobenzilate methobromide (2a.10.3),
scopine 4,4′-difluorobenzilate methobromide (2a.10.4),
tropenol 3,3′-difluorobenzilate methobromide (2a.10.5),
scopine 3,3′-difluorobenzilate methobromide (2a.10.6).

The compounds of formula 2a.10 may optionally be present in the form of the enantiomers thereof, mixtures of the enantiomers or racemates thereof, and optionally in the form of the hydrates and/or solvates thereof.

Examples of medicament combinations of preferred compounds of formula 1 according to the invention containing the above-mentioned anticholinergics 2a.10 are combinations containing the compounds 1.a and 2a.10.1; 1.a and 2a.10.2; 1.a and 2a.10.3; 1.a and 2a.10.4; 1.a and 2a.10.5; 1.a and 2a.10.6; 1.b and 2a.10.1; 1.b and 2a.10.2; 1.b and 2a.10.3; 1.b and 2a.10.4; 1.b and 2a.10.5; 1.b and 2a.10.6; 1.d and 2a.10.1; 1.d and 2a.10.2; 1.d and 2a.10.3; 1.d and 2a.10.4; 1.d and 2a.10.5; 1.d and 2a.10.6; 1.f and 2a.10.1; 1.f and 2a.10.2; 1.f and 2a.10.3; 1.f and 2a.10.4; 1.f and 2a.10.5; 1.f and 2a.10.6; 1.h and 2a.10.1; 1.h and 2a.10.2; 1.h and 2a.10.3; 1.h and 2a.10.4; 1.h and 2a.10.5; 1.h and 2a.10.6; 1.j and 2a.10.1; 1.j and 2a.10.2; 1.j and 2a.10.3; 1.j and 2a.10.4; 1.j and 2a.10.5; 1.j and 2a.10.6; 1.k and 2a.10.1; 1.k and 2a.10.2; 1.k and 2a.10.3; 1.k and 2a.10.4; 1.k and 2a.10.5; 1.k and 2a.10.6; 1.l and 2a.10.1; 1.l and 2a.10.2; 1.l and 2a.10.3; 1.l and 2a.10.4; 1.l and 2a.10.5; 1.l and 2a.10.6; 1.m and 2a.10.1; 1.m and 2a.10.2; 1.m and 2a.10.3; 1.m and 2a.10.4; 1.m and 2a.10.5; 1.m and 2a.10.6; 1.q and 2a.10.1; 1.q and 2a.10.2; 1.q and 2a.10.3; 1.q and 2a.10.4; 1.q and 2a.10.5; 1.q and 2a.10.6, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations according to the invention those which contain as compound 2a.10 one of the compounds 2a.10.1, 2a.10.2, 2a.10.3 or 2a.10.4 are also preferred, while the combinations which contain the compounds 2a.10.1 or 2a.10.2 are particularly important according to the invention.

In another preferred embodiment of the present invention the anticholinergics 2a contained in the medicament combinations according to the invention are selected from the compounds of formula 2a.11 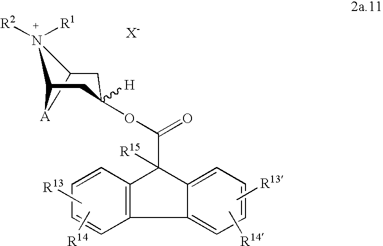
wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen,
   or
   R^{1′} and R^{2′} together denote a —C₃-C₅-alkylene bridge;
R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen.

The compounds of formula 2a.11 are known in the art (WO 03/064419).

Within the scope of the medicament combinations according to the invention preferred compounds of formula 2a.11 are those wherein
A denotes a double-bonded group selected from 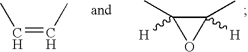
X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different, denote methyl or ethyl, preferably methyl;
R¹³, R¹⁴, R^{13′} and R^{14′} which may be identical or different, denote hydrogen, —CF₃, —CHF₂ or fluorine, preferably hydrogen or fluorine.

Within the scope of the medicament combinations according to the invention particularly preferred compounds of formula 2a.11 are those wherein
A denotes a double-bonded group selected from 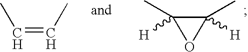
   X⁻ denotes bromide;
R¹⁵ denotes hydroxy or methyl, preferably methyl;
R^{1′} and R^{2′} which may be identical or different, denote methyl or ethyl, preferably methyl;
R¹³, R¹⁴, R^{13′} and R^{14′} which may be identical or different, denote hydrogen or fluorine.

Of particular importance are those medicament combinations which contain in addition to a compound of formula 1 one of the following compounds of formula 2a.11:
tropenol 9-hydroxy-fluorene-9-carboxylate methobromide (2a.11.1);
tropenol 9-fluoro-fluorene-9-carboxylate methobromide (2a.11.2);
scopine 9-hydroxy-fluorene-9-carboxylate methobromide (2a.11.3);
scopine 9-fluoro-fluorene-9-carboxylate methobromide (2a.11.4);
tropenol 9-methyl-fluorene-9-carboxylate methobromide (2a.11.5);
scopine 9-methyl-fluorene-9-carboxylate methobromide (2a.11.6);

The compounds of formula 2a.11 may optionally be present in the form of the enantiomers thereof, mixtures of the enantiomers or racemates thereof, and optionally in the form of the hydrates and/or solvates thereof.

Examples of medicament combinations of preferred compounds of formula 1 according to the invention containing the above-mentioned anticholinergics 2a.11 are combinations containing the compounds 1.a and 2a.11.1; 1.a and 2a.11.2; 1.a and 2a.11.3; 1.a and 2a.11.4; 1.a and 2a.11.5; 1.a and 2a.11.6; 1.b and 2a.11.1; 1.b and 2a.11.2; 1.b and 2a.11.3; 1.b and 2a.11.4; 1.b and 2a.11.5; 1.b and 2a.11.6; 1.d and 2a.11.1; 1.d and 2a.11.2; 1.d and 2a.11.3; 1.d and 2a.11.4; 1.d and 2a.11.5; 1.d and 2a.11.6; 1.f and 2a.11.1; 1.f and 2a.11.2; 1.f and 2a.11.3; 1.f and 2a.11.4; 1.f and 2a.11.5; 1.f and 2a.11.6; 1.h and 2a.11.1; 1.h and 2a.11.2; 1.h and 2a.11.3; 1.h and 2a.11.4; 1.h and 2a.11.5; 1.h and 2a.11.6; 1.j and 2a.11.1; 1.j and 2a.11.2; 1.j and 2a.11.3; 1.j and 2a.11.4; 1.j and 2a.11.5; 1.j and 2a.11.6; 1.k and 2a.11.1; 1.k and 2a.11.2; 1.k and 2a.11.3; 1.k and 2a.11.4; 1.k and 2a.11.5; 1.k and 2a.11.6; 1.l and 2a.11.1; 1.l and 2a.11.2; 1.l and 2a.11.3; 1.l and 2a.11.4; 1.l and 2a.11.5; 1.l and 2a.11.6; 1.m and 2a.11.1; 1.m and 2a.11.2; 1.m and 2a.11.3; 1.m and 2a.11.4; 1.m and 2a.11.5; 1.m and 2a.11.6; 1.q and 2a.11.1; 1.q and 2a.11.2; 1.q and 2a.11.3; 1.q and 2a.11.4; 1.q and 2a.11.5; 1.q and 2a.11.6, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations those which contain as compound 2a.11 one of the compounds 2a.11.2, 2a.11.4, 2a.11.5 or 2a.11.6 according to the invention are also preferred, while the combinations which contain the compounds 2a.11.5 or 2a.11.6 are particularly important according to the invention.

In another preferred embodiment of the present invention the anticholinergics 2a contained in the medicament combinations according to the invention are selected from the compounds of formula 2a.12 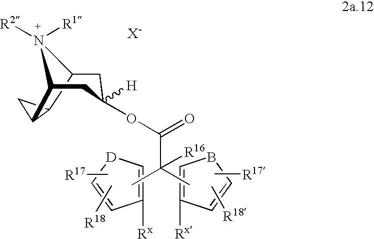
wherein X⁻ may have the meanings given above and wherein
D and B which may be identical or different, preferably identical, denote O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen,
   or
   R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{x} and R^{x′} which may be identical or different, denote hydrogen, —C₁-C₄-alkyl, —C₁-C₄-alkyloxy, hydroxy, —CF₃, —CHF₂, CN, NO₂ or halogen,
   or
   R^{x} and R^{x′} together denote a single bond or one of the double-bonded groups denotes O, S, NH, CH₂, CH₂—CH₂, N(C₁-C₄-alkyl), CH(C₁-C₄-alkyl) and —C(C₁-C₄-alkyl)₂.

The compounds of formula 2a.12 are known in the art (WO 03/064418).

Within the scope of the medicament combinations according to the invention preferred compounds of formula 2a.12 are those wherein
X⁻ denotes chloride, bromide or methanesulphonate, preferably bromide;
D and B which may be identical or different, preferably identical, denote O, S, NH or CH═CH;
R¹⁶ denotes hydrogen, hydroxy, —C₁-C₄-alkyl, —C₁-C₄-alkyloxy, —CF₃, —CHF₂, fluorine, chlorine or bromine;
R^{1″} and R^{2″} which may be identical or different, denote C₁-C₄-alkyl, which may optionally be substituted by hydroxy, fluorine, chlorine or bromine,
   or
   R^{1″} and R^{2″} together denote a —C₃-C₄-alkylene bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂, fluorine, chlorine or bromine;
R^{x} and R^{x′} which may be identical or different, denote hydrogen, C₁-C₄-alkyl, C₁-C₄-alkyloxy, hydroxy, —CF₃, —CHF₂, CN, NO₂, fluorine, chlorine or bromine,
   or
   R^{x} and R^{x′} together denote a single bond or a double-bonded group selected from O, S, NH— and CH₂.

Within the scope of the medicament combinations according to the invention particularly preferred compounds of formula 2a.12 are those wherein
X⁻ denotes chloride, bromide, or methanesulphonate, preferably bromide;
D and B which may be identical or different, preferably identical, denotes S or CH═CH;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different, denote hydrogen, —CF₃ or fluorine, preferably hydrogen, or
   R^{x} and R^{x′} together denote a single bond or —O—.

Within the scope of the medicament combinations according to the invention particularly preferred compounds of formula 2a.12 also include those wherein
X⁻ denotes bromide;
D and B denote —CH═CH—;
R¹⁶ denotes hydrogen, hydroxy or methyl;
R^{1″} and R^{2″} denote methyl;
R¹⁷, R¹⁸, R^{17′} and R^{18′}, which may be identical or different, denote hydrogen or fluorine, preferably hydrogen;
R^{x} and R^{x′} which may be identical or different, denote hydrogen or fluorine, preferably hydrogen, or
   R^{x} and R^{x′} together denote a single bond or the group —O—.

Of particular importance are those medicament combinations which contain in addition to a compound of formula 1 one of the following compounds of formula 2a.12:
cyclopropyltropine benzilate methobromide (2a.12.1);
cyclopropyltropine 2,2-diphenylpropionate methobromide (2a.12,2);
cyclopropyltropine 9-hydroxy-xanthene-9-carboxylate methobromide (2a.12.3);
cyclopropyltropine 9-methyl-fluorene-9-carboxylate methobromide (2a.12.4);
cyclopropyltropine 9-methyl-xanthene-9-carboxylate methobromide (2a.12.5);
cyclopropyltropine 9-hydroxy-fluorene-9-carboxylate methobromide (2a.12.6);
methyl cyclopropyltropine 4,4′-difluorobenzilate methobromide (2a.12.7).

The compounds of formula 2a.12 may optionally be present in the form of the enantiomers thereof, mixtures of the enantiomers or racemates thereof, and optionally in the form of the hydrates and/or solvates thereof.

Examples of medicament combinations of preferred compounds of formula 1 according to the invention containing the above-mentioned anticholinergics 2a.12 are combinations containing the compounds 1.a and 2a.12.1; 1.a and 2a.12,2; 1.a and 2a.12.3; 1.a and 2a.12.4; 1.a and 2a.12.5; 1.a and 2a.12.6; 1.a and 2a.12.7; 1.b and 2a.12.1; 1.b and 2a.12.2; 1.b and 2a.12.3; 1.b and 2a.12.4; 1.b and 2a.12.5; 1.b and 2a.12.6; 1.b and 2a.12.7; 1.d and 2a.12.1; 1.d and 2a.12.2; 1.d and 2a.12.3; 1.d and 2a.12.4; 1.d and 2a.12.5; 1.d and 2a.12.6; 1.d and 2a.12.7; 1.f and 2a.12.1; 1.f and 2a.12.2; 1.f and 2a.12.3; 1.f and 2a.12.4; 1.f and 2a.12.5; 1.f and 2a.12.6; 1.f and 2a.12.7; 1.h and 2a.12.1; 1.h and 2a.12.2; 1.h and 2a.12.3; 1.h and 2a.12.4; 1.h and 2a.12.5; 1.h and 2a.12.6; 1.h and 2a.12.7; 1.j and 2a.12.1; 1.j and 2a.12.2; 1.j and 2a.12.3; 1.j and 2a.12.4; 1.j and 2a.12.5; 1.j and 2a.12.6; 1.j and 2a.12.7; 1.k and 2a.12.1; 1.k and 2a.12.2; 1.k and 2a.12.3; 1.k and 2a.12.4; 1.k and 2a.12.5; 1.k and 2a.12.6; 1.k and 2a.12.7; 1.l and 2a.12.1; 1.l and 2a.12.2; 1.l and 2a.12.3; 1.l and 2a.12.4; 1.l and 2a.12.5; 1.l and 2a.12.6; 1.l and 2a.12.7; 1.m and 2a.12.1; 1.m and 2a.12.2; 1.m and 2a.12.3; 1.m and 2a.12.4; 1.m and 2a.12.5; 1.m and 2a.12.6; 1.m and 2a.12.7; 1.q and 2a.12.1; 1.q and 2a.12.2; 1.q and 2a.12.3; 1.q and 2a.12.4; 1.q and 2a.12.5; 1.q and 2a.12,6; 1.q and 2a.12.7, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations those which contain as compound 2a.11 one of the compounds 2a.12.1, 2a.12.2, 2a.12.5 or 2a.12.7 are also preferred according to the invention, while the combinations which contain the compounds 2a.12.1 or 2a.12.2 are particularly important according to the invention.

In another preferred embodiment of the present invention the anticholinergics 2a contained in the medicament combinations according to the invention are selected from the compounds of formula 2a.13 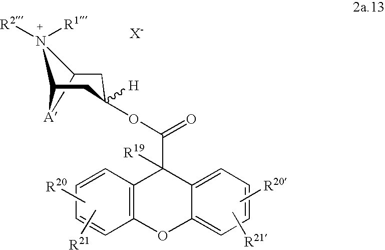
wherein X⁻ may have the meanings given above and wherein
A′ denotes a double-bonded group selected from 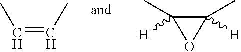
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen,
   or
   R^{1′″} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen.

The compounds of formula 2a.13 are known in the art (WO 03/064417).

Within the scope of the medicament combinations according to the invention preferred compounds of formula 2a.13 are those wherein
A′ denotes a double-bonded group selected from 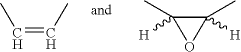
X⁻ denotes chloride, bromide or methanesulphnat, preferably bromide;
R¹⁹ denotes hydroxy or methyl;
R^{1′″} and R^{2′″} which may be identical or different, denote methyl or ethyl, preferably methyl;
R²⁰, R²¹, R^{20′} and R^{21′} which may be identical or different, denote hydrogen, —CF₃, —CHF₂ or fluorine, preferably hydrogen or fluorine.

Within the scope of the medicament combinations according to the invention particularly preferred compounds of formula 2a.13 are those wherein
A′ denotes a double-bonded group selected from 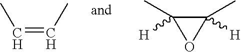
   X⁻ denotes bromide;
R¹⁹ denotes hydroxy or methyl, preferably methyl;
R^{1′″} and R^{2′″} which may be identical or different, denote methyl or ethyl, preferably methyl;
R²⁰, R²¹, R^{20′} and R^{21′} which may be identical or different, denote hydrogen or fluorine.

Of particular importance are those medicament combinations which contain in addition to a compound of formula 1 one of the following compounds of formula 2a.13:
tropenol 9-hydroxy-xanthene-9-carboxylate methobromide (2a.13.1);
scopine 9-hydroxy-xanthene-9-carboxylate methobromide (2a.13.2);
tropenol 9-methyl-xanthene-9-carboxylate methobromide (2a.13.3);
scopine 9-methyl-xanthene-9-carboxylate methobromide (2a.13.4);
tropenol 9-ethyl-xanthene-9-carboxylate methobromide (2a.13.5);
tropenol 9-difluoromethyl-xanthene-9-carboxylate methobromide (2a.13.6);
scopine 9-hydroxymethyl-xanthene-9-carboxylate methobromide (2a.13.7).

The compounds of formula 2a.13 may optionally be present in the form of the enantiomers thereof, mixtures of the enantiomers or racemates thereof, and optionally in the form of the hydrates and/or solvates thereof.

Examples of medicament combinations of preferred compounds of formula 1 according to the invention containing the above-mentioned anticholinergics 2a.13 are combinations containing the compounds 1.a and 2a.13.1; 1.a and 2a.13.2; 1.a and 2a.13.3; 1.a and 2a.13.4; 1.a and 2a.13.5; 1.a and 2a.13.6; 1.a and 2a.13.7; 1.b and 2a.13.1; 1.b and 2a.13.2; 1.b and 2a.13.3; 1.b and 2a.13.4; 1.b and 2a.13.5; 1.b and 2a.13.6; 1.b and 2a.13.7; 1.d and 2a.13.1; 1.d and 2a.13.2; 1.d and 2a.13.3; 1.d and 2a.13.4; 1.d and 2a.13.5; 1.d and 2a.13.6; 1.d and 2a.13.7; 1.f and 2a.13.1; 1.f and 2a.13.2; 1.f and 2a.13.3; 1.f and 2a.13.4; 1.f and 2a.13.5; 1.f and 2a.13.6; 1.f and 2a.13.7; 1.h and 2a.13.1; 1.h and 2a.13.2; 1.h and 2a.13.3; 1.h and 2a.13.4; 1.h and 2a.13.5; 1.h and 2a.13.6; 1.h and 2a.13.7; 1.j and 2a.13.1; 1.j and 2a.13.2; 1.j and 2a.13.3; 1.j and 2a.13.4; 1.j and 2a.13.5; 1.j and 2a.13.6; 1.j and 2a.13.7; 1.k and 2a.13.1; 1.k and 2a.13.2; 1.k and 2a.13.3; 1.k and 2a.13.4; 1.k and 2a.13.5; 1.k and 2a.13.6; 1.k and 2a.13.7; 1.l and 2a.13.1; 1.l and 2a.13.2; 1.l and 2a.13.3; 1.l and 2a.13.4; 1.l and 2a.13.5; 1.l and 2a.13.6; 1.l and 2a.13.7; 1.m and 2a.13.1; 1.m and 2a.13.2; 1.m and 2a.13.3; 1.m and 2a.13.4; 1.m and 2a.13.5; 1.m and 2a.13.6; 1.m and 2a.13.7; 1.q and 2a.13.1; 1.q and 2a.13.2; 1.q and 2a.13.3; 1.q and 2a.13.4; 1.q and 2a.13.5; 1.q and 2a.13.6; 1.q and 2a.13.7, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations those which contain as compound 2a.11 one of the compounds 2a.13.2, 2a.13.3, 2a.13.4 or 2a.13.5 are also preferred according to the invention, while the combinations which contain the compounds 2a.13.3 or 2a.13.4 are particularly important according to the invention.

Within the scope of the present invention any reference to anticholinergics 1′ should be understood as a reference to the pharmacologically active cations of the respective salts. These cations are tiotropium (2a.1′), oxitropium (2a.2′), flutropium (2a.3′), ipratropium (2a.4′), glycopyrronium (2a.5′), trospium (2a.6′) and the following cations

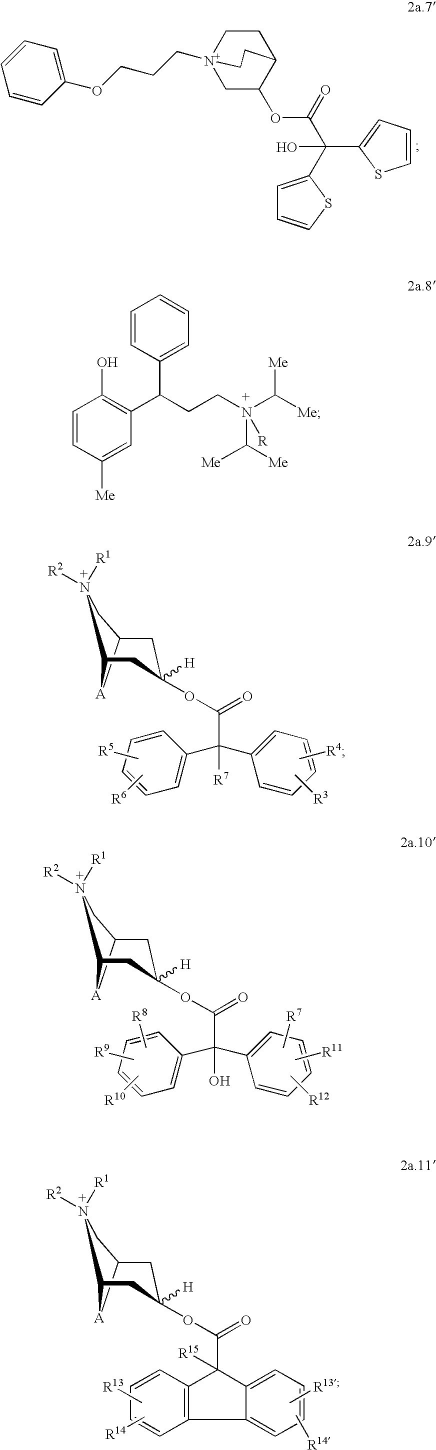

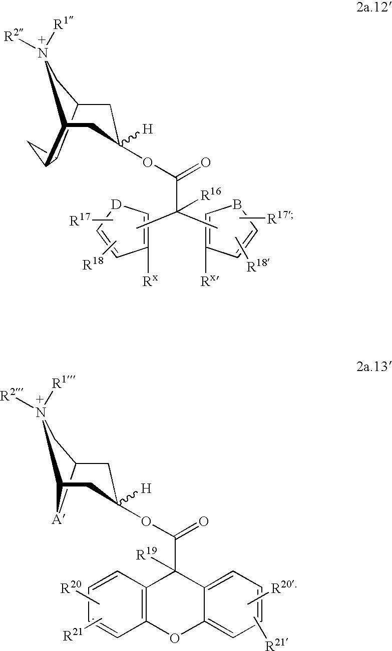

Other medicament combinations which are preferred according to the invention contain in addition to one or more, preferably one compound of formula 1, as an additional active substance, one or more, preferably one PDE IV-inhibitor 2b, optionally in combination with pharmaceutically acceptable excipients.

In medicament combinations of this kind the PDE IV inhibitor 2b is preferably selected from among enprofyllin, theophyllin, roflumilast, ariflo (cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)p-[(4aR*, 10bS*)-9-ethoxy-1,2,3,4,4a,10b-hexahydro-8-methoxy-2-methylbenzo[s][1.6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-5-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, C1-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among enprofyllin (2b.1), roflumilast (2b.2), ariflo (cilomilast) (2b.3), AWD-12-281 (GW-842470) (2b.4, N-(3,5-dichloro-1-oxo-pyridin-4-yl)-4-difluoromethoxy-3-cyclopropylmethoxybenzamide (2b.5), T-440 (2b.6), T-2585 (2b.7), arofyllin (2b.8), cis[4-cyano-4-(3-cyclopentyloxy-4-methoxyphenyl)cyclohexane-1-carboxylic acid](2b.9), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol](2b.11), PD-168787 (2b.12), atizoram (2b.13), V-11294A (2b.14), C1-1018 (2b.15), CDC-801 (2b.16, D-22888 (2b.17), YM-58997 (2b.18), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among roflumilast (2b.2), ariflo (cilomilast) (2b.3), AWD-12-281 (GW-842470) (2b.4), arofyllin (2b.8), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol](2b.11), atizoram (2b.13), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), while roflumilast (2b.2), Z-15370 (2b.19) and AWD-12-281 (2b.4) are of particular importance, optionally in the form of the racemates, enantiomers or diastereomers thereof and optionally in the form of the pharmacologically acceptable acid addition salts, solvates and/or hydrates thereof.

By acid addition salts with pharmacologically acceptable acids which the compounds 2b may be capable of forming are meant, for example, salts selected from among hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

Examples of preferred medicament combinations of preferred compounds of formula 1 with the above-mentioned PDE IV-inhibitors 2b include combinations containing the compounds 1.a and 2b.1; 1.a and 2b.2; 1.a and 2b.3; 1.a and 2b.4; 1.a and 2b.5; 1.a and 2b.6; 1.a and 2b.7; 1.a and 2b.8; 1.a and 2b.9; 1.a and 2b.10; 1.a and 2b.11; 1.a and 2b.12; 1.a and 2b.13; 1.a and 2b.14; 1.a and 2b.15; 1.a and 2b.16; 1.a and 2b.17; 1.a and 2b.18; 1.a and 2b.19; 1.a and 2b.20; 1.a and 2b.21; 1.b and 2b.1; 1.b and 2b.2; 1.b and 2b.3; 1.b and 2b.4; 1.b and 2b.5; 1.b and 2b.6; 1.b and 2b.7; 1.b and 2b.8; 1.b and 2b.9; 1.b and 2b.10; 1.b and 2b.11; 1.b and 2b.12; 1.b and 2b.13; 1.b and 2b.14; 1.b and 2b.15; 1.b and 2b.16; 1.b and 2b.17; 1.b and 2b.18; 1.b and 2b.19; 1.b and 2b.20; 1.b and 2b.21; 1.d and 2b.1; 1.d and 2b.2; 1.d and 2b.3; 1.d and 2b.4; 1.d and 2b.5; 1.d and 2b.6; 1.d and 2b.7; 1.d and 2b.8; 1.d and 2b.9; 1.d and 2b.10; 1.d and 2b.11; 1.d and 2b.12; 1.d and 2b.13; 1.d and 2b.14; 1.d and 2b.15; 1.d and 2b.16; 1.d and 2b.17; 1.d and 2b.18; 1.d and 2b.19; 1.d and 2b.20; 1.d and 2b.21; 1.f and 2b.1; 1.f and 2b.2; 1.f and 2b.3; 1.f and 2b.4; 1.f and 2b.5; 1.f and 2b.6; 1.f and 2b.7; 1.f and 2b.8; 1.f and 2b.9; 1.f and 2b.10; 1.f and 2b.11; 1.f and 2b.12; 1.f and 2b.13; 1.f and 2b.14; 1.f and 2b.15; 1.f and 2b.16; 1.f and 2b.17; 1.f and 2b.18; 1.f and 2b.19; 1.f and 2b.20; 1.f and 2b.21; 1.h and 2b.1; 1.h and 2b.2; 1.h and 2b.3; 1.h and 2b.4; 1.h and 2b.5; 1.h and 2b.6; 1.h and 2b.7; 1.h and 2b.8; 1.h and 2b.9; 1.h and 2b.10; 1.h and 2b.11; 1.h and 2b.12; 1.h and 2b.13; 1.h and 2b.14; 1.h and 2b.15; 1.h and 2b.16; 1.h and 2b.17; 1.h and 2b.18; 1.h and 2b.19; 1.h and 2b.20; 1.h and 2b.21; 1.j and 2b.1; 1.j and 2b.2; 1.j and 2b.3; 1.j and 2b.4; 1.j and 2b.5; 1.j and 2b.6; 1.j and 2b.7; 1.j and 2b.8; 1.j and 2b.9; 1.j and 2b.10; 1.j and 2b.11; 1.j and 2b.12; 1.j and 2b.13; 1.j and 2b.14; 1.j and 2b.15; 1.j and 2b.16; 1.j and 2b.17; 1.j and 2b.18; 1.j and 2b.19; 1.j and 2b.20; 1.j and 2b.21; 1.k and 2b.1; 1.k and 2b.2; 1.k and 2b.3; 1.k and 2b.4; 1.k and 2b.5; 1.k and 2b.6; 1.k and 2b.7; 1.k and 2b.8; 1.k and 2b.9; 1.k and 2b.10; 1.k and 2b.11; 1.k and 2b.12; 1.k and 2b.13; 1.k and 2b.14; 1.k and 2b.15; 1.k and 2b.16; 1.k and 2b.17; 1.k and 2b.18; 1.k and 2b.19; 1.k and 2b.20; 1.k and 2b.21; 1.l and 2b.1; 1.l and 2b.2; 1.l and 2b.3; 1.l and 2b.4; 1.l and 2b.5; 1.l and 2b.6; 1.l and 2b.7; 1.l and 2b.8; 1.l and 2b.9; 1.l and 2b.10; 1.l and 2b.11; 1.l and 2b.12; 1.l and 2b.13; 1.l and 2b.14; 1.l and 2b.15; 1.l and 2b.16; 1.l and 2b.17; 1.l and 2b.18; 1.l and 2b.19; 1.l and 2b.20; 1.l and 2b.21; 1.m and 2b.1; 1.m and 2b.2; 1.m and 2b.3; 1.m and 2b.4; 1.m and 2b.5; 1.m and 2b.6; 1.m and 2b.7; 1.m and 2b.8; 1.m and 2b.9; 1.m and 2b.10; 1.m and 2b.11; 1.m and 2b.12; 1.m and 2b.13; 1.m and 2b.14; 1.m and 2b.15; 1.m and 2b.16; 1.m and 2b.17; 1.m and 2b.18; 1.m and 2b.19; 1.m and 2b.20; 1.m and 2b.21; 1.q and 2b.1; 1.q and 2b.2; 1.q and 2b.3; 1.q and 2b.4; 1.q and 2b.5; 1.q and 2b.6; 1.q and 2b.7; 1.q and 2b.8; 1.q and 2b.9; 1.q and 2b.10; 1.q and 2b.11; 1.q and 2b.12; 1.q and 2b.13; 1.q and 2b.14; 1.q and 2b.15; 1.q and 2b.16; 1.q and 2b.17; 1.q and 2b.18; 1.q and 2b.19; 1.q and 2b.20 or 1.q and 2b.21, in each case optionally in the form of the racemates, enantiomers or diastereomers thereof and optionally in the form of the pharmacologically acceptable acid addition salts, solvates and/or hydrates thereof, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations those which contain as compound 2b one of the compounds 2b.2, 2b.3, 2b.4, 2b.8, 2b.10, 2b.11, 2b.13, 2b.19, 2b.20 or 2b.21 are also preferred according to the invention, while the combinations which contain one of the compounds 2b.2, 2b.4 or 2b.19 are particularly important according to the invention.

Other preferred medicament combinations according to the invention contain in addition to one or more, preferably one compound of formula 1, as an additional active substance, one or more, preferably one steroid 2c, optionally in combination with pharmaceutically acceptable excipients.

In medicament combinations of this kind the steroid 2c is preferably selected from among prednisolone (2c.1), prednisone (2c.2), butixocortpropionate (2c.3), RPR-106541 (2c.4), flunisolide (2c.5), beclomethasone (2c.6), triamcinolone (2c.7, budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl)6α,9α-difluoro-11β-hydroxy-16α-methyl-3-oxo-17α-propionyloxy-androsta-1,4-diene-17β-carbothionate (2c.16) and etiprednol-dichloroacetate (BNP-166, 2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among flunisolide (2c.5), beclomethasone (2c.6), triamcinolone (2c..7, budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among budesonide (2c.8), fluticasone (2c.9, mometasone (2c.10), ciclesonide (2c.11), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15) and etiprednol-dichloroacetate (2c.17), optionally in the form of the racemates, enantiomers or diastereomers thereof and optionally in the form of the salts and derivatives thereof, the solvates and/or hydrates thereof.

Any reference to steroids 2c includes a reference to any salts or derivatives, hydrates or solvates thereof which may exist. Examples of possible salts and derivatives of the steroids 2c may be: alkali metal salts, such as for example sodium or potassium salts, sulphobenzoates, phosphates, isonicotinates, acetates, propionates, dihydrogen phosphates, palmitates, pivalates or also furoates.

Examples of preferred medicament combinations of preferred compounds of formula 1 with the above-mentioned steroids 2c are combinations containing the compounds 1.a and 2c.1; 1.a and 2c.2; 1.a and 2c.3; 1.a and 2c.4; 1.a and 2c.5; 1.a and 2c.6; 1.a and 2c.7; 1.a and 2c.8; 1.a and 2c.9; 1.a and 2c.10; 1.a and 2c.11; 1.a and 2c.12; 1.a and 2c.13; 1.a and 2c.14; 1.a and 2c.15; 1.a and 2c.16; 1.a and 2c.17; 1.b and 2c.1; 1.b and 2c.2; 1.b and 2c.3; 1.b and 2c.4; 1.b and 2c.5; 1.b and 2c.6; 1.b and 2c.7; 1.b and 2c.8; 1.b and 2c.9; 1.b and 2c.10; 1.b and 2c.11; 1.b and 2c.12; 1.b and 2c.13; 1.b and 2c.14; 1.b and 2c.15; 1.b and 2c.16; 1.b and 2c.17; 1.d and 2c.1; 1.d and 2c.2; 1.d and 2c.3; 1.d and 2c.4; 1.d and 2c.5; 1.d and 2c.6; 1.d and 2c.7; 1.d and 2c.8; 1.d and 2c.9; 1.d and 2c.10; 1.d and 2c.11; 1.d and 2c.12; 1.d and 2c.13; 1.d and 2c.14; 1.d and 2c.15; 1.d and 2c.16; 1.d and 2c.17; 1.f and 2c.1; 1.f and 2.2; 1.f and 2c.3; 1.f and 2c.4; 1.f and 2c.5; 1.f and 2c.6; 1.f and 2c.7; 1.f and 2c.8; 1.f and 2c.9; 1.f and 2c.10; 1.f and 2c.11; 1.f and 2c.12; 1.f and 2c.13; 1.f and 2c.14; 1.f and 2c.15; 1.f and 2c.16; 1.f and 2c.17; 1.h and 2c.1; 1.h and 2c.2; 1.h and 2c.3; 1.h and 2c.4; 1.h and 2c.5; 1.h and 2c.6; 1.h and 2c.7; 1.h and 2c.8; 1.h and 2c.9; 1.h and 2c.10; 1.h and 2c.11; 1.h and 2c.12; 1.h and 2c.13; 1.h and 2c.14; 1.h and 2c.15; 1.h and 2c.16; 1.h and 2c.17; 1.j and 2c.1; 1.j and 2c.2; 1.j and 2c.3; 1.j and 2c.4; 1.j and 2c.5; 1.j and 2c.6; 1.j and 2c.7; 1.j and 2c.8; 1.j and 2c.9; 1.j and 2c.10; 1.j and 2c.11; 1.j and 2c.12; 1.j and 2c.13; 1.j and 2c.14; 1.j and 2c.15; 1.j and 2c.16; 1.j and 2c.17; 1.k and 2c.1; 1.k and 2c.2; 1.k and 2c.3; 1.k and 2c.4; 1.k and 2c.5; 1.k and 2c.6; 1.k and 2c.7; 1.k and 2c.8; 1.k and 2c.9; 1.k and 2c.10; 1.k and 2c.11; 1.k and 2c.12; 1.k and 2c.13; 1.k and 2c.14; 1.k and 2c.15; 1.k and 2c.16; 1.k and 2c.17; 1.l and 2c.1; 1.l and 2c.2; 1.l and 2c.3; 1.l and 2c.4; 1.l and 2c.5; 1.l and 2c.6; 1.l and 2c.7; 1.l and 2c.8; 1.l and 2c.9; 1.l and 2c.10; 1.l and 2c.11; 1.l and 2c.12; 1.l and 2c.13; 1.l and 2c.14; 1.l and 2c.15; 1.l and 2c.16; 1.l and 2c.17; 1.m and 2c.1; 1.m and 2c.2; 1.m and 2c.3; 1.m and 2c.4; 1.m and 2c.5; 1.m and 2c.6; 1.m and 2c.7; 1.m and 2c.8; 1.m and 2c.9; 1.m and 2.10; 1.m and 2c.11; 1.m and 2c.12; 1.m and 2c.13; 1.m and 2c.14; 1.m and 2c.15; 1.m and 2c.16; 1.m and 2c.17; 1.q and 2c.1; 1.q and 2c.2; 1.q and 2c.3; 1.q and 2c.4; 1.q and 2c.5; 1.q and 2c.6; 1.q and 2c.7; 1.q and 2c.8; 1.q and 2c.9; 1.q and 2c.10; 1.q and 2c.11; 1.q and 2c.12; 1.q and 2c.13; 1.q and 2c.14; 1.q and 2c.15; 1.q and 2c.16 or 1.q and 2c.17 in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a 1.h, 1.j, or 1.k, while the combinations which contain one of the compounds 1.h or 1.k are particularly important according to the invention. Of the above-mentioned combinations those which contain as compound 2c one of the compounds 2c.5, 2c.6, 2c.7, 2c.8, 2c.9, 2c.10, 2c.11, 2c.12, 2c.13, 2c.14, 2c.15, 2c.16 or 2c.17 are also preferred according to the invention, while the combinations which contain one of the compounds 2c.8, 2c.9, 2c.10, 2c.11, 2c.15 or 2c.17 are particularly important according to the invention.

Other preferred medicament combinations according to the invention contain in addition to one or more, preferably one compound of formula 1 as an additional active substance one or more, preferably one LTD4-antagonist 2d, optionally in combination with pharmaceutically acceptable excipients.

In medicament combinations of this kind the LTD4-antagonist 2d is preferably selected from among montelukast (2d.1), 1-(((R)-(3-(2-(6,7-difluoro-2-quinolinyl)ethenyl)phenyl)-3-(2-(2-hydroxy-2-propyl)phenyl)thio)methylcyclopropaneacetic acid (2d.2), 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid (2d.3), pranlukast (2d.4), zafirlukast (2d.5), [2-[[2-(4-tert-butyl-2-thiazolyl)-5-benzofuranyl]oxymethyl]phenyl]acetic acid (2d.6), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In preferred medicament combinations the LTD4-antagonist 2d is selected from among montelukast (2d.1), pranlukast (2d.4), zafirlukast (2d.5), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In particularly preferred medicament combinations the LTD4-antagonist 2d is selected from among montelukast (2d.1), pranlukast (2d.4), zafirlukast (2d.5), MCC-847 (ZD-3523) (2d.7), MN-001 (2d.8) and MEN-91507 (LM-1507) (2d.9), while montelukast (2d.1), pranlukast (2d.4 and zafirlukast (2d.5) are particularly preferred, optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

By acid addition salts with pharmacologically acceptable acids which the compounds 2d may be capable of forming are meant, for example, salts selected from among hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluolsulphonate, preferably hydrochloride, hydrobromide, hydrosulphate, hydrophosphate, hydrofumarate and hydromethanesulphonate.

By salts or derivatives which the compounds 2d may be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or also furoates.

Examples of preferred medicament combinations of preferred compounds of formula 1 according to the invention with the above-mentioned LTD4-antagonists 2d are combinations containing the compounds 1.a and 2d.1; 1.a and 2d.2; 1.a and 2d.3; 1.a and 2d.4; 1.a and 2d.5; 1.a and 2d.6; 1.a and 2d.7; 1.a and 2d.8; 1.a and 2d.9; 1.a and 2d.10; 1.a and 2d.11; 1.a and 2d.12; 1.b and 2d.1; 1.b and 2d.2; 1.b and 2d.3; 1.b and 2d.4; 1.b and 2d.5; 1.b and 2d.6; 1.b and 2d.7; 1.b and 2d.8; 1.b and 2d.9; 1.b and 2d.10; 1.b and 2d.11; 1.b and 2d.12; 1.d and 2d.1; 1.d and 2d.2; 1.d and 2d.3; 1.d and 2d.4; 1.d and 2d.5; 1.d and 2d.6; 1.d and 2d.7; 1.d and 2d.8; 1.d and 2d.9; 1.d and 2d.10; 1.d and 2d.11; 1.d and 2d.12; 1.f and 2d.1; 1.f and 2d.2; 1.f and 2d.3; 1.f and 2d.4; 1.f and 2d.5; 1.f and 2d.6; 1.f and 2d.7; 1.f and 2d.8; 1.f and 2d.9; 1.f and 2d.10; 1.f and 2d.11; 1.f and 2d.12; 1.h and 2d.1; 1.h and 2d.2; 1.h and 2d.3; 1.h and 2d.4; 1.h and 2d.5; 1.h and 2d.6; 1.h and 2d.7; 1.h and 2d.8; 1.h and 2d.9; 1.h and 2d.10; 1.h and 2d.11; 1.h and 2d.12; 1.j and 2d.1; 1.j and 2d.2; 1.j and 2d.3; 1.j and 2d.4; 1.j and 2d5; 1.j and 2d.6; 1.j and 2d.7; 1.j and 2d.8; 1.j and 2d.9; 1.j and 2d.10; 1.j and 2d.11; 1.j and 2d.12; 1.k and 2d.1; 1.k and 2d.2; 1.k and 2d.3; 1.k and 2d.4; 1.k and 2d.5; 1.k and 2d.6; 1.k and 2d.7; 1.k and 2d.8; 1.k and 2d.9; 1.k and 2d.10; 1.k and 2d.11; 1.k and 2d.12; 1.l and 2d.1; 1.l and 2d.2; 1.l and 2d.3; 1.l and 2d.4; 1.l and 2d.5; 1.l and 2d.6; 1.l and 2d.7; 1.l and 2d.8; 1.l and 2d.9; 1.l and 2d.10; 1.l and 2d.11; 1.l and 2d.12; 1.m and 2d.1; 1.m and 2d.2; 1.m and 2d.3; 1.m and 2d.4; 1.m and 2d.5; 1.m and 2d.6; 1.m and 2d.7; 1.m and 2d.8; 1.m and 2d.9; 1.m and 2d.10; 1.m and 2d.11; 1.m and 2d.12; 1.q and 2d.1; 1.q and 2d.2; 1.q and 2d.3; 1.q and 2d.4; 1.q and 2d.5; 1.q and 2d.6; 1.q and 2d.7; 1.q and 2d.8; 1.q and 2d.9; 1.q and 2d.10; 1.q and 2d.11 or 1.q and 2d.12, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations those which contain as compound 2d one of the compounds 2d.1, 2d.4, 2d.5, 2d.7, 2d.8, 2d.9, 2d.10, 2d.11 or 2d.12 are also preferred according to the invention, while the combinations which contain one of the compounds 2d.1, 2d.4, 2d.5, 2d.7, 2d.8 or 2d.9 are particularly important according to the invention, while the combinations which contain one of the compounds 2d.1, 2d.4 or 2d.5 are of exceptional importance.

Other preferred medicament combinations according to the invention contain, in addition to one or more, preferably one compound of formula 1, as an additional active substance, one or more, preferably one EGFR-inhibitor 2e, optionally in combination with pharmaceutically acceptable excipients.

In medicament combinations of this kind the EGFR-inhibitor 2e is selected for example from among 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidin, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3 chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethansulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, cetuximab, trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

In medicament combinations of this kind the EGFR-inhibitor 2e is preferably selected from among 4-[(3-chloro-4-fluorophenyl)amino]-6-{[4-(morpholin-4-yl)-1-oxo-2-buten-1-yl]-amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro4-fluorophenyl)amino]-6-({4-[N-(2-methoxy-ethyl)-N-methyl-amino]-1-oxo2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidin, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4 [(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethansulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1 acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7-(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and cetuximab, optionally in the form of the racemates, enantiomers or diastereomers thereof, optionally in the form of the pharmacologically acceptable acid addition salts thereof, the solvates and/or hydrates thereof.

It is particularly preferred within the scope of the medicament combinations according to the invention to use EGFR-inhibitors 2a selected from among 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidin, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4 [(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethansulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydro furan-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

Particularly preferred medicament combinations according to the invention contain as EGFR-inhibitors 2e those compounds which are selected from among:
4-[(3-chloro-4-fluorophenyl)amino]-6-{[4-(morpholin-4-yl)-1-oxo-2-buten-1-yl]-amino}-7-cyclopropylmethoxy-quinazoline (2e.1),
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline (2e.2),
4-[(3-chloro-4-fluoro-phenyl)amino]-6-[2-((S)-6-methyl-2-oxo-morpholin-4-yl)-ethoxy]-7-methoxy-quinazoline (2e.3),
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline (2e.4),
4-[(3-ethynyl-phenyl)amino]-6,7-bis-(2-methoxy-ethoxy)-quinazoline (2e.5),
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline (2e.6),
4-[(3-ethynyl-phenyl)amino]-6-{[4-(5,5-dimethyl-2-oxo-morpholin-4-yl)-1-oxo-2-buten-1-yl]amino}-quinazoline (2e.7),
4-[(3-chloro-4-fluoro-phenyl)amino]-6-(trans-4-methanesulphonylamino-cyclohexan-1-yloxy)-7-methoxy-quinazoline (2e.8),
4-[(3-chloro-4-fluoro-phenyl)amino]-6-(tetrahydropyran-3-yloxy)-7-methoxy-quinazoline (2e.9),
4-[(3-chloro-4-fluoro-phenyl)amino]-6-{1-[(morpholin-4-yl)carbonyl]-piperidin-4-yl-oxy}-7-methoxy-quinazoline (2e.10),
4-[(3-chloro-4-fluoro-phenyl)amino]-6-{1-[2-(2-oxopyrrolidin-1-yl)ethyl]-piperidin-4-yloxy}-7-methoxy-quinazoline (2e.11),
4-[(3-ethynyl-phenyl)amino]-6-(1-acetyl-piperidin-4-yloxy)-7-methoxy-quinazoline (2e.12),
4-[(3-ethynyl-phenyl)amino]-6-(1-methyl-piperidin-4-yloxy)-7-methoxy-quinazoline (2e.13),
4-[(3-ethynyl-phenyl)amino]-6-(1-methanesulphonyl-piperidin-4-yloxy)-7-methoxy-quinazoline (2e.14),
4-[(3-ethynyl-phenyl)amino]-6-{1-[(morpholin-4-yl)carbonyl]-piperidin-4-yloxy}-7-methoxy-quinazoline (2e.15),
4-[(3-chloro-4-fluoro-phenyl)amino]-6-{1-[(2-methoxyethyl)carbonyl]-piperidin-4-yloxy}-7-methoxy-quinazoline (2e.16),
4-[(3-chloro-4-fluoro-phenyl)amino]-6-[cis-4-(N-methanesulphonyl-N-methyl-amino)-cyclohexan-1-yloxy]-7-methoxy-quinazoline (2e.17),
4-[(3-chloro-4-fluoro-phenyl)amino]-6-[cis-4-(N-acetyl-N-methyl-amino)-cyclohexan-1-yloxy]-7-methoxy-quinazoline (2e.18),
4-[(3-chloro-4-fluoro-phenyl)amino]-6-(trans-4-methylamino-cyclohexan-1-yloxy)-7-methoxy-quinazoline (2e.19),
4-[(3-chloro-4-fluoro-phenyl)amino]-6-[trans-4-(N-methanesulphonyl-N-methyl-amino)-cyclohexan-1-yloxy]-7-methoxy-quinazoline (2e.20),
4-[(3-chloro-4-fluoro-phenyl)amino]-6-(trans-4-dimethylamino-cyclohexan-1-yloxy)-7-methoxy-quinazoline (2e.21),
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline (2e.22),
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline (2e.23),
4-[(3-chloro-4-fluoro-phenyl)amino]-6-(1-methanesulphonyl-piperidin-4-yloxy)-7-methoxy-quinazoline (2e.24 and
4-[(3-chloro-4-fluoro-phenyl)amino]-6-(1-cyano-piperidin-4-yloxy)-7-methoxy-quinazoline (2e25),
   optionally in the form of the racemates, enantiomers or diastereomers thereof, optionally in the form of the pharmacologically acceptable acid addition salts thereof, the solvates and/or hydrates thereof.

By acid addition salts with pharmacologically acceptable acids which the compounds 2e may be capable of forming are meant, for example, salts selected from among hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

Examples of preferred medicament combinations of preferred compounds of formula 1 according to the invention with the above-mentioned EGFR-inhibitors 2e are combinations containing the compounds 1.a and 2e.1; 1.a and 2e.2; 1.a and 2e.3; 1.a and 2e.4; 1.a and 2e.5; 1.a and 2e.6; 1.a and 2e.7; 1.a and 2e.8; 1.a and 2e.9; 1.a and 2e.10; 1.a and 2e.11; 1.a and 2e.12; 1.a and 2e.13; 1.a and 2e.14; 1.a and 2e.15; 1.a and 2e.16; 1.a and 2e.17; 1.a and 2e.18; 1.a and 2e.19; 1.a and 2e.20; 1.a and 2e.21; 1.a and 2e.22; 1.a and 2e.23; 1.a and 2e.24; 1.a and 2e.25; 1.b and 2e.1; 1.b and 2e.2; 1.b and 2e.3; 1.b and 2e.4; 1.b and 2e.5; 1.b and 2e.6; 1.b and 2e.7; 1.b and 2e.8; 1.b and 2e.9; 1.b and 2e.10; 1.b and 2e.11; 1.b and 2e.12; 1.b and 2e.13; 1.b and 2e.14; 1.b and 2e.15; 1.b and 2e.16; 1.b and 2e.17; 1.b and 2e.18; 1.b and 2e1.9; 1.b and 2e.20; 1.b and 2e.21; 1.b and 2e.22; 1.b and 2e.23; 1.b and 2e.24; 1.b and 2e.25; 1.d and 2e.1; 1.d and 2e.2; 1.d and 2e.3; 1.d and 2e.4; 1.d and 2e.5; 1.d and 2e.6; 1.d and 2e.7; 1.d and 2e.8; 1.d and 2e.9; 1.d and 2e.10; 1.d and 2e.11; 1.d and 2e.12; 1.d and 2e.13; 1.d and 2e.14; 1.d and 2e.15; 1.d and 2e.16; 1.d and 2e.17; 1.d and 2e.18; 1.d and 2e.19; 1.d and 2e.20; 1.d and 2e.21; 1.d and 2e.22; 1.d and 2e.23; 1.d and 2e.24; 1.d and 2e.25; 1.f and 2e.1; 1.f and 2e.2; 1.f and 2e.3; 1.f and 2e.4; 1.f and 2e.5; 1.f and 2e.6; 1.f and 2e.7; 1.f and 2e.8; 1.f and 2e.9; 1.f and 2e.10; 1.f and 2e.11; 1.f and 2e.12; 1.f and 2e.13; 1.f and 2e.14; 1.f and 2e.15; 1.f and 2e.16; 1.f and 2e.17; 1.f and 2e.18; 1.f and 2e.19; 1.f and 2e.20; 1.f and 2e.21; 1.f and 2e.22; 1.f and 2e.23; 1.f and 2e.24; 1.f and 2e.25; 1.h and 2e.1; 1.h and 2e.2; 1.h and 2e.3; 1.h and 2e.4; 1.h and 2e.5; 1.h and 2e.6; 1.h and 2e.7; 1.h and 2e.8; 1.h and 2e.9; 1.h and 2e.10; 1.h and 2e.11; 1.h and 2e.12; 1.h and 2e.13; 1.h and 2e.14; 1.h and 2e.15; 1.h and 2e.16; 1.h and 2e.17; 1.h and 2e.18; 1.h and 2e.19; 1.h and 2e.20; 1.h and 2e.21; 1.h and 2e.22; 1.h and 2e.23; 1.h and 2e.24; 1.h and 2e.25; 1.j and 2e.1; 1.j and 2e.2; 1.j and 2e.3; 1.j and 2e.4; 1.j and 2e.5; 1.j and 2e.6; 1.j and 2e.7; 1.j and 2e.8; 1.j and 2e.9; l.j and 2e.10; 1.j and 2e.11; 1.j and 2e.12; 1.j and 2e.13; 1.j and 2e.14; 1.j and 2e.15; 1.j and 2e.16; 1.j and 2e.17; 1.j and 2e.18; 1.j and 2e.19; 1.j and 2e.20; 1.j and 2e.21; 1.j and 2e.22; 1.j and 2e.23; 1.j and 2e.24; 1.j and 2e.25; 1.k and 2e.1; 1.k and 2e.2; 1.k and 2e.3; 1.k and 2e.4; 1.k and 2e.5; 1.k and 2e.6; 1.k and 2e.7; 1.k and 2e.8; 1.k and 2e.9; 1.k and 2e.10; 1.k and 2e.11; 1.k and 2e.12; 1.k and 2e.13; 1.k and 2e.14; 1.k and 2e.15; 1.k and 2e.16; 1.k and 2e.17; 1.k and 2e.18; 1.k and 2e.19; 1.k and 2e.20; 1.k and 2e.21; 1.k and 2e.22; 1.k and 2e.23; 1.k and 2e.24; 1.k and 2e.25; 1.l and 2e.1; 1.l and 2e.2; 1.l and 2e.3; 1.l and 2e.4; 1.l and 2e.5; 1.l and 2e.6; 1.l and 2e.7; 1.l and 2e.8; 1.l and 2e.9; 1.l and 2e.10; 1.l and 2e.11; 1.l and 2e.12; 1.l and 2e.13; 1.l and 2e.14; 1.l and 2e.15; 1.l and 2e.16; 1.l and 2e.17; 1.l and 2e.18; 1.l and 2e.19; 1.l and 2e.20; 1.l and 2e.21; 1.l and 2e.22; 1.l and 2e.23; 1.l and 2e.24; 1.l and 2e.25; 1.m and 2e.1; 1.m and 2e.2; 1.m and 2e.3; 1.m and 2e.4; 1.m and 2e.5; 1.m and 2e.6; 1.m and 2e.7; 1.m and 2e.8; 1.m and 2e.9; 1.m and 2e.10; 1.m and 2e.11; 1.m and 2e.12; 1.m and 2e.13; 1.m and 2e.14; 1.m and 2e.15; 1.m and 2e.16; 1.m and 2e.17; 1.m and 2e.18; 1.m and 2e.19; 1.m and 2e.20; 1.m and 2e.21; 1.m and 2e.22; 1.m and 2e.23; 1.m and 2e.24; 1.m and 2e.25; 1.q and 2e.1; 1.q and 2e.2; 1.q and 2e.3; 1.q and 2e.4; 1.q and 2e.5; 1.q and 2e.6; 1.q and 2e.7; 1.q and 2e.8; 1.q and 2e.9; 1.q and 2e.10; 1.q and 2e.11; 1.q and 2e.12; 1.q and 2e.13; 1.q and 2e.14; 1.q and 2e.15; 1.q and 2e.16, 1.q and 2e.17; 1.q and 2e.18; 1.q and 2e.19; 1.q and 2e.20; 1.q and 2e.21; 1.q and 2e.22; 1.q and 2e.23; 1.q and 2e.24 or 1.q and 2e.25, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.a, 1.h, 1.j, or 1.k, while the combinations which contain one of the compounds 1.h or 1.k are particularly important according to the invention. Of the above-mentioned combinations those which contain as compound 2e one of the compounds 2e.1, 2e.2, 2e.3, 2e.4, 2e.10, 2e.11, 2e.14, 2e.16, 2e.17, 2e.18, 2e.19, 2e.20, 2e.21, 2e.22, 2e.23, 2e.24 or 2e.25 are also preferred according to the invention, while the combinations which contain one of the compounds 2e.2, 2e.3 or 2e.4 are particularly important according to the invention.

The medicament combinations according to the invention consisting of compounds of formula 1 with at least one other active substance 2 are not limited to binary combinations of active substances. The combinations specified hereinbefore, which may contain, in addition to a compound of formula 1, another active substance 2, may also contain a third or fourth, preferably a third active substance, which is also selected from the above-mentioned group of anticholinergics (2a), PDEIV-inhibitors (2b), steroids (2c), LTD4-antagonists (2d) and EGFR-inhibitors (2e).

Particularly preferred combinations, which contain 2 further active substances in addition to a compound of formula 1 are selected from the active substance combinations listed below. These are medicament combinations, which may contain, for example:

A) a compound of formula 1, an anticholinergic (2a), a PDEIV inhibitor (2b);

B) a compound of formula 1, an anticholinergic (2a), a steroid (2c);

C) a compound of formula 1, an anticholinergic (2a), an LTD4-antagonist (2d);

D) a compound of formula 1, an anticholinergic (2a), an EGFR-inhibitor (2e);

E) a compound of formula 1, a PDEIV inhibitor (2b), a steroid (2c);

F) a compound of formula 1, a PDEIV inhibitor (2b), an LTD4-antagonist (2d);

G) a compound of formula 1, a PDEIV inhibitor (2b), an EGFR-inhibitor (2e);

H) a compound of formula 1, a steroid (2c), an LTD4-antagonist (2d);

I) a compound of formula 1, a steroid (2c), an EGFR-inhibitor (2e);

J) a compound of formula 1, an LTD4-antagonist (2d), an EGFR-inhibitor (2e).

Particularly preferred examples of medicament combinations of the above-mentioned group A are selected from among the following combinations:

compounds 1.h and 2a.1 and 2b.2; 1.h and 2a.1 and 2b.4; 1.h and 2a.1 and 2b.11; 1.h and 2a.1 and 2b.19; 1.h and 2a.9.1 and 2b.2; 1.h and 2a.9.1 and 2b.4; 1.h and 2a.9.1 and 2b.11; 1.h and 2a.9.1 and 2b.19; 1.h and 2a.9.2 and 2b.2; 1.h and 2a.9.2 and 2b.4; 1.h and 2a.9.2 and 2b.11; 1.h and 2a.9.2 and 2b.19; 1.h and 2a.10.1 and 2b.2; 1.h and 2a.10.1 and 2b.4; 1.h and 2a.10.1 and 2b.11; 1.h and 2a.10.1 and 2b.19; 1.h and 2a.10.2 and 2b.2; 1.h and 2a.10.2 and 2b.4; 1.h and 2a.10.2 and 2b.11; 1.h and 2a.10.2 and 2b.19; 1.h and 2a.11.1 and 2b.2; 1.h and 2a.11.1 and 2b.4; 1.h and 2a.11.1 and 2b.11; 1.h and 2a.11.1 and 2b.19; 1.h and 2a.11.6 and 2b.2; 1.h and 2a.11.6 and 2b.4; 1.h and 2a.11.6 and 2b.11; 1.h and 2a.11.6 and 2b.19; 1.k and 2a.1 and 2b.2; 1.k and 2a.1 and 2b.4; 1.k and 2a.1 and 2b.11; 1.k and 2a.1 and 2b.19; 1.k and 2a.9.1 and 2b.2; 1.k and 2a.9.1 and 2b.4; 1.k and 2a.9.1 and 2b.11; 1.k and 2a.9.1 and 2b.19; 1.k and 2a.9.2 and 2b.2; 1.k and 2a.9.2 and 2b.4; 1.k and 2a.9.2 and 2b.11; 1.k and 2a.9.2 and 2b.19; 1.k and 2a.10.1 and 2b.2; 1.k and 2a.10.1 and 2b.4; 1.k and 2a.10.1 and 2b.11; 1.k and 2a.10.1 and 2b.19; 1.k and 2a.10.2 and 2b.2; 1.k and 2a.10.2 and 2b.4; 1.k and 2a.10.2 and 2b.11; 1.k and 2a.10.2 and 2b.19; 1.k and 2a.11.1 and 2b.2; 1.k and 2a.11.1 and 2b.4; 1.k and 2a.11.1 and 2b.11; 1.k and 2a.11.1 and 2b.19; 1.k and 2a.11.6 and 2b.2; 1.k and 2a.11.6 and 2b.4; 1.k and 2a.11.6 and 2b.11; 1.k and 2a.11.6 and 2b.19, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group B are selected from among the following combinations:

compounds 1.h and 2a.1 and 2c.8; 1.h and 2a.1 and 2c.9; 1.h and 2a.1 and 2c.10; 1.h and 2a.1 and 2c.11; 1.h and 2a.1 and 2c.17; 1.h and 2a.9.1 and 2c.8; 1.h and 2a.9.1 and 2c.9; 1.h and 2a.9.1 and 2c.10; 1.h and 2a.9.1 and 2c.11; 1.h and 2a.9.1 and 2c.17; 1.h and 2a.9.2 and 2c.8; 1.h and 2a.9.2 and 2c.9; 1.h and 2a.9.2 and 2c.10; 1.h and 2a.9.2 and 2c.11; 1.h and 2a.9.2 and 2c.17; 1.h and 2a.10.1 and 2c.8; 1.h and 2a.10.1 and 2c.9; 1.h and 2a.10.1 and 2c.10; 1.h and 2a.10.1 and 2c.11; 1.h and 2a.10.1 and 2c.17; 1.h and 2a.10.2 and 2c.8; 1.h and 2a.10.2 and 2c.9; 1.h and 2a.10.2 and 2c.10; 1.h and 2a.10.2 and 2c.11; 1.h and 2a.10.2 and 2c.17; 1.h and 2a.11.1 and 2c.8; 1.h and 2a.11.1 and 2c.9; 1.h and 2a.11.1 and 2c.10; 1.h and 2a.11.1 and 2c.11; 1.h and 2a.11.1 and 2c.17; 1.h and 2a.11.6 and 2c.8; 1.h and 2a.11.6 and 2c.9; 1.h and 2a.11.6 and 2c.10; 1.h and 2a.11.6 and 2c.11; 1.h and 2a.11.6 and 2c.17; 1.k and 2a.1 and 2c.8; 1.k and 2a.1 and 2c.9; 1.k and 2a.1 and 2c.10; 1.k and 2a.1 and 2c.11; 1.k and 2a.1 and 2c.17; 1.k and 2a.9.1 and 2c.8; 1.k and 2a.9.1 and 2c.9; 1.k and 2a.9.1 and 2c.10; 1.k and 2a.9.1 and 2c.11; 1.k and 2a.9.1 and 2c.17; 1.k and 2a.9.2 and 2c.8; 1.k and 2a.9.2 and 2c.9; 1.k and 2a.9.2 and 2c.10; 1.k and 2a.9.2 and 2c.11; 1.k and 2a.9.2 and 2c.17; 1.k and 2a.10.1 and 2c.8; 1.k and 2a.10.1 and 2c.9; 1.k and 2a.10.1 and 2c.10; 1.k and 2a.10.1 and 2c.11; 1.k and 2a.10.1 and 2c.17; 1.k and 2a.10.2 and 2c.8; 1.k and 2a.10.2 and 2c.9; 1.k and 2a.10.2 and 2c.10; 1.k and 2a.10.2 and 2c.11; 1.k and 2a.10.2 and 2c.17; 1.k and 2a.11.1 and 2c.8; 1.k and 2a.11.1 and 2c.9; 1.k and 2a.11.1 and 2c.10; 1.k and 2a.11.1 and 2c.11; 1.k and 2a.11.1 and 2c.17; 1.k and 2a.11.6 and 2c.8; 1.k and 2a.11.6 and 2c.9; 1.k and 2a.11.6 and 2c.10; 1.k and 2a.11.6 and 2c.11; 1.k and 2a.11.6 and 2c.17, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group C are selected from among the following combinations:

compounds 1.h and 2a.1 and 2d.1; 1.h and 2a.1 and 2d.4; 1.h and 2a.1 and 2d.5; 1.h and 2a.1 and 2d.8; 1.h and 2a.9.1 and 2d.1; 1.h and 2a.9.1 and 2d.4; 1.h and 2a.9.1 and 2d.5; 1.h and 2a.9.1 and 2d.8; 1.h and 2a.9.2 and 2d.1; 1.h and 2a.9.2 and 2d.4; 1.h and 2a.9.2 and 2d.5; 1.h and 2a.9.2 and 2d.8; 1.h and 2a.10.1 and 2d.1; 1.h and 2a.10.1 and 2d.4; 1.h and 2a.10.1 and 2d.5; 1.h and 2a.10.1 and 2d.8; 1.h and 2a.10.2 and 2d.1; 1.h and 2a.10.2 and 2d.4; 1.h and 2a.10.2 and 2d.5; 1.h and 2a.10.2 and 2d.8; 1.h and 2a.11.1 and 2d.1; 1.h and 2a.11.1 and 2d.4; 1.h and 2a.11.1 and 2d.5; 1.h and 2a.11.1 and 2d.8; 1.h and 2a.11.6 and 2d.1; 1.h and 2a.11.6 and 2d.4; 1.h and 2a.11.6 and 2d.5; 1.h and 2a.11.6 and 2d.8; 1.k and 2a.1 and 2d.1; 1.k and 2a.1 and 2d.4; 1.k and 2a.1 and 2d.5; 1.k and 2a.1 and 2d.8; 1.k and 2a.9.1 and 2d.1; 1.k and 2a.9.1 and 2d.4; 1.k and 2a.9.1 and 2d.5; 1.k and 2a.9.1 and 2d.8; 1.k and 2a.9.2 and 2d.1; 1.k and 2a.9.2 and 2d.4; 1.k and 2a.9.2 and 2d.5; 1.k and 2a.9.2 and 2d.8; 1.k and 2a.10.1 and 2d.1; 1.k and 2a.10.1 and 2d.4; 1.k and 2a.10.1 and 2d.5; 1.k and 2a.10.1 and 2d.8; 1.k and 2a.10.2 and 2d.1; 1.k and 2a.10.2 and 2d.4; 1.k and 2a.10.2 and 2d.5; 1.k and 2a.10.2 and 2d.8; 1.k and 2a.11.1 and 2d.1; 1.k and 2a.11.1 and 2d.4; 1.k and 2a.11.1 and 2d.5; 1.k and 2a.11.1 and 2d.8; 1.k and 2a.11.6 and 2d.1; 1.k and 2a.11.6 and 2d.4; 1.k and 2a.11.6 and 2d.5; 1.k and 2a.11.6 and 2d.8, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group D are selected from among the following combinations:

compounds 1.h and 2a.1 and 2e.2; 1.h and 2a.1 and 2e.3; 1.h and 2a.1 and 2e.4; 1.h and 2a.1 and 2e.10; 1.h and 2a.9.1 and 2e.2; 1.h and 2a.9.1 and 2e.3; 1.h and 2a.9.1 and 2e.4; 1.h and 2a.9.1 and 2e.10; 1.h and 2a.9.2 and 2e.2; 1.h and 2a.9.2 and 2e.3; 1.h and 2a.9.2 and 2e.4; 1.h and 2a.9.2 and 2e.10; 1.h and 2a.10.1 and 2e.2; 1.h and 2a.10.1 and 2e.3; 1.h and 2a.10.1 and 2e.4; 1.h and 2a.10.1 and 2e.10; 1.h and 2a.10.2 and 2e.2; 1.h and 2a.10.2 and 2e.3; 1.h and 2a.10.2 and 2e.4; 1.h and 2a.10.2 and 2e.10; 1.h and 2a.11.1 and 2e.2; 1.h and 2a.11.1 and 2e.3; 1.h and 2a.11.1 and 2e.4; 1.h and 2a.11.1 and 2e.10; 1.h and 2a.11.6 and 2e.2; 1.h and 2a.11.6 and 2e.3; 1.h and 2a.11.6 and 2e.4; 1.h and 2a.11.6 and 2e.10; 1.k and 2a.1 and 2e.2; 1.k and 2a.1 and 2e.3; 1.k and 2a.1 and 2e.4; 1.k and 2a.1 and 2e.10; 1.k and 2a.9.1 and 2e.2; 1.k and 2a.9.1 and 2e.3; 1.k and 2a.9.1 and 2e.4; 1.k and 2a.9.1 and 2e.10; 1.k and 2a.9.2 and 2e.2; 1.k and 2a.9.2 and 2e.3; 1.k and 2a.9.2 and 2e.4; 1.k and 2a.9.2 and 2e.10; 1.k and 2a.10.1 and 2e.2; 1.k and 2a.10.1 and 2e.3; 1.k and 2a.10.1 and 2e.4; 1.k and 2a.10.1 and 2e.10; 1.k and 2a.10.2 and 2e.2; 1.k and 2a.10.2 and 2e.3; 1.k and 2a.10.2 and 2e.4; 1.k and 2a.10.2 and 2e.10; 1.k and 2a.11.1 and 2e.2; 1.k and 2a.11.1 and 2e.3; 1.k and 2a.11.1 and 2e.4; 1.k and 2a.11.1 and 2e.10; 1.k and 2a.11.6 and 2e.2; 1.k and 2a.11.6 and 2e.3; 1.k and 2a.11.6 and 2e.4; 1.k and 2a.11.6 and 2e.10, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group E are selected from among the following combinations:

compounds 1.h and 2c.8 and 2b.2; 1.h and 2c.8 and 2b.4; 1.h and 2c.8 and 2b.11; 1.h and 2c.8 and 2b.19; 1.h and 2c.9 and 2b.2; 1.h and 2c.9 and 2b.4; 1.h and 2c.9 and 2b.11; 1.h and 2c.9 and 2b.19; 1.h and 2c.10 and 2b.2; 1.h and 2c.10 and 2b.4; 1.h and 2c.10 and 2b.11; 1.h and 2c.10 and 2b.19; 1.h and 2c.11 and 2b.2; 1.h and 2c.11 and 2b.4; 1.h and 2c.11 and 2b.11; 11.h and 2c.11 and 2b.19; 1.h and 2c.17 and 2b.2; 1.h and 2c.17 and 2b.4; 1.h and 2c.17 and 2b.11; 1.h and 2c.17 and 2b.19; 1.k and 2c.8 and 2b.2; 1.k and 2c.8 and 2b.4; 1.k and 2c.8 and 2b.11; 1.k and 2c.8 and 2b.19; 1.k and 2c.9 and 2b.2; 1.k and 2c.9 and 2b.4; 1.k and 2c.9 and 2b.11; 1.k and 2c.9 and 2b.19; 1.k and 2c.10 and 2b.2; 1.k and 2c.10 and 2b.4; 1.k and 2c.10 and 2b.11; 1.k and 2c.10 and 2b.19; 1.k and 2c.11 and 2b.2; 1.k and 2c.11 and 2b.4; 1.k and 2c.11 and 2b.11; 1.k and 2c.11 and 2b.19, 1.k and 2c.17 and 2b.2; 1.k and 2c.17 and 2b.4; 1.k and 2c.17 and 2b.11; 1.k and 2c.17 and 2b.19; in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group F are selected from among the following combinations:

compounds 1.h and 2d.1 and 2b.2; 1.h and 2d.1 and 2b.4; 1.h and 2d.1 and 2b.11; 1.h and 2d.1 and 2b.19; 1.h and 2d.4 and 2b.2; 1.h and 2d.4 and 2b.4; 1.h and 2d.4 and 2b.11; 1.h and 2d.4 and 2b.19; 1.h and 2d.5 and 2b.2; 1.h and 2d.5 and 2b.4; 1.h and 2d.5 and 2b.11; 1.h and 2d.5 and 2b.19; 1.h and 2d.8 and 2b.2; 1.h and 2d.8 and 2b.4; 1.h and 2d.8 and 2b.11; 1.h and 2d.8 and 2b.19; 1.k and 2d.1 and 2b.2; 1.k and 2d.1 and 2b.4; 1.k and 2d.1 and 2b.11; 1.k and 2d.1 and 2b.19; 1.k and 2d.4 and 2b.2; 1.k and 2d.4 and 2b.4; 1.k and 2d.4 and 2b.11; 1.k and 2d.4 and 2b.19; 1.k and 2d.5 and 2b.2; 1.k and 2d.5 and 2b.4; 1.k and 2d.5 and 2b.11; 1.k and 2d.5 and 2b.19; 1.k and 2d.8 and 2b.2; 1.k and 2d.8 and 2b.4; 1.k and 2d.8 and 2b.11; 1.k and 2d.8 and 2b.19, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group G are selected from among the following combinations:

compounds 1.h and 2e.2 and 2b.2; 1.h and 2e.2 and 2b.4; 1.h and 2e.2 and 2b.11; 1.h and 2e.2 and 2b.19; 1.h and 2e.3 and 2b.2; 1.h and 2e.3 and 2b.4; 1.h and 2e.3 and 2b.11; 1.h and 2e.3 and 2b.19; 1.h and 2e.4 and 2b.2; 1.h and 2e.4 and 2b.4; 1.h and 2e.4 and 2b.11; 1.h and 2e.4 and 2b.19; 1.h and 2e.10 and 2b.2; 1.h and 2e.10 and 2b.4; 1.h and 2e.10 and 2b.11; 1.h and 2e.10 and 2b.19; 1.k and 2e.2 and 2b.2; 1.k and 2e.2 and 2b.4; 1.k and 2e.2 and 2b.11; 1.k and 2e.2 and 2b.19; 1.k and 2e.3 and 2b.2; 1.k and 2e.3 and 2b.4; 1.k and 2e.3 and 2b.11; 1.k and 2e.3 and 2b.19; 1.k and 2e.4 and 2b.2; 1.k and 2e.4 and 2b.4; 1.k and 2e.4 and 2b.11; 1.k and 2e.4 and 2b.19; 1.k and 2e.10 and 2b.2; 1.k and 2e.10 and 2b.4; 1.k and 2e.10 and 2b.11; 1.k and 2e.10 and 2b.19, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group H are selected from among the following combinations:

compounds 1.h and 2c.8 and 2d.1; 1.h and 2c.8 and 2d.4; 1.h and 2c.8 and 2d.5; 1.h and 2c.8 and 2d.8; 1.h and 2c.9 and 2d.1; 1.h and 2c.9 and 2d.4; 1.h and 2c.9 and 2d.5; 1.h and 2c.9 and 2d.8; 1.h and 2c.10 and 2d.1; 1.h and 2c.10 and 2d.4; 1.h and 2c.10 and 2d.5; 1.h and 2c.10 and 2d.8; 1.h and 2c.11 and 2d.1; 1.h and 2c.11 and 2d.4; 1.h and 2c.11 and 2d.5; 1.h and 2c.11 and 2d.8; 1.h and 2c.17 and 2d.1; 1.h and 2c.17 and 2d.4; 1.h and 2c.17 and 2d.5; 1.h and 2c.17 and 2d.8; 1.k and 2c.8 and 2d.1; 1.k and 2c.8 and 2d.4; 1.k and 2c.8 and 2d.5; 1.k and 2c.8 and 2d.8; 1.k and 2c.9 and 2d.1; 1.k and 2c.9 and 2d.4; 1.k and 2c.9 and 2d.5; 1.k and 2c.9 and 2d.8; 1.k and 2c.10 and 2d.1; 1.k and 2c.10 and 2d.4; 1.k and 2c.10 and 2d.5; 1.k and 2c.10 and 2d.8; 1.k and 2c.11 and 2d.1; 1.k and 2c.11 and 2d.4; 1.k and 2c.11 and 2d.5; 1.k and 2c.11 and 2d.8, 1.k and 2c.17 and 2d.1; 1.k and 2c.17 and 2d.4; 1.k and 2c.17 and 2d.5; 1.k and 2c.17 and 2d.8; in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group I are selected from among the following combinations:

compounds 1.h and 2c.8 and 2e.2; 1.h and 2c.8 and 2e.3; 1.h and 2c.8 and 2e.4; 1.h and 2c.8 and 2e.10; 1.h and 2c.9 and 2e.2; 1.h and 2c.9 and 2e.3; 1.h and 2c.9 and 2e.4; 1.h and 2c.9 and 2e.10; 1.h and 2c.10 and 2e.2; 1.h and 2c.10 and 2e.3; 1.h and 2c.10 and 2e.4; 1.h and 2c.10 and 2e.10; 1.h and 2c.11 and 2e.2; 1.h and 2c.11 and 2e.3; 1.h and 2c.11 and 2e.4; 1.h and 2c.11 and 2e.10; 1.h and 2c.17 and 2e.2; 1.h and 2c.17 and 2e.3; 1.h and 2c.17 and 2e.4; 1.h and 2c.17 and 2e.10; 1.k and 2c.8 and 2e.2; 1.k and 2c.8 and 2e.3; 1.k and 2c.8 and 2e.4; 1.k and 2c.8 and 2e.10; 1.k and 2c.9 and 2e.2; 1.k and 2c.9 and 2e.3; 1.k and 2c.9 and 2e.4; 1.k and 2c.9 and 2e.10; 1.k and 2c.10 and 2e.2; 1.k and 2c.10 and 2e.3; 1.k and 2c.10 and 2e.4; 1.k and 2c.10 and 2e.10; 1.k and 2c.11 and 2e.2; 1.k and 2c.11 and 2e.3; 1.k and 2c.11 and 2e.4; 1.k and 2c.11 and 2e.10, 1.k and 2c.17 and 2e.2; 1.k and 2c.17 and 2e.3; 1.k and 2c.17 and 2e.4; 1.k and 2c.17 and 2e.10; in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group J are selected from among the following combinations:

compounds 1.h and 2d.1 and 2e.2; 1.h and 2d.1 and 2e.3; 1.h and 2d.1 and 2e.4; 1.h and 2d.1 and 2e.10; 1.h and 2d.4 and 2e.2; 1.h and 2d.4 and 2e.3; 1.h and 2d.4 and 2e.4; 1.h and 2d.4 and 2e.10; 1.h and 2d.5 and 2e.2; 1.h and 2d.5 and 2e.3; 1.h and 2d.5 and 2e.4; 1.h and 2d.5 and 2e.10; 1.h and 2d.8 and 2e.2; 1.h and 2d.8 and 2e.3; 1.h and 2d.8 and 2e.4; 1.h and 2d.8 and 2e.10; 1.k and 2d.1 and 2e.2; 1.k and 2d.1 and 2e.3; 1.k and 2d.1 and 2e.4; 1.k and 2d.1 and 2e.10; 1.k and 2d.4 and 2e.2; 1.k and 2d.4 and 2e.3; 1.k and 2d.4 and 2e.4; 1.k and 2d.4 and 2e.10; 1.k and 2d.5 and 2e.2; 1.k and 2d.5 and 2e.3; 1.k and 2d.5 and 2e.4; 1.k and 2d.5 and 2e.10; 1.k and 2d.8 and 2e.2; 1.k and 2d.8 and 2e.3; 1.k and 2d.8 and 2e.4; 1.k and 2d.8 and 2e.10, in each case optionally in the form of the racemates, enantiomers or diastereomers thereof and optionally in the form of the pharmacologically acceptable acid addition salts, solvates and/or hydrates thereof.

Of outstanding importance according to the invention are all the medicament combinations disclosed within the scope of the present invention which contain the compounds of formula 1 in the form of their R-enantiomers.

Halogen within the scope of the present invention denotes fluorine, chlorine, bromine or iodine. Unless stated otherwise, fluorine and chlorine are the preferred halogens, while fluorine is generally preferred.

Unless otherwise stated, the alkyl groups (alkyl) are straight-chained or branched alkyl groups having 1 to 6, preferably 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the alkylene groups (alkylene) are branched and unbranched alkylene groups with 1 to 6, preferably 1 to 4 carbon atoms. The following are mentioned by way of example: methylene, ethylene, propylene or butylene. Unless otherwise stated, the definitions propylene and butylene include all the possible isomeric forms of the groups in question.

Unless otherwise stated, the cycloalkyl groups (cycloalkyl) are cyclic alkyl groups with 3 to 6. The following are mentioned by way of example: cyclopropyl, cyclobutanyl, cyclopentyl or cyclohexyl.

Unless otherwise stated, the alkyloxy groups (O-alkyl) are branched and unbranched alkyl groups with 1 to 6, preferably 1 to 4 carbon atoms which are linked via an oxygen atom. The following are mentioned by way of example: methyloxy, ethyloxy, propyloxy or butyloxy. In some cases the abbreviations —OMe, —OEt, —OProp or —OBu may be used to denote the methyloxy, ethyloxy, propyloxy or butyloxy groups. Unless otherwise stated, the definitions propyloxy and butyloxy include all the possible isomeric forms of the groups in question. Thus, for example, propyloxy includes n-propyloxy and iso-propyloxy, butyloxy includes iso-butyloxy, sec.butyloxy and tert.-butyloxy, etc. In some cases the term alkoxy may be used instead of alkyloxy within the scope of the present invention. The groups methyloxy, ethyloxy, propyloxy or butyloxy may therefore also be referred to by the names methoxy, ethoxy, propoxy or butoxy.

Unless otherwise stated, the haloalkylene groups (haloalkyl) are branched and unbranched alkyl groups with 1 to 6 carbon atoms, wherein one or more hydrogen atoms are replaced by halogen atoms, preferably by fluorine. Examples include: CHF₂, CF₃, CH₂CF₃, CF₂CF₃.

Unless otherwise stated, the aryl groups are aromatic ring systems with 6 to 10 carbon atoms. Preferred aryl groups are phenyl and naphthyl, while phenyl is particularly preferred according to the invention.

Unless otherwise stated, the arylalkylene groups are the above-mentioned aryl groups which are linked by branched and unbranched alkyl groups with 1 to 4 carbon atoms. Examples include benzyl, phenylethyl, naphthylmethyl, naphthylethyl. The bridging alkyl groups are also referred to as alkylene bridges within the scope of the present invention.

Unless otherwise stated, the aryloxy groups (O-aryl) are aryl groups with 6 to 10 carbon atoms, which are linked by an oxygen bridge. Preferred groups in this context are for example phenyloxy or naphthyloxy, which may also be referred to as phenoxy or naphthoxy within the scope of the present invention.

Unless otherwise stated, the arylalkylenoxy groups (arylalkylene-O—) are aryl groups which are linked by branched and unbranched alkyloxy groups with 1 to 4 carbon atoms. Examples include benzyloxy, phenylethyloxy, naphthylmethyloxy, naphthylethyloxy.

Within the scope of the present invention the expression medicament combination of components 1 and 2 denotes the joint administration of both active substances in a single preparation or formulation or the separate administration of the two active substances in separate formulations. If the active substances 1 and 2 are administered in separate formulations, this separate administration may be carried out simultaneously or at staggered times, i.e. sequentially.

In one aspect the present invention relates to the above-mentioned medicament combinations which contain in addition to therapeutically effective amounts of 1 and 2 a pharmaceutically acceptable carrier. In one aspect the present invention relates to the above-mentioned pharmaceutical compositions which do not contain a pharmaceutically acceptable carrier in addition to therapeutically effective amounts of 1 and 2.

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the active substance 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

The present invention also relates to the use of therapeutically effective amounts of an active substance of formula 1 in combination with therapeutically effective amounts of active substance 2 for preparing a pharmaceutical composition for the treatment of one of the above-mentioned diseases.

The present invention also relates to a process for treating one of the above-mentioned diseases, which is characterised in that therapeutically effective amounts of an active substance of formula 1 are administered in combination with therapeutically effective amounts of an active substance 2.

Within the scope of the medicament combinations according to the invention, for example, 0.1-1000 μg of a compound of formula 1 may be administered per single dose. Preferably, 1-500 μg, particularly preferably 3-100 μg of the compound of formula 1 are administered per single dose, while a dosage range of from 5-75 μg, preferably from 7-50 μg is preferred according to the invention. Particularly preferably, the pharmaceutical compositions according to the invention are administered in an amount such that 9-40 μg, particularly preferably 11-30 μg, more preferably 12-25 μg of the compound of formula 1 are administered per single dose. For example, and without restricting the present invention thereto, 5 μg, 7.5 μg, 10 μg, 12.5 μg, 15 μg, 17.5 μg, 20 μg, 22.5 μg, 25 μg, 27.5 μg, 30 μg, 32.5 μg, 35 μg, 37.5 μg, 40 μg, 42.5 μg, 45 μg, 47.5 μg, 50 μg, 52.5 μg, 55 μg, 57.5 μg, 60 μg, 62.5 μg, 65 μg, 67.5 μg, 70 μg, 72.5 μg or 75 μg of a compound of formula 1 may be administered per single dose.

The above-mentioned dosages relate to the compounds of formula 1 in the form of their free bases. If the compounds of formula 1 are administered in the form of their pharmaceutically acceptable acid addition salts, the skilled man can easily calculate the corresponding dosage ranges for the acid addition salts from the dosage ranges specified above, taking into account the molecular weight of the acids used. Particularly preferably, the compounds of formula 1 are administered in the above-mentioned dosage ranges in the form of the enantiomerically pure compounds, particularly preferably in the form of the R-enantiomers thereof.

If the compounds of formula 1 are administered in conjunction with an anticholinergic 2a, the amount of anticholinergic used will fluctuate considerably depending on the choice of active substance.

Without restricting the invention thereto, in the case of tiotropium 2a.1′ amounts of anticholinergic (2a.1′) may be administered such that each single dose contains 0.1-80 μg, preferably 0.5-60 μg, particularly preferably about 1-50 μg of 2a.1′. For example and without restricting the present invention thereto, 2.5 μg, 5 μg, 10 μg, 18 μg, 20 μg, 36 μg or 40 μg 2a.1′ may be administered per single dose. The corresponding amount of salt 2a.1 or of any hydrate or solvate used in each case can easily be calculated by the skilled man, depending on the choice of anion. If for example tiotropium bromide is used as the preferred tiotropium salt 2a.1 according to the invention, the amounts of the active substance 2a.1′ administered per single dose as specified by way of example hereinbefore correspond to the following amounts of 2a.1 administered per single dose: 3 μg, 6 μg, 12 μg, 21.7 μg, 24.1 μg, 43.3 μg and 48.1 μg 2a.1. In the case of tiotropium 2a.1′ the dosages specified above are preferably administered once or twice a day, while administration once a day is particularly preferred according to the invention

Without restricting the invention thereto, in the case of the cation 2a.2′ amounts of anticholinergic (2a.2′) may be administered such that each single dose contains 1-500 μg, preferably 5-300 μg, particularly preferably 15-200 μg 2a.2′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 kg of 2a.2′ may be administered per single dose. The corresponding amount of salt 2a.2 used in each case or of any hydrate or solvate used can easily be calculated by the skilled man, depending on the choice of anion. In the case of oxitropium 2a.2′ the dosages specified above are preferably administered one to four times a day, while administration two to three times a day is particularly preferred according to the invention.

Without restricting the invention thereto, in the case of the cation 2a.3′ amounts of anticholinergic (2a.3′) may be administered such that each single dose contains 1-500 μg, preferably 5-300 μg, particularly preferably 15-200 μg 2a.3′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.3′ may be administered per single dose. The corresponding amount of salt 2a.3 used in each case or of any hydrate or solvate used can easily be calculated by the skilled man, depending on the choice of anion. In the case of flutropium 2a.3′ the dosages specified above are preferably administered one to four times a day, while administration two to three times a day is particularly preferred according to the invention.

Without restricting the invention thereto, in the case of the cation 2a.4′ amounts of anticholinergic (2a.4′) may be administered such that each single dose contains 1-500 μg, preferably 5-300 μg, particularly preferably 20-200 μg 2a.4′. For example and without restricting the present invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 10 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.4′ may be administered per single dose. The corresponding amount of salt 2a.4 used in each case or of any hydrate or solvate used can easily be calculated by the skilled man, depending on the choice of anion. In the case of ipratropium 2a.4′ the dosages specified above are preferably administered one to four times a day, while administration two to three times a day, more preferably three times a day, is particularly preferred according to the invention.

Without restricting the invention thereto, in the case of the cation 2a.5′ amounts of anticholinergic (2a.5′) may be administered such that each single dose contains 1-500 μg, preferably 5-300 μg, particularly preferably 15-200 μg. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 kg of 2a.5′ may be administered per single dose. The corresponding amount of salt 2a.5 used in each case or of any hydrate or solvate used can easily be calculated by the skilled man, depending on the choice of anion. In the case of glycopyrronium 2a.5′ the dosages specified above are preferably administered one to four times a day, while administration two to three times a day is particularly preferred according to the invention.

Without restricting the invention thereto, in the case of the cation 2a.6′ amounts of anticholinergic (2a.6′) may be administered such that each single dose contains 1000-6500 μg, preferably 2000-6000 μg, particularly preferably 3000-5500 μg, particularly preferably 4000-5000 μg 2a.6′. For example and without restricting the present invention thereto, 3500 μg, 3750 μg, 4000 μg, 4250 μg, 4500 μg, 4750 μg, or 5000 μg of 2a.6′ may be administered per single dose. The corresponding amount of salt 2a.6 used in each case or of any hydrate or solvate used can easily be calculated by the skilled man, depending on the choice of anion. In the case of trospium 2a.6′ the dosages specified above are preferably administered one to four times a day, while administration two to three times a day is particularly preferred according to the invention.

Without restricting the invention thereto, in the case of the cation 2a.7′ amounts of anticholinergic (2a.7′) may be administered such that each single dose contains 50-1000 μg, preferably 100-800 μg, particularly preferably 200-700 μg, particularly preferably 300-600 μg 2a.7′. For example and without restricting the present invention thereto, 300 μg, 350 μg, 400 μg, 450 μg, 500 μg, 550 μg, or 600 μg of 2a.7′ may be administered per single dose. The corresponding amount of salt 2a.7 used in each case or of any hydrate or solvate used can easily be calculated by the skilled man, depending on the choice of anion. In the case of the cation 2a.7′ the dosages specified above are preferably administered one to three times a day, while administration once or twice a day, more preferably once a day, is particularly preferred according to the invention.

Without restricting the invention thereto, in the case of the cations 2a.9′ and 2a.10′, amounts of anticholinergic (2a.9′ or 2a.10′) may be administered such that each single dose contains 1-500 μg, preferably 5-300 μg, particularly preferably 15-200 μg 2a.9′ or 2a.10′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.9′ or 2a.10′ may be administered per single dose. The corresponding amount of salt 2a.9′ or 2a.10′ or of any hydrate or solvate used in each case can easily be calculated by the skilled man, depending on the choice of anion. In the case of the cations 2a.9′ or 2a.10′ the dosages specified above are preferably administered one to three times a day, while administration once or twice a day, more preferably once a day, is particularly preferred according to the invention.

Without restricting the invention thereto, in the case of the cations 2a.11′ to 2a.13′ amounts of anticholinergic (2a.11′, 2a.12′ or 2a.13′) may be administered such that each single dose contains 1-500 μg, preferably 5-300 μg, particularly preferably 10-200 μg 2a.11′, 2a.12′ or 2a.13′. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.11′, 2a.12′ or 2a.13′ may be administered per single dose. The corresponding amount of salt 2a.11, 2a.12 or 2a.13 or of any hydrate or solvate used in each case can easily be calculated by the skilled man, depending on the choice of anion. In the case of the cations 2a.11, 2a.12 or 2a.13 the dosages specified above are preferably administered one to three times a day, while administration once or twice a day, more preferably once a day, is particularly preferred according to the invention.

If the compounds of formula 1 are administered in combination with a PDE IV-inhibitor 2b, preferably about 1-10000 μg 2b are administered per single dose. Preferably, amounts of 2b are administered such that each single dose contains 10-5000 μg, preferably 50-2500 μg, particularly preferably 100-1000 μg of 2b. For example and without restricting the present invention thereto, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 kg, 205 μg, 210 kg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 kg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 kg, 605 μg, 610 kg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 kg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2b may be administered per single dose. In the event that acid addition salts of 2b are used, the corresponding amount of salt used can easily be calculated by the skilled man from the values given hereinbefore, depending on the choice of acid.

If the compounds of formula 1 are administered in combination with a steroid 2c, preferably about 1-10000 μg of 2c are administered per single dose. Preferably, amounts of 2c are administered such that each single dose contains 5-5000 μg, preferably 5-2500 μg, particularly preferably 10-1000 μg of 2c. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2c may be administered per single dose. In the event that salts or derivatives of 2c are used, the corresponding amount of salt/derivative used can easily be calculated by the skilled man from the values given hereinbefore, depending on the choice of salt/derivative.

If the compounds of formula 1 are administered in combination with an LTD4-antagonist 2d, preferably about 0.01-500 mg 2d are administered per single dose. Preferably, amounts of 2d are administered such that each single dose contains 0.1-250 mg, preferably 0.5-100 mg, particularly preferably 1-50 mg of 2d. For example and without restricting the present invention thereto, 1 mg, 2.5 mg, 5 mg, 5.5 mg, 7 mg, 7, 5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg or 50 mg of 2d may be administered per single dose. In the event that acid addition salts, salts or derivatives of 2d are used, the corresponding amount of salt/derivative used can easily be calculated by the skilled man from the values given hereinbefore, depending on the choice of salt/derivative.

If the compounds of formula 1 are administered in combination with an EGFR-inhibitor 2e, preferably about 100-15000 μg of 2e are administered per single dose. Preferably, amounts of 2e are administered such that each single dose contains 500-10000 μg, preferably 750-8000 μg, particularly preferably 1000-7000 μg of 2e. For example and without restricting the present invention thereto, 1000 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3450 μg, 3500 μg, 3550 μg, 3600 μg, 3650 μg, 3700 μg, 3750 μg, 3800 μg, 3850 μg, 3900 μg, 3950 μg, 4000 μg, 4050 μg, 4100 μg, 4150 μg, 4200 μg, 4250 μg, 4300 μg, 4350 μg, 4400 μg, 4450 μg, 4500 μg, 4550 μg, 4600 μg, 4650 μg, 4700 μg, 4750 μg, 4800 μg, 4850 μg, 4900 μg, 4950 μg, 5000 μg, 5050 μg, 5100 μg, 5150 μg, 5200 μg, 5250 μg, 5300 μg, 5350 μg, 5400 μg, 5450 μg, 5500 μg, 5550 μg, 5600 μg, 5650 μg, 5700 μg, 5750 μg, 5800 μg, 5850 μg, 5900 μg, 5950 μg, 6000 μg, 6050 μg, 6100 μg, 6150 μg, 6200 μg, 6250 μg, 6300 μg, 6350 μg, 6400 μg, 6450 μg, 6500 μg, 6550 μg, 6600 μg, 6650 μg, 6700 μg, 6750 μg, 6800 μg, 6850 μg, 6900 μg, 6950 μg, or 7000 μg of 2e may be administered per single dose. In the event that acid addition salts of 2e are used, the corresponding amount of the salt used can easily be calculated by the skilled man from the values given hereinbefore, depending on the choice of acid.

The two active substance components 1 and 2 may be administered—together or separately—in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

Suitable preparations for administering the compounds of formula 1 and 2 include tablets, capsules, suppositories, solutions, powders, etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

Preferably, even when the two components 1 and 2 are administered separately, at least component 1 is administered by inhalation. If component 1 is administered by inhalation, when the two active substances are taken separately, component 2 may also be administered for example by oral or parenteral route using formulations conventional in the art such as plain or coated tablets, pills, granules, aerosols, syrups, emulsions, suspensions, powders and solutions, using inert, non-toxic, pharmaceutically suitable carriers or solvents.

Preferably, however, the medicament combinations according to the invention are administered by inhalation by means of a single preparation containing both active substances 1 and 2 or by means of separate preparations each containing only one of the active substances 1 and 2, suitable for administration by inhalation.

Inhalable preparations include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances According to the Invention:

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbuhaler® or using inhalers as disclosed for example in EP 237507 A. Preferably, the inhalable powders according to the invention which contain physiologically acceptable excipient in addition to 1 and 2 are packed into capsules (to produce so-called inhalettes) which are used in inhalers as described, for example, in WO 94/28958.

A particularly preferred inhaler for using the pharmaceutical combination according to the invention in inhalettes is shown in FIG. 1.

This inhaler (Handihaler®) for inhaling powdered pharmaceutical compositions from capsules is characterised by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

B) Propellant Gas-Driven Inhalation Aerosols Containing the Combinations of Active Substances According to the Invention:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

The above-mentioned examples of the active substances 2 are known in the art. The compounds of formula 1 by contrast are not known in the art.

The examples of synthesis described hereinafter serve to illustrate possible methods of synthesising the new compounds of formula 1. However, they are intended only as examples of procedures as an illustration of the invention without restricting the invention to the subject-matter described by way of example.

### EXAMPLE 1

### 1-{3-[2-hydroxy-2-(6-hydroxy-3-oxo-3,4-dihydro-2H-benzo[1,4]oxazin-8-yl)-ethylamino]-3-methyl-butyl}-6-methoxy-4,4-dimethyl-1,4-dihydro-benzo[d][1,3]oxazin-2-one

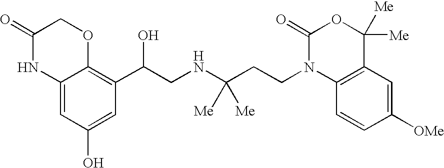

### a) ethyl (2-acetyl-4-methoxy-phenyl)-carbamate

65.1 g (0.6 mol) ethyl chloroformate are added dropwise, with cooling, to a solution of 82.5 g (0.5 mol) 2-amino-5-methoxyacetophenone in 400 mL pyridine, such that the temperature does not exceed 10-15° C. Then the reaction mixture is stirred for 2 h at ambient temperature and then poured onto ice. The precipitate formed is suction filtered, washed with water and recrystallised from isopropanol. Yield: 102 g (86%); m.p.=97-100° C.

### b) 6-methoxy-4,4-dimethyl-1,4-dihydro-benzo[1,3]oxazin-2-one

47.4 g (0.2 mol) ethyl (2-acetyl-4-methoxy-phenyl)-carbamate, dissolved in 275 mL THF, are added dropwise, while being cooled, to a solution of 0.5 mol methylmagnesium iodide in 200 mL diethyl ether such that the temperature does not exceed 0° C. The mixture is left for 30 minutes at ambient temperature and then refluxed for 2 hours with stirring. The reaction mixture is poured onto ice and combined with ammonium chloride. After separation of the organic phase the mixture is repeatedly extracted with ethyl acetate. The organic phases are combined, washed with water, dried with sodium sulphate and concentrated by evaporation. The residue is dissolved in methanol and the solution is concentrated by evaporation and then combined with water. The precipitate formed is separated off, washed with water and recrystallised from toluene. Yield: 31.1 g (75%); m.p.=178-180° C.

### c) 1-(3-amino-3-methyl-butyl)-6-methoxy-4,4-dimethyl-1,4-dihydro-benzo[1,3]oxazin-2-one

A solution of 31 g (0.15 mol) 6-methoxy-4,4-dimethyl-1,4-dihydro-benzo[1,3]oxazin-2-one in 120 mL HMPT is added dropwise at 65-70° C. to 7.2 g sodium hydride (55-60%) in 30 mL HMPT. After the release of hydrogen has stopped the mixture is stirred for another 20 minutes and then cooled to ambient temperature. At this temperature 37.7 g (0.18 mol) (3-chloro-1,1-dimethyl-propyl)-benzylideneamine, dissolved in 40 mL HMPT, are added. after 3 hours' stirring at 100° C. the reaction mixture is poured onto ice and extracted with ethyl acetate. The organic phases are washed with water, dried with sodium sulphate and concentrated by evaporation. The residue is dissolved in 1 N hydrochloric acid with heating and after cooling extracted with diethyl ether. The aqueous phase is made alkaline with sodium hydroxide solution and extracted with ethyl acetate. Then the organic phase is dried with sodium sulphate and freed from the solvent. The product is isolated from the residue in the form of its hydrochloride after dissolving in acetonitrile and adding ethereal hydrochloric acid. Yield: 34.3 g (70%).

### d) 1-{3-[2-(6-benzyloxy-3-oxo-3,4-dihydro-2H-benzo[1,4]oxazin-8-yl)-2-hydroxy-ethylamino]-3-methyl-butyl}-6-methoxy-4,4-dimethyl-1,4-dihydro-benzo[1,3]oxazin-2-one

357 mg (1 mmol) 6-benzyloxy-8-(2-ethoxy-2-hydroxy-acetyl)-4H-benzo[1,4]oxazin-3-one and 292 mg (1 mmol) 1-(3-amino-3-methyl-butyl)-6-methoxy-4,4-dimethyl-1,4-dihydro-benzo[1,3]oxazin-2-one are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for another 30 minutes. It is filtered through aluminium oxide (basic), washed repeatedly with methylene chloride/methanol 15:1 and concentrated by evaporation. The crude product thus obtained is purified by chromatography (methylene chloride with methanol/ammonia gradient (9:1)). Beige solid. Yield: 340 mg (58%); mass spectrometry: [M+H]⁺=590.

### e) 1-{3-[2-hydroxy-2-(6-hydroxy-3-oxo-3,4-dihydro-2H-benzo[1,4]oxazin-8-yl)-ethylamino]-3-methyl-butyl}-6-methoxy-4,4-dimethyl-1,4-dihydro-benzod[d][1,3]oxazin-2-one

340 mg (0.58 mmol) 1-{3-[2-(6-benzyloxy-3-oxo-3,4-dihydro-2H-benzo[1,4]oxazin-8-yl)-2-hydroxy-ethylamino]-3-methyl-butyl}-6-methoxy-4,4-dimethyl-1,4-dihydro-benzo[1,3]oxazin-2-one are dissolved in 10 mL methanol and hydrogenated with palladium on charcoal as catalyst at 1 bar hydrogen pressure. Then the catalyst is filtered off and the filtrate is concentrated by evaporation. Beige solid. Yield: 273 mg (95%); mass spectrometry: [M+H]⁺=500; Rf value=0.33 (methylene chloride:methanol: ammonia=9:1:0.1).

### EXAMPLE 2

### 6-hydroxy-1-{3-[2-hydroxy-2-(6-hydroxy-3-oxo-3,4-dihydro-2H-benzo[1,4]oxazin-8-yl)-ethylamino]-3-methyl-butyl}-4,4-dimethyl-1,4-dihydro-benzo[d][1,3]oxazin-2-one

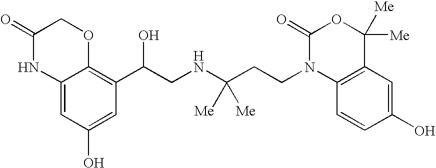

### a) 6-benzyloxy-4,4-dimethyl-1,4-dihydro-benzo[1,3]oxazin-2-one

Prepared analogously to the procedure laid down for Example 1b) from 15.7 g (50 mmol) ethyl (2-acetyl-4-benzyloxy-phenyl)-carbamate and 125 mmol methylmagnesium iodide. Yield: 10.8 g (76%); m.p.=134° C.

### b) 1-(3-amino-3-methyl-butyl)-6-benzyloxy-4,4-dimethyl-1,4-dihydro-benzo[1,3]oxazin-2-one

Prepared analogously to the procedure laid down for Example 1c) from 10.5 g (37 mmol) 6-benzyloxy-4,4-dimethyl-1,4-dihydro-benzo[1,3]oxazin-2-one and 9.3 g (44 mmol) (3-chloro-1,1-dimethyl-propyl)-benzylideneamine. Yield: 10.9 g (73%); m.p.=233° C. (hydrochloride).

### c) 6-hydroxy-1-{3-[2-hydroxy-2-(6-hydroxy-3-oxo-3,4-dihydro-2H-benzo[1,4]oxazin-8-yl)-ethylamino]-3-methyl-butyl}-4,4-dimethyl-1,4-dihydro-benzo[1,3]oxazin-2-one

The reaction of 357 mg (1 mmol) 6-benzyloxy-8-(2-ethoxy-2-hydroxy-acetyl)-4H-benzo[1,4]oxazin-3-one and 368 mg (1 mmol) 1-(3-amino-3-methyl-butyl)-6-benzyloxy-4,4-dimethyl-1,4-dihydro-benzo[1,3]oxazin-2-one analogously to the procedures laid down for Example 1 yields the compound in the form of a beige solid. Yield 355 mg (73%); mass spectrometry: [M+H]⁺=486.

### EXAMPLE 3

### 4,4-diethyl-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[d][1,3]oxazin-2-one

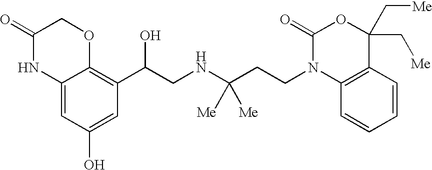

### a) 4,4-diethyl-1,4-dihydro-benzo[1,3]oxazin-2-one

Obtained from reacting 67 g (0.3 mol) methyl 2-ethoxycarbonylamino-benzoate and 1.14 mol ethylmagnesium iodide analogously to the procedure laid down for Example 1b). Yield: 48.5 g (79%); m.p.=160-162° C.

### b) 1-(3-amino-3-methyl-butyl)-4,4-diethyl-1,4-dihydro-benzo[1,3]oxazin-2-one

Prepared from 47.5 g (0.23 mol) 4,4-diethyl-1,4-dihydro-benzo[1,3]oxazin-2-one and 57.5 g (0.27 mol) (3-chloro-1,1-dimethyl-propyl)-benzylideneamine by the method described for Example 1c). Yield: 38.1 g (50%); m.p.=208-210° C. (hydrochloride).

### c) 4,4-diethyl-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[d][1,3]oxazin-2-one

357 mg (1 mmol) 6-benzyloxy-8-(2-ethoxy-2-hydroxy-acetyl)-4H-benzo[1,4]oxazin-3-one and 290 mg (1 mmol) 1-(3-amino-3-methyl-butyl)-4,4-diethyl-1,4-dihydro-benzo[1,3]oxazin-2-one are reacted analogously to the methods laid down for Example 1. After subsequent debenzylation a beige solid is obtained. Yield: 367 mg (74%); mass spectrometry: [M+H]⁺=498.

### EXAMPLE 4

### 1-{3-[2-hydroxy-2-(6-hydroxy-3-oxo-3,4-dihydro-2H-benzo[1,4]oxazin-8-yl)-ethylamino]-3-methyl-butyl}-4,4-dimethyl-1,4-dihydro-benzo[d][1,3]oxazin-2-one

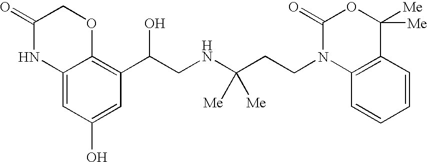

### a) 4,4-dimethyl-1,4-dihydro-benzo[1,3]oxazin-2-one

112 g (1.13 mol) phosgene are piped into 500 mL THF. Then a solution of 52 g (0.34 mol) 2-(2-amino-phenyl)-propan-2-ol, prepared from 2-aminoacetophenone and methylmagnesium iodide, in 300 mL THF is added. The reaction mixture is left to stand overnight, concentrated by evaporation and combined with 500 ml of pyridine. After the pyridine has been distilled off the remainder is combined with water and extracted with diethyl ether. The organic phases are washed successively with 2 N hydrochloric acid, sodium hydroxide solution and water, dried with sodium sulphate and concentrated by evaporation. The residue remaining (46 g) is further reacted directly, without any more purification. M.p. (toluene/petroleum ether)=109-110° C.

### b) 1-(3-amino-3-methyl-butyl)-4,4-dimethyl-1,4-dihydro-benzo[1,3]oxazin-2-one

Obtained from 43 g (0.24 mol) 4,4-dimethyl-1,4-dihydro-benzo[1,3]oxazin-2-one and 54 g (0.26 mol) (3-chloro-1,1-dimethyl-propyl)-benzylideneamine analogously to the methods described for Example 1c). Yield 41 g (57%); m.p. (after recrystallisation from ethanol)=262° C. (hydrochloride).

### c) 1-{3-[2-hydroxy-2-(6-hydroxy-3-oxo-3,4-dihydro-2H-benzo[1,4]oxazin-8-yl)-ethylamino]-3-methyl-butyl}-4,4-dimethyl-1,4-dihydro-benzo[d][1,3]oxazin-2-one

357 mg (1 mmol) 6-benzyloxy-8-(2-ethoxy-2-hydroxy-acetyl)-4H-benzo[1,4]oxazin-3-one and 262 mg (1 mmol) 1-(3-amino-3-methyl-butyl)-4,4-dimethyl-1,4-dihydro-benzo[1,3]oxazin-2-one are reacted as described for Example 1. After subsequent hydrogenation a beige solid is isolated. Yield: 285 mg (61%); mass spectrometry [M+H]⁺=470.

### EXAMPLE 5

### 1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[1,3]oxazin-2-one

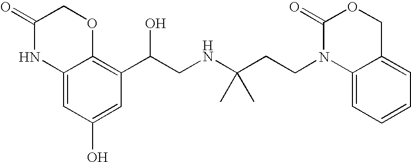

### a) 1-(3-amino-3-methyl-butyl)-1,4-dihydro-benzo[1,3]oxazin-2-one

2.70 g (18 mmol) 1,4-dihydro-benzo[1,3]oxazin-2-one and 4.35 g (21 mmol) (3-chloro-1,1-dimethyl-propyl)-(1-phenyl-methylidene)-amine are reacted as described for Example 6a). For working up the reaction mixture is poured onto ice water and extracted with ethyl acetate. The organic phases are washed with water, dried with sodium sulphate and concentrated by evaporation. The residue is combined with 25 mL 2 N hydrochloric acid and heated to 70° C. After cooling to ambient temperature the mixture is extracted with diethyl ether. The aqueous phase is concentrated by evaporation and combined with acetonitrile. The precipitate formed is suction filtered and washed with acetonitrile and diethyl ether. Yield: 2.65 g (54%, hydrochloride); melting range: 220° C. (decomposition).

### b) 1-{3-[2-(6-benzyloxy-3-oxo-3,4-dihydro-2H-benzo[1,4]oxazin-8-yl)-2-hydroxy-ethylamino]-3-methyl-butyl}-1,4-dihydro-benzo[1,3]oxazin-2-one

357 mg (1 mmol) 6-benzyloxy-8-(2-ethoxy-2-hydroxy-acetyl)-4H-benzo[1,4]oxazin-3-one and 234 mg (1 mmol) 1-(3-amino-3-methyl-butyl)-1,4-dihydro-benzo[1,3]oxazin-2-one in mL tetrahydrofuran are stirred for 15 minutes at 60° C. The mixture is cooled to 0° C. and under an argon atmosphere 1.5 mL of a 2 molar solution of lithium borohydride in tetrahydrofuran is added dropwise. The mixture is stirred for 15 min at 0° C., combined with mL dichloromethane and 3 mL water, stirred for another hour and then filtered through kieselguhr. The mixture is eluted with dichloromethane and the solvents are distilled off. The residue is purified by preparative HPLC (reverse phase, acetonitrile/water gradient with 0.1% trifluoroacetic acid). Yield: 196 mg (30%, trifluoroacetate); mass spectroscopy: [M]⁺=532.

### c) 1-{3-[2-(6-benzyloxy-3-oxo-3,4-dihydro-2H-benzo[1,4]oxazin-8-yl)-2-hydroxy-ethylamino]-3-methyl-butyl}-1,4-dihydro-benzo[1,3]oxazin-2-one

196 mg (0.3 mmol) of 1-{3-[2-(6-benzyloxy-3-oxo-3,4-dihydro-2H-benzo[1,4]oxazin-8-yl)-2-hydroxy-ethylamino]-3-methyl-butyl}-1,4-dihydro-benzo[1,3]oxazin-2-one are dissolved in 5 ethanol and hydrogenated with palladium on charcoal (10%) as catalyst at 3 bar and at ambient temperature. The catalyst is separated off and the crude product is recrystallised from acetonitrile/diethyl ether. Yield: 48 mg (29%, trifluorethyl acetate); mass spectroscopy: [M+H]⁺=442.

### EXAMPLE 6

### 4-ethyl-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[1,3]oxazin-2-one

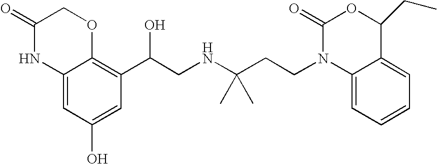

### a) 1-(3-amino-3-methyl-butyl)-4-ethyl-1,4-dihydro-benzo[1,4]oxazine

A solution of 17.7 g (0.10 mol) 4-ethyl-1,4-dihydro-benzo[1,3]oxazin-2-one in 85 mL HMPT is combined with 4.8 g sodium hydride (55-60%) and slowly heated to 60° C. After the development of hydrogen has ended the mixture is stirred for another 30 min at 80° C. and then cooled to ambient temperature. 25 g (0.12 mol) (3-chloro-1,1-dimethyl-propyl)-(1-phenyl-methylidene)-amine, dissolved in 25 mL HMPT, are added and the mixture is stirred for three hours at 100° C. The reaction mixture is cooled, poured onto ice water and extracted with ethyl acetate. The combined organic phases are washed with water, dried with sodium sulphate and concentrated by evaporation. The residue is heated to 60° C. with 240 mL 1N hydrochloric acid and after cooling extracted with diethyl ether. The aqueous phase is made alkaline with conc. sodium hydroxide solution and extracted with ethyl acetate. The combined organic phases are dried with sodium sulphate and concentrated by evaporation. The residue is dissolved in ethyl acetate with heating, combined with an equimolar amount of maleic acid and slowly cooled. The precipitate formed is suction filtered, washed with ethyl acetate and dried. Yield: 26.1 g (69%, maleate); melting range: 134° C.

### b) 1-{3-[2-(6-benzyloxy-3-oxo-3,4-dihydro-2H-benzo[1,4]oxazin-8-yl)-2-hydroxy-ethylamino]-3-methyl-butyl}-4-ethyl-1,4-dihydro-benzo[1,3]oxazin-2-one

357 mg (1 mmol) 6-benzyloxy-8-(2-ethoxy-2-hydroxy-acetyl)-4H-benzo[1,4]oxazin-3-one and 530 mg (1 mmol) 1-(3-amino-3-methyl-butyl)-4-ethyl-1,4-dihydro-benzo[1,4]oxazine are reacted and worked up analogously to the procedure laid down in 5b). Yield: 308 mg (46%, trifluoroacetate); mass spectroscopy: [M]⁺=560.

### c) 4-ethyl-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[1,3]oxazin-2-one

308 mg (0.46 mmol) 1-{3-[2-(6-benzyloxy-3-oxo-3,4-dihydro-2H-benzo[1,4]oxazin-8-yl)-2-hydroxy-ethylamino]-3-methyl-butyl}-4-ethyl-1,4-dihydro-benzo[1,3]oxazin-2-one are hydrogenated with palladium on charcoal (10%) as catalyst at ambient temperature and 3 bar hydrogen pressure. The catalyst is separated off, the filtrate is concentrated by evaporation and the residue is chromatographed (reverse phase; acetonitrile/water gradient). Yield: 14 mg (5%, trifluoroacetate); mass spectroscopy: [M]⁺=470.

### EXAMPLE 7

### 8-{2-[1,1-dimethyl-3-(2-oxo-3,4-dihydro-2H-quinolin-1-yl)-propylamino]-1-hydroxy-ethyl}-6-hydroxy-4H-benzo[1,4]oxazin-3-one

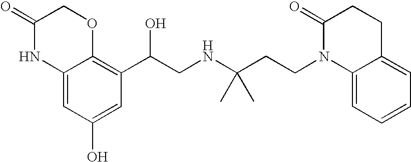

### a) 1-(3-amino-3-methyl-butyl)-3,4-dihydro-quinolin-2-one

This is prepared analogously to the method described for Example 6a) from 15.7 g (107 mmol) 3,4-dihydro-quinolin-2-one and 24.9 g (119 mmol) (3-chloro-1,1-dimethyl-propyl)-(1-phenyl-methylidene)-amine. In a departure from the method mentioned above, the product is precipitated not as a maleate, but as a hydrochloride. Yield: 6.9 g (24%, hydrochloride); melting range: 200-203° C.

### b) 8-{2-[1,1-dimethyl-3-(2-oxo-3,4-dihydro-2H-quinolin-1-yl)-propylamino]-1-hydroxy-ethyl}-6-hydroxy-4H-benzo[1,4]oxazin-3-one

Prepared from 357 mg (1 mmol) 6-benzyloxy-8-(2-ethoxy-2-hydroxy-acetyl)-4H-benzo[1,4]oxazin-3-one and 232 mg (1 mmol) 1-(3-amino-3-methyl-butyl)-3,4-dihydro-quinolin-2-one analogously to the method described for Example 5c). The final purification of the product is carried out by preparative HPLC (Reverse phase, acetonitrile/water gradient with 0.1% trifluoroacetic acid). Yield: 94 mg (17%, trifluoroacetate); mass spectroscopy: [M]⁺=440.

### EXAMPLE 8

### 4,4-diethyl-1-{3-[2-hydroxy-2-(8-hydroxy-2-oxo-1,2-dihydro-quinolin-5-yl)-ethylamino]-3-methyl-butyl}-1,4-dihydro-benzo[1,3]oxazin-2-one

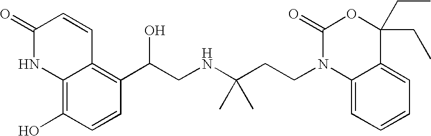

### a) 1-{3-[2-(8-benzyloxy-2-oxo-1,2-dihydro-quinolin-5-yl)-2-hydroxy-ethylamino]-3-methyl-butyl}-4,4-diethyl-1,4-dihydro-benzo[1,3]oxazin-2-one

400 mg (1.4 mmol) 8-benzyloxy-5-oxiranyl-quinolin-2-one and 436 mg (1.5 mmol) 1-(3-amino-3-methyl-butyl)-4,4-diethyl-1,4-dihydro-benzo[1,3]oxazin-2-one in 5 mL n-butanol are stirred for 6 hours at 140° C. The solvent is distilled off and the residue is purified by chromatography (Reverse phase; acetonitrile/water gradient). Beige solid. Yield: 160 mg (20%); mass spectroscopy: [M]⁺=584.

### b) 4,4-diethyl-1-{3-[2-hydroxy-2-(8-hydroxy-2-oxo-1,2-dihydro-quinolin-5-yl)-ethylamino]-3-methyl-butyl}-1,4-dihydro-benzo[1,3]oxazin-2-one

160 mg (0.3 mmol) 1-{3-[2-(8-benzyloxy-2-oxo-1,2-dihydro-quinolin-5-yl)-2-hydroxy-ethylamino]-3-methyl-butyl}-4,4-diethyl-1,4-dihydro-benzo[1,3]oxazin-2-one are dissolved in 5 mL methanol and hydrogenated in the presence of palladium on charcoal (10%). Yield: 49 mg (34%); mass spectroscopy: [M]⁺=494.

### EXAMPLE 9

### 4,4-diethyl-1-{3-[2-hydroxy-2-(6-hydroxy-2,2-dimethyl-3-oxo-3,4-dihydro-2H-benzo[1,4]oxazin-8-yl)-ethylamino]-3-methyl-butyl}-1,4-dihydro-benzo[d][1,3]oxazin-2-one

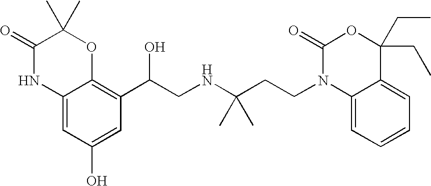

### a) N-(3-acetyl-5-benzyloxy-2-hydroxy-phenyl)-2-bromo-2-methyl-propionamide

4.64 g (25 mmol) 2-bromo-2-methyl-propionyl chloride are added dropwise at 5-20° C. to a solution of 5.15 g (20 mmol) 1-(3-amino-5-benzyloxy-2-hydroxy-phenyl)-ethanone in 20 mL pyridine. After the addition has ended the mixture is stirred for 15 minutes, combined with ice water and 100 mL ethyl acetate and acidified with conc. hydrochloric acid. The organic phase is separated off, washed with water and dried with sodium sulphate. After the solvent has been distilled off the residue is crystallised from a diethyl ether/petroleum ether mixture. Yield: 6.8 g (84%); melting range: 88-90° C.

### b) 8-acetyl-6-benzyloxy-2,2-dimethyl-4H-benzo[1,4]oxazin-3-one

6.60 g (16.2 mmol) N-(3-acetyl-5-benzyloxy-2-hydroxy-phenyl)-2-bromo-2-methyl-propionamide and 2.76 g (20 mmol) potassium carbonate are stirred for 1 hour in 70 mL acetonitrile at reflux temperature. The solid is suction filtered, the filtrate is concentrated by evaporation and the residue is combined with 30 mL ethyl acetate. After further filtration and after distilling off the solvent the crude product is crystallised from a little methanol. Yield: 1.00 g (19%); mass spectroscopy [M+H]⁺=326; melting range=148-150° C.

### c) 6-benzyloxy-8-(2-ethoxy-2-hydroxy-acetyl)-2,2-dimethyl-benzo[1,4]oxazin-3-one

50.12 g (154 mmol) 8-acetyl-6-benzyloxy-2,2-dimethyl-benzo[1,4]oxazin-3-one are reacted with selenium dioxide as oxidising agent and activated charcoal in refluxing dioxane and some water. After cooling the solid is filtered off and washed with dioxane. The filtrate is concentrated by evaporation and the residue is dissolved in 550 mL ethanol and heated for 30 minutes at reflux temperature. It is filtered and the mother liquor is cooled to −18° C., during which time a solid is precipitated which is suction filtered. After recrystallisation from ethanol the product is obtained in the form of a beige solid. Yield: 8.95 g (15%).

### d) 1-{3-[2-(6-benzyloxy-2,2-dimethyl-3-oxo-3,4-dihydro-2H-benzo[1,4]oxazin-8-yl)-2-hydroxy-ethylamino]-3-methyl-butyl}-4,4-diethyl-1,4-dihydro-benzo[d][1,3]oxazin-2-one

Prepared from 406 mg (1 mmol) 6-benzyloxy-8-(2-ethoxy-2-hydroxy-acetyl)-2,2-dimethyl-benzo[1,4]oxazin-3-one and 290 mg (1 mmol) 1-(3-amino-3-methyl-butyl)-4,4-diethyl-1,4-dihydro-benzo[1,3]oxazin-2-one analogously to the method described for Example 5b). The target compound is purified by chromatography on a short column with silica gel (dichloromethane/methanol gradient). White solid. Yield: 145 mg (24%); mass spectroscopy [M+H]⁺=616.

### e) 4,4-diethyl-1-{3-[2-hydroxy-2-(6-hydroxy-2,2-dimethyl-3-oxo-3,4-dihydro-2H-benzo[1,4]oxazin-8-yl)-ethylamino]-3-methyl-butyl}-1,4-dihydro-benzo[d][1,3]oxazin-2-one

130 mg (0.21 mmol) 1-{3-[2-(6-benzyloxy-2,2-dimethyl-3-oxo-3,4-dihydro-2H-benzo[1,4]oxazin-8-yl)-2-hydroxy-ethylamino]-3-methyl-butyl}-4,4-diethyl-1,4-dihydro-benzo[d][1,3]oxazin-2-one are dissolved in 5 mL methanol and hydrogenated in the presence of palladium on charcoal (10%) at ambient temperature. The catalyst is suction filtered, the filtrate is concentrated by evaporation and the residue is purified by chromatography (Reverse phase; acetonitrile/water gradient). White solid. Yield: 41 mg (37%); mass spectroscopy [M+H]⁺=526.

### EXAMPLE 10

### 4,4-diethyl-1-{3-[2-hydroxy-2-(5-hydroxy-2-oxo-2,3-dihydro-benzoxazol-7-yl)-ethylamino]-3-methyl-butyl}-1,4-dihydro-benzo[d][1,3]oxazin-2-one

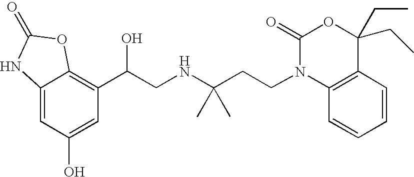

### a) 7-acetyl-5-benzyloxy-3H-benzoxazol-2-one

51.1 mL (97.04 mmol) of a phosgene solution (20 wt. % in toluene) are added at 0° C. to a solution of 22.7 g (88.22 mmol) of 1-(3-amino-5-benzyloxy-2-hydroxy-phenyl)-ethanone in toluene (200 mL). Then 30.7 mL (220.6 mmol) triethylamine are added dropwise such that the temperature does not exceed 5° C. After 1 h stirring at ambient temperature another 4.6 mL phosgene solution and 12 mL triethylamine are added at 0° C. The mixture is stirred for 1 h at ambient temperature, diluted with dichloromethane and combined with saturated aqueous ammonium chloride solution (500 mL) and 2 N aqueous hydrochloric acid (10 mL). After separation of the aqueous phase it is exhaustively extracted with dichloromethane. The combined organic phases are washed with water and saturated aqueous sodium chloride solution, dried with sodium sulphate and concentrated by evaporation i. vac., during which time a beige solid is precipitated. The precipitate is filtered off, washed with a little toluene and dried i. vac. at 50° C. Yield: 18.5 g (74%); R_{f}=0.19 (silica gel, toluene/acetone 95:10); ESI-MS: [M+H]⁺=284.

### b) 5-benzyloxy-7-(2-ethoxy-2-hydroxy-acetyl)-3H-benzoxazol-2-one

14.4 mL (127.1 mmol) HBr (48% in water) are added to a solution of 12.0 g (42.4 mmol) 7-acetyl-5-benzyloxy-3H-benzoxazol-2-one in DMSO (60 mL). The mixture is stirred for 6 h at 60° C. under a gently nitrogen current, poured onto 600 mL ice water and stirred for 20 min. The precipitate formed is filtered off and washed with ice water and cold water/ethyl acetate solution (1:1). The precipitate is dissolved in 300 mL ethanol and 100 mL ethyl acetate and concentrated by evaporation i. vac. The procedure is repeated with 500 mL toluene and then with 500 mL ethanol. The residue is then dissolved in 250 mL ethanol and refluxed for 1 h. After distilling off 30 mL ethanol, the mixture is cooled to ambient temperature and then to 0° C. The precipitate formed is filtered off, washed with 80 mL ice-cold ethanol and 200 mL ether and dried i. vac. at 50° C. Yield: 6.5 g (45%); R_{f}=0.23 (silica gel, dichloromethane/MeOH 25:2); ESI-MS: [M+H—CO₂Et]⁺=270.

### c) 1-{3-[2-(5-benzyloxy-2-oxo-2,3-dihydro-benzoxazol-7-yl)-2-hydroxy-ethylamino]-3-methyl-butyl}-4,4-diethyl-1,4-dihydro-benzo[d][1,3]oxazin-2-one

Obtained from 343 mg (1 mmol) 5-benzyloxy-7-(2-ethoxy-2-hydroxy-acetyl)-3H-benzoxazol-2-one and 290 mg (1 mmol) 1-(3-amino-3-methyl-butyl)-4,4-diethyl-1,4-dihydro-benzo[1,3]oxazin-2-one according to the process described for Example 5b). White solid. Yield: 160 mg (28%); mass spectroscopy [M−H]⁺=572.

### d) 4,4-diethyl-1-{3-[2-hydroxy-2-(5-hydroxy-2-oxo-2,3-dihydro-benzoxazol-7-yl)-ethylamino]-3-methyl-butyl}-1,4-dihydro-benzo[d][1,3]oxazin-2-one

150 mg (0.26 mmol) 1-{3-[2-(5-benzyloxy-2-oxo-2,3-dihydro-benzoxazol-7-yl)-2-hydroxy-ethylamino]-3-methyl-butyl}-4,4-diethyl-1,4-dihydro-benzo[d][1,3]oxazin-2-one are dissolved in 5 mL methanol and hydrogenated with palladium on charcoal as catalyst for 3 hour at ambient temperature. The catalyst is separated off and the filtrate is concentrated by evaporation. Beige solid. Yield: 116 mg (92%); mass spectroscopy [M−H]⁺=484.

HPLC-method (method A): Symmetry C18 (Waters); 3.5 μm; 4.6×150 mm; column temperature: 20° C.; gradient acetonitrile/phosphate buffer (pH 7) 20:80→80:20 in 30 min., flow: 1.0 mL/min; detection at 220 and 254 nm.

### EXAMPLE 11

### 4,4-diethyl-1-{3-[2-hydroxy-2-(7-hydroxy-2-oxo-1,2-dihydro-quinolin-5-yl)-ethylamino]-3-methyl-butyl}-1,4-dihydro-benzo[d][1,3]oxazin-2-one

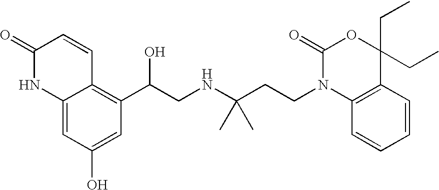

### a) 2-acetyl-4-benzyloxy-6-nitro-phenyl trifluoromethanesulphonate

Triethylamine (92.7 mL, 0.660 mol) is added at −10° C. within 10 min. to a solution of 1-(5-benzyloxy-2-hydroxy-3-nitro-phenyl)-ethanone (90.0 g, 0.313 mol) in abs. dichloromethane (940 mL). A solution of trifluoromethanesulphonic anhydride (65 mL, 0.394 mol) in abs. dichloromethane (40 mL) is added to this red solution within 15 min. and the mixture is stirred for a further 5 min. at −5° C. The brown solution is washed with sat. aqu. ammonium chloride (400 mL) and sat. aqu. NaCl (400 mL) and the phases are separated. Drying with sodium sulphate and evaporation i. vac. yields the crude product as an oil, which solidifies when left to stand. The crude product is dissolved in ether (150 mL), the solution is combined with hexane (800 mL) and the precipitate formed is filtered off. The solid is stirred with ether/hexane (80/20, 100 mL), filtered off and dried in the oven at 40° C. Yield: 118 g (90%); ESI-MS: [M+H]⁺=420.

### b) methyl 3-(2-acetyl-4-benzyloxy-6-nitrophenyl)-acrylate

100 g molecular sieve (4 Å), tris(dibenzylideneacetone)dipalladium (5.88 g, 6.42 mmol), tri-tert-butylphophonium-tetrafluoroborate (3.50 g, 12.06 mmol), dicyclohexylmethylamine (81.2 mL, 0.371 mol), dried tetrabutylammonium iodide (105.8 g, 0.286 mol) and methylacrylate (32.6 mL, 0.362 mol) are added to a solution of 2-acetyl-4-benzyloxy-6-nitro-phenyl trifluoromethanesulphonate (100.0 g, 0.238 mol) in dioxane (360 mL) under a nitrogen atmosphere. The reaction mixture is stirred for 2 hours at 80° C., diluted with ether (2 L) and combined with 500 g silica gel. The suspension is stirred for 10 min., filtered and the silica gel is washed several times with ether (4×600 mL). The combined organic phases are washed with 1 M aqueous hydrochloric acid (300 mL), sodium bicarbonate solution and sodium chloride solution, dried with sodium sulphate and concentrated by evaporation. The oily crude product is recrystallised from hot ethanol (0.75 L). The precipitate is filtered off, washed with ethanol (2×50 mL) and dried at 40° C. Yield: 32.2 g (38%); mass spectroscopy: [M+H]⁺=356.

### c) 5-acetyl-7-benzyloxy-3,4-dihydro-1H-quinolin-2-one

A suspension of methyl 3-(2-acetyl-4-benzyloxy-6-nitrophenyl)-acrylate (5.0 g, 14.07 mmol) in ethanol (100 mL) is hydrogenated with Raney nickel (3 g) at ambient temperature and 4 bar hydrogen pressure. After 6 hours more Raney nickel (2 g) is added and the mixture is hydrogenated for a further 2 hours. The catalyst is separated off and the filtrate is combined with 2 M aqueous hydrochloric acid (15 mL). The product that crystallises out is filtered off and dried. Yield: 1.0 g (24%); mass spectroscopy: [M+H]⁺=296.

### d) 5-acetyl-7-benzyloxy-1H-quinolin-2-one

DDQ (15.0 g, 66.08 mmol) is added to a suspension of 5-acetyl-7-benzyloxy-3,4-dihydro-1H-quinolin-2-one (13.0 g, 44.02 mmol) in dioxane (130 mL) and the mixture is refluxed for 30 minutes. The reaction mixture is cooled to ambient temperature and stirred for a further 2 hours. The precipitate formed is filtered off, washed with dioxane (2×20 mL) and dissolved in dichloromethane/methanol (9:1, 600 mL). The organic phase is washed with sodium bicarbonate solution (2×100 mL), dried with sodium sulphate and concentrated by evaporation. The residue is stirred with methanol, the precipitate formed is filtered off and dried. Yield: 8.3 g (64%); mass spectroscopy: [M+H]⁺=294.

### e) 7-benzyloxy-5-(2-chloracetyl)-1H-quinolin-2-one

5-acetyl-7-benzyloxy-1H-quinolin-2-one (7.0 g, 23.86 mmol) is dissolved in a mixture of 1,2-dichloroethane (147 mL), glacial acetic acid (43 mL) and water (7 mL) and mixed with N-benzyl-trimethylammonium-dichloriodate (19.0 g, 54.58 mmol). The mixture is stirred for 4.5 hours at 65° C., then diluted with sodium bicarbonate solution and 5%. sodium bisulphite solution and stirred for 5 minutes. The precipitate formed is filtered off, washed with water (2×20 mL) and dried in the oven. Yield: 6.0 g (77%); mass spectroscopy: [M+H]⁺=328.

### f) 7-benzyloxy-5-oxiranyl-1H-quinolin-2-one

Lithium borohydride (434 mg, 19.93 mmol) is added at 0-5° C. to a suspension of 7-benzyloxy-5-(2-chloroacetyl)-1H-quinolin-2-one (6 g, 18.31 mmol) in THF (150 mL) and the mixture is stirred for 30 minutes. 2.5 N sodium hydroxide solution (43 mL, 107.50 mmol) is added and the mixture is stirred for 2 hours at 5-10° C. and for 2.5 hours at ambient temperature. Then the reaction mixture is slowly combined with glacial acetic acid (6.5 mL) followed by semisaturated sodium chloride solution (100 mL) and stirred for a further 5 minutes. The precipitate formed is filtered off and the aqueous phase is extracted with ethyl acetate/THF (1/1.5×100 mL). The solid filtered off and the organic phases are combined, dried with sodium sulphate and concentrated by evaporation. The crude product is stirred with methanol (30 mL) and the precipitate is filtered off and dried at ambient temperature. Yield: 4.8 g (89%); mass spectroscopy: [M+H]⁺=294.

### g) 1-{3-[2-(7-benzyloxy-2-oxo-1,2-dihydro-quinolin-5-yl)-2-hydroxy-ethylamino]-3-methyl-butyl}-4,4-diethyl-1,4-dihydro-benzo[d][1,3]oxazin-2-one

A suspension of 7-benzyloxy-5-oxiranyl-1H-quinolin-2-one (112 mg, 0.382 mmol) and 1-(3-amino-3-methyl-butyl)-4,4-diethyl-1,4-dihydro-benzo [d][1,3]oxazin-2-one (220 mg, 0.758 mmol) in isopropanol (1.0 mL) is heated to 135° C. in the microwave for 1 h. The mixture is diluted with EtOAc (10 mL) and washed with 0.5 M aqu. tartaric acid solution, whereupon some of the product is precipitated. The phases are separated and MeOH is added to the aqu. suspension until a clear solution is obtained again. The aqu. phase is extracted with dichloromethane and the combined org. phases are dried on sodium sulphate and concentrated by evaporation i. vac. The residue is stirred with EtOAc and the precipitate is filtered off and dried i. vac. Yield: 152 mg (68%); HPLC-MS: Rₜ=14.8 min. (method A).

### h) 4,4-diethyl-1-{3-[2-hydroxy-2-(7-hydroxy-2-oxo-1,2-dihydro-quinolin-5-yl)-ethylamino]-3-methyl-butyl}-1,4-dihydro-benzo[d][1,3]oxazin-2-one

A suspension of 1-{3-[2-(7-benzyloxy-2-oxo-1,2-dihydro-quinolin-5-yl)-2-hydroxy-ethylamino]-3-methyl-butyl}-4,4-diethyl-1,4-dihydro-benzo[d][1,3]oxazin-2-one (152 mg, 0.338 mmol) and Pd/C (10%) (40 mg) in MeOH (12 mL) is hydrogenated at RT and 1 bar hydrogen pressure for 4 h. The catalyst is filtered through Celite and washed with MeOH (5 mL). The org. phase is concentrated by evaporation, the residue is triturated with EtOAc and the precipitate formed is filtered off and dried i. vac. Yield: 76 mg (46%); R_{f}=0.3 (silica gel, dichloromethane/MeOH/sat. aqu. ammonia 90:10:0.5); ESI-MS: [M+H]⁺=494.

The following synthesis examples require specific starting compounds, the preparation of which is described hereinafter.

### Intermediate Product 1: 1-(3-amino-3-methyl-butyl)-4,4-dipropyl-1,4-dihydro-benzo[d][1,3]oxazin-2-one hydrochloride

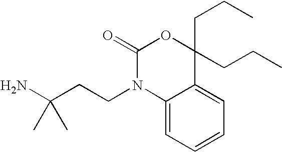

### a) 4-(2-amino-phenyl)-heptan-4-ol

90.0 mL (180.00 mmol) propylmagnesium chloride (2 M in ether) are added dropwise at 0° C. within 30 min. to a solution of 7.00 mL (54.04 mmol) methyl anthranilate in abs. THF (70 mL). The mixture is stirred for 1 h at RT and then combined with 100 mL of 3 M aqu. ammonium chloride solution and EtOAc. The phases are separated and the aqu. phase is exhaustively extracted with EtOAc. The combined org. phases are washed with aqu. KHCO₃ and sat. aqu. NaCl and dried with sodium sulphate. The crude product is used in the next reaction step without any further purification. Yield: 6.70 g (60%).

### b) tert-butyl {3-[2-(1-hydroxy-1-propyl-butyl)-phenylamino]-1,1-dimethyl-propyl}-carbamate

1.40 g (22.27 mmol) sodium cyanoborohydride are added to a solution of 3.10 g (14.05 mmol) 4-(2-amino-phenyl)-heptan-4-ol and 3.60 g (17.88 mmol) tert-butyl (1,1-dimethyl-3-oxo-propyl)-carbamate in MeOH (40 mL) and AcOH (6 mL). The mixture is stirred for 16 h at RT, diluted with EtOAc and washed with 0.5 M aqu. KHSO₄ and sat. aqu. NaCl, dried with sodium sulphate and concentrated by evaporation i. vac. The crude product is used in the next reaction step without any further purification. Yield: 6.00 g (quantitative Yield).

### c) tert-butyl[1,1-dimethyl-3-(2-oxo-4,4-dipropyl-4H-benzo[d][1,3]oxazin-1-yl)-propyl]-carbamate

8.85 mL (16.81 mmol) phosgene solution (20 wt. % in toluene) are slowly added dropwise at 0° C. to a solution of 6.00 g (15.28 mmol) tert-butyl {3-[2-(1-hydroxy-1-propyl-butyl)-phenylamino]-1,1-dimethyl-propyl}-carbamate and 5.32 mL (38.21 mmol) triethylamine in abs. THF (80 mL). The mixture is stirred for 2 h at RT, diluted with EtOAc, mixed with ice and made basic with sat. aqu. ammonia solution. The aqu. phase is exhaustively extracted with EtOAc and the combined org. phases are washed with sat. aqu. NaCl, dried with sodium sulphate and concentrated by evaporation i. vac. After column chromatography (silica gel, cyclohexane/EtOAc 6:1) the product is obtained as a yellow oil. Yield: 4.57 g (71%).

### d) 1-(3-amino-3-methyl-butyl)-4,4-dipropyl-1,4-dihydro-benzo[d][1,3]oxazin-2-one hydrochloride

A solution of 4.20 g (10.03 mmol) tert-butyl[1,1-dimethyl-3-(2-oxo-4,4-dipropyl-4H-benzo[d][1,3]oxazin-1-yl)-propyl]-carbamate in 35 mL formic acid is stirred for 24 h at RT and then poured onto ice. The aqu. phase is made basic with sat. aqu. ammonia solution and exhaustively extracted with EtOAc. The combined org. extracts are washed with sat. aqu. NaCl, dried on sodium sulphate and concentrated by evaporation i. vac. The residue is taken up in EtOAc (50 mL) and combined with 4 mL HCl solution (sat. in EtOAc). The solution is evaporated down and twice mixed with a little EtOH and concentrated by evaporation i. vac. Trituration of the residue with diisopropylether yields the product as a hygroscopic hydrochloride salt. Yield: 2.60 g (73%).

### Intermediate Product 2: 1-(3-amino-3-methyl-butyl)-4,4-diethyl-7-fluoro-1,4-dihydro-benzo[d][1,3]oxazin-2-one

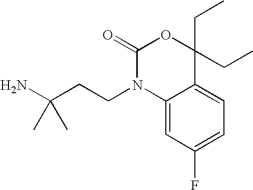

### a) 3-(2-amino-4-fluoro-phenyl)-pentan-3-ol

The product is obtained analogously to intermediate product 1a by reacting methyl 2-amino-4-fluoro-benzoate and ethylmagnesium bromide in dichloromethane at −78° C.→RT. Yield: 4.1 g (99%).

### b) tert-butyl{3-[2-(1-ethyl-1-hydroxy-propyl)-5-fluoro-phenylamino]-1,1-dimethyl-propyl}-carbamate

The product is obtained analogously to intermediate product 1b starting from 3-(2-amino-4-fluoro-phenyl)-pentan-3-ol and tert-butyl (1,1-dimethyl-3-oxo-propyl)-carbamate. The crude product is purified by column chromatography (silica gel, dichloromethane/MeOH 100:0→98:2). Yield: 7.70 g (99%).

### c) tert-butyl[3-(4,4-diethyl-7-fluoro-2-oxo-4H-benzo[d][1,3]oxazin-1-yl)-1,1-dimethyl-propyl]-carbamate

The product is obtained analogously to intermediate product 1c starting from tert-butyl {3-[2-(1-ethyl-1-hydroxy-propyl)-5-fluoro-phenylamino]-1,1-dimethyl-propyl}-carbamate. Yield: 4.20 g (51%).

### d) 1-(3-amino-3-methyl-butyl)-4,4-diethyl-7-fluoro-1,4-dihydro-benzo[d][1,3]oxazin-2-one

The product is prepared analogously to intermediate product 1d starting from tert-butyl[3-(4,4-diethyl-7-fluoro-2-oxo-4H-benzo[d][1,3]oxazin-1-yl)-1,1-dimethyl-propyl]-carbamate as the free base. Yield: 2.90 g (96%); ESI-MS: [M+H]⁺=309.

### Intermediate product 3: 1-(3-amino-3-methyl-butyl)-4,4-diethyl-8-methoxy-1,4-dihydro-benzo[d][1,3]oxazin-2-one

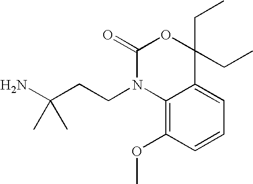

### a) 3-(2-amino-3-methoxy-phenyl)-pentan-3-ol

The product is obtained analogously to intermediate product 1a by reacting methyl 2-amino-3-methoxy-benzoate and ethylmagnesium bromide in dichloromethane at −78° C.→RT. Yield: 5.20 g (92%); HPLC-MS: Rₜ=12.85 min. (method A); ESI-MS: [M+H]⁺=210.

### b) tert-butyl{3-[2-(1-ethyl-1-hydroxy-propyl)-6-methoxy-phenylamino]-1,1-dimethyl-propyl}-carbamate

The product is obtained analogously to intermediate product 1b starting from 3-(2-amino-3-methoxy-phenyl)-pentan-3-ol and tert-butyl (1,1-dimethyl-3-oxo-propyl)-carbamate. The crude product is purified by column chromatography (silica gel, cyclohexane/EtOAc 4:1). Yield: 4.60 g (47%).

### c) tert-butyl[3-(4,4-diethyl-8-methoxy-2-oxo-4H-benzo[d][1,3]oxazin-1-yl)-1,1-dimethyl-propyl]-carbamate

The product is obtained analogously to intermediate product 1c starting from tert-butyl {3-[2-(1-ethyl-1-hydroxy-propyl)-6-methoxy-phenylamino]-1,1-dimethyl-propyl}-carbamate. Yield: 4.60 g (94%).

### d) 1-(3-amino-3-methyl-butyl)-4,4-diethyl-8-methoxy-1,4-dihydro-benzo[d][1,3]oxazin-2-one

The product is obtained analogously to intermediate product 1d starting from tert-butyl[3-(4,4-diethyl-8-methoxy-2-oxo-4H-benzo [d][1,3]oxazin-1-yl)-1,1-dimethyl-propyl]-carbamate as free base. Yield: 3.00 g (93%); ESI-MS: [M+H]⁺=321.

### Intermediate product 4: 1-(3-amino-3-methyl-butyl)-spiro(cyclohexane-1,4′-2H-3′,1′-benzoxazin)-2′-one

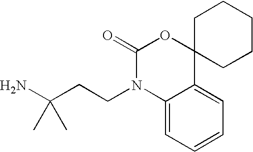

### a) 1-(2-nitro-phenyl)-cyclohexanol

40.16 mL (80.32 mmol) phenylmagnesium chloride (2 M in THF) are added dropwise at −50° C. under a nitrogen atmosphere to a solution of 20.0 g (80.32 mmol) 2-nitro-iodobenzene in abs. THF (150 mL). After 15 min. stirring 9.98 mL (96.30 mmol) cyclohexanone are quickly added. The mixture is heated to RT and stirred for another 2 h. Saturated aqu. ammonium chloride solution is added and the aqu. phase is exhaustively extracted with EtOAc. The combined org. extracts are washed with sat. aqu. NaCl solution, dried with sodium sulphate and concentrated by evaporation i. vac. After column chromatography (silica gel, hexane/EtOAc 20:1) the product is obtained as a brownish oil. Yield: 5.20 g (29%); R_{f}=0.26 (silica gel, hexane/EtOAc 10:1); ESI-MS: [M+H−H₂O]⁺=204.

### b) 1-(2-amino-phenyl)-cyclohexanol

A suspension of 5.20 g (16.45 mmol) 1-(2-nitro-phenyl)-cyclohexanol and 500 mg Raney nickel in EtOH (70 mL) is hydrogenated at RT and 3 bar hydrogen pressure 4 h. The catalyst is filtered through Celite and the filtrate is concentrated by evaporation i. vac. The residue is recrystallised from hexane. Yield: 1.53 g (49%); R_{f}=0.38 (silica gel, hexane/EtOAc 4:1); ESI-MS: [M+H−H₂O]⁺=174.

### c) tert-butyl {3-[2-(1-hydroxy-cyclohexyl)-phenylamino]-1,1-dimethyl-propyl}-carbamate

The product is obtained analogously to intermediate product 1b starting from 1-(2-amino-phenyl)-cyclohexanol and tert-butyl (1,1-dimethyl-3-oxo-propyl)-carbamate. After column chromatography (silica gel, hexane/EtOAc 7:1) the product is obtained as a colourless oil. Yield: 2.65 g (66%); R_{f}=0.50 (silica gel, hexane/EtOAc 4:1).

### d) tert-butyl[3-(spiro(cyclohexane-1,4′-2H-3′,1′-benzoxazin)-2′-oxo-1-yl)-1,1-dimethyl-propyl]-carbamate

The product is obtained analogously to intermediate product 1c starting from tert-butyl {3-[2-(1-hydroxy-cyclohexyl)-phenylamino]-1,1-dimethyl-propyl}-carbamate. Yield: 2.60 g (92%); R_{f}=0.38 (silica gel, hexane/EtOAc 4:1).

### e) 1-(3-amino-3-methyl-butyl)-spiro(cyclohexane-1,4′-2H-3′,1′-benzoxazin)-2′-one

The product is obtained analogously to intermediate product 1d starting from tert-butyl[3-(spiro(cyclohexane-1,4′-2H-3′,1′-benzoxazin)-2′-oxo-1-yl)-1,1-dimethyl-propyl]-carbamate. Yield: 1.80 g (92%); R_{f}=0.10 (silica gel, dichloromethane/MeOH/sat. aqu. ammonia 95:5:0.5); ESI-MS: [M+H]⁺=303.

### EXAMPLE 12

### 1-{3-[2-hydroxy-2-(6-hydroxy-3-oxo-3,4-dihydro-2H-benzo[1,4]oxazin-8-yl)-ethylamino]-3-methyl-butyl}-4,4-dipropyl-1,4-dihydro-benzo[d][1,3]oxazin-2-one

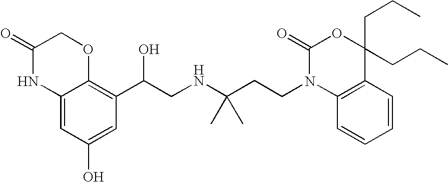

### a) 1-{3-[2-(6-benzyloxy-3-oxo-3,4-dihydro-2H-benzo[1,4]oxazin-8-yl)-2-hydroxy-ethylamino]-3-methyl-butyl}-4,4-dipropyl-1,4-dihydro-benzo[d][1,3]oxazin-2-one

86 μL (0.619 mmol) triethylamine are added at RT under a nitrogen atmosphere to a solution of 200 mg (0.564 mmol) 1-(3-amino-3-methyl-butyl)-4,4-dipropyl-1,4-dihydro-benzo[d][1,3]oxazin-2-one hydrochloride in abs. THF (5 mL) and the mixture is stirred for 30 min. 200 mg (0.560 mmol) 6-benzyloxy-8-(2-ethoxy-2-hydroxy-acetyl)-4H-benzo[1,4]oxazin-3-one are added and the mixture is stirred for a further 2 h at RT. The mixture is cooled to 10° C., combined with 51 mg (2.34 mmol) lithium borohydride, heated to RT and stirred for 1 h at RT. It is cooled to 10° C. again and slowly combined with 15 mL water and 20 mL dichloromethane. The phases are separated and the aqu. phase is extracted with dichloromethane. The combined org. phases are dried with sodium sulphate and concentrated by evaporation i. vac. The residue is dissolved in EtOAc (8 mL) and acidified to pH 2 by the addition of HCl solution (sat. in EtOAc). The precipitate formed is filtered off, washed with EtOAc and dried i. vac. Yield: 270 mg (74%; hydrochloride), HPLC-MS: Rₜ=18.7 min. (method A).

### b) 1-{3-[2-hydroxy-2-(6-hydroxy-3-oxo-3,4-dihydro-2H-benzo[1,4]oxazin-8-yl)-ethylamino]-3-methyl-butyl}-4,4-dipropyl-1,4-dihydro-benzo[d][1,3]oxazin-2-one

A suspension of 270 mg (0.438 mmol) 1-{3-[2-(6-benzyloxy-3-oxo-3,4-dihydro-2H-benzo [1,4]oxazin-8-yl)-2-hydroxy-ethylamino]-3-methyl-butyl}-4,4-dipropyl-1,4-dihydro-benzo[d][1,3]oxazin-2-one and 27 mg Pd/C (10%) in MeOH (8 mL) is hydrogenated at RT and 1 bar hydrogen pressure for 3 h. The catalyst is filtered through Celite and washed with MeOH (5 mL) and the filtrate is evaporated down i. vac. The residue is dissolved in EtOAc/dichloromethane (1:1, 10 mL), acidified to pH 2 by the addition of HCl solution (sat. in EtOAc) and evaporated down i. vac. The residue is triturated with ether, filtered and dried i. vac. Yield: 80 mg (33%; hydrochloride), HPLC-MS: Rₜ=12.8 min. (method A), ESI-MS: [M+H]⁺=526.

### EXAMPLE 13

### 1-{3-[2-hydroxy-2-(5-hydroxy-3-oxo-3,4-dihydro-2H-benzo[1,4]oxazin-8-yl)-ethylamino]-3-methyl-butyl}-4,4-dipropyl-1,4-dihydro-benzo[d][1,3]oxazin-2-one

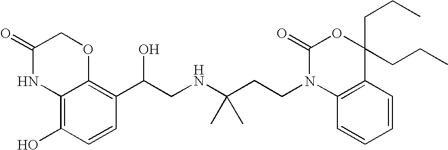

### a) 1-{3-[2-(5-benzyloxy-3-oxo-3,4-dihydro-2H-benzo[1,4]oxazin-8-yl)-2-hydroxy-ethylamino]-3-methyl-butyl}-4,4-dipropyl-1,4-dihydro-benzo[d][1,3]oxazin-2-one

The product is prepared analogously to Example 12a starting from 5-benzyloxy-8-(2,2-dihydroxy-acetyl)-4H-benzo[1,4]oxazin-3-one and 1-(3-amino-3-methyl-butyl)-4,4-dipropyl-1,4-dihydro-benzo[d][1,3]oxazin-2-one hydrochloride. The crude product is dissolved in EtOAc, washed with 5% aqu. NaOH solution and purified by column chromatography (silica gel, dichloromethane/MeOH 98:2→90:10). Yield: 170 mg (49%); HPLC-MS: Rₜ=18.9 min. (method A).

### b) 1-{3-[2-hydroxy-2-(5-hydroxy-3-oxo-3,4-dihydro-2H-benzo[1,4]oxazin-8-yl)-ethylamino]-3-methyl-butyl}-4,4-dipropyl-1,4-dihydro-benzo[d][1,3]oxazin-2-one

The product is prepared analogously to Example 12b starting from 1-{3-[2-(5-benzyloxy-3-oxo-3,4-dihydro-2H-benzo[1,4]oxazin-8-yl)-2-hydroxy-ethylamino]-3-methyl-butyl}-4,4-dipropyl-1,4-dihydro-benzo[d][1,3]oxazin-2-one. Yield: 30 mg (19%, hydrochloride); HPLC-MS: Rₜ=13.0 min. (method A); ESI-MS: [M+H]⁺=526.

### EXAMPLE 14

### 4,4-diethyl-7-fluoro-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[d][1,3]oxazin-2-one

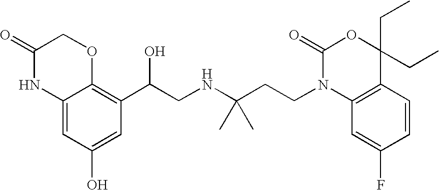

### a) 1-{3-[2-(6-benzyloxy-3-oxo-3,4-dihydro-2H-benzo[1,4]oxazin-8-yl)-2-hydroxy-ethylamino]-3-methyl-butyl}-4,4-diethyl-7-fluoro-1,4-dihydro-benzo[d][1,3]oxazin-2-one

A solution of 232 mg (0.649 mmol) 6-benzyloxy-8-(2-ethoxy-2-hydroxy-acetyl)-4H-benzo[1,4]oxazin-3-one and 200 mg (0.649 mmol) 1-(3-amino-3-methyl-butyl)-4,4-diethyl-7-fluoro-1,4-dihydro-benzo[d][1,3]oxazin-2-one in abs. THF (5 mL) is stirred for 2.5 h at RT. The mixture is cooled to 5° C., combined with 60 mg (2.755 mmol) lithium borohydride, heated to RT and stirred for 1 h. It is cooled again to 5° C. and slowly diluted with 15 ml of water and 20 mL dichloromethane. The phases are separated and the aqu. phase is extracted with dichloromethane. The combined org. phases are dried with sodium sulphate and evaporated down i. vac. The residue is purified by means of column chromatography (silica gel, dichloromethane/MeOH 95:5). Yield: 257 mg (65%); HPLC-MS: Rₜ=16.5 min. (method A).

### b) 4,4-diethyl-7-fluoro-1-{3-[2-hydroxy-2-(6-hydroxy-3-oxo-3,4-dihydro-2H-benzo[1,4]oxazin-8-yl)-ethylamino]-3-methyl-butyl}-1,4-dihydro-benzo[d][1,3]oxazin-2-one

The product is prepared analogously to Example 12b starting from 1-{3-[2-(6-benzyloxy-3-oxo-3,4-dihydro-2H-benzo[1,4]oxazin-8-yl)-2-hydroxy-ethylamino]-3-methyl-butyl}-4,4-diethyl-7-fluoro-1,4-dihydro-benzo[d][1,3]oxazin-2-one. Yield: 170 mg (78%; hydrochloride); HPLC-MS: Rₜ=10.6 min. (method A); ESI-MS: [M+H]⁺=516.

### EXAMPLE 15

### 4,4-diethyl-1-{3-[2-hydroxy-2-(5-hydroxy-3-oxo-3,4-dihydro-2H-benzo[1,4]oxazin-8-yl)-ethylamino]-3-methyl-butyl}-8-methoxy-1,4-dihydro-benzo[d][1,3]oxazin-2-one

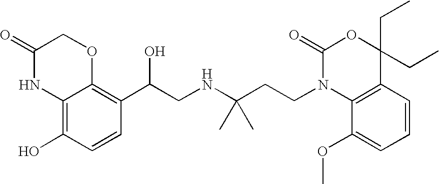

### a) 1-{3-[2-(5-benzyloxy-3-oxo-3,4-dihydro-2H-benzo[1,4]oxazin-8-yl)-2-hydroxy-ethylamino]-3-methyl-butyl}-4,4-diethyl-8-methoxy-1,4-dihydro-benzo[d][1,3]oxazin-2-one

The product is prepared analogously to Example 14a starting from 5-benzyloxy-8-(2,2-dihydroxy-acetyl)-4H-benzo[1,4]oxazin-3-one and 1-(3-amino-3-methyl-butyl)-4,4-diethyl-8-methoxy-1,4-dihydro-benzo[d][1,3]oxazin-2-one. The crude product is purified by column chromatography (silica gel, dichloromethane/MeOH 95:5). Yield: 70 mg (18%); HPLC-MS: Rₜ=16.5 min. (method A).

### b) 4,4-diethyl-1-{3-[2-hydroxy-2-(5-hydroxy-3-oxo-3,4-dihydro-2H-benzo[1,4]oxazin-8-yl)-ethylamino]-3-methyl-butyl}-8-methoxy-1,4-dihydro-benzo[d][1,3]oxazin-2-one

The product is obtained analogously to Example 12b starting from 1-{3-[2-(5-benzyloxy-3-oxo-3,4-dihydro-2H-benzo[1,4]oxazin-8-yl)-2-hydroxy-ethylamino]-3-methyl-butyl}-4,4-diethyl-8-methoxy-1,4-dihydro-benzo[d][1,3]oxazin-2-one. Yield: 40 mg (62%); HPLC-MS: Rₜ=13.3 min. (method A); ESI-MS: [M+H]⁺=528.

### EXAMPLE 16

### 1-(2-{1-[2-hydroxy-2-(5-hydroxy-3-oxo-3,4-dihydro-2H-benzo[1,4]oxazin-8-yl)-ethylamino]-cyclopropyl}-ethyl)-4,4-dimethyl-1,4-dihydro-benzo[d][1,3]oxazin-2-one

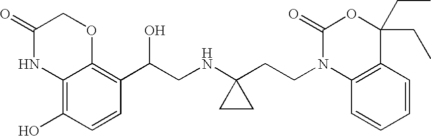

### a) 3-(4,4-dimethyl-2-oxo-4H-benzo[d][1,3]oxazin-1-yl]-propionitrile

10.2 mL (123 mmol) bromopropionitrile are added dropwise to a solution of 20.0 g (112 mmol) 4,4-dimethyl-1,4-dihydro-benzo[d][1,3]oxazin-2-one and 17.4 g (126 mmol) potassium carbonate in 250 mL acetonitrile and the mixture is stirred overnight at reflux temperature. A further 4 mL (48 mmol) bromopropionitrile are added and the mixture is stirred for another 2 hours at reflux temperature. The solid is suction filtered, the filtrate is concentrated by evaporation and the residue is recrystallised from diisopropylether. White solid. Yield: 22.8 g (88%); mass spectroscopy: [M+H]⁺=231.

### b) 1-[2-(1-amino-cyclopropyl)-ethyl]-4,4-dimethyl-1,4-dihydro-benzo[d][1,3]oxazin-2-one

A suspension of 6.0 g (26 mmol) 3-(4,4-dimethyl-2-oxo-4H-benzo[d][1,3]oxazin-1-yl]-propionitrile in 120 mL diethyl ether is combined with 16.5 mL (56 mmol) titanium tetra-isopropoxide while being cooled with an ice bath. Then 18.5 mL of a 3 molar solution of ethylmagnesium bromide in diethyl ether are added dropwise such that the temperature does not exceed 20° C. The mixture is stirred for 30 minutes at ambient temperature and 7.0 mL (55 mmol) boron trifluoride-diethyl ether are added batchwise while cooling with an ice bath. The mixture is stirred for one hour at ambient temperature and 150 mL of 1 molar sodium hydroxide solution are added dropwise while cooling. The reaction mixture is diluted with diethyl ether and the phases are separated. The aqueous phase is extracted with diethyl ether and the combined organic phases are shaken with sodium sulphite solution and repeatedly with 1 molar hydrochloric acid. The hydrochloric acid phases are combined, extracted with diethyl ether, made alkaline with sodium hydroxide solution and exhaustively extracted with dichloromethane. The dichloromethane phases are dried with sodium sulphate and concentrated by evaporation. Light yellow oil. Yield: 1.5 g (22%); mass spectroscopy: [M+H]⁺=261.

### c) 1-(2-{1-[2-(5-benzyloxy-3-oxo-3,4-dihydro-2H-benzo[1,4]oxazin-8-yl)-2-hydroxy-ethylamino]-cyclopropyl}-ethyl)-4,4-dimethyl-1,4-dihydro-benzo[d][1,3]oxazin-2-one

900 mg (2.5 mmol) 5-benzyloxy-8-(2-ethoxy-2-hydroxy-acetyl)-4H-benzo[1,4]oxazin-3-one and 700 mg (2.7 mmol) 1-[2-(1-amino-cyclopropyl)-ethyl]-4,4-dimethyl-1,4-dihydro-benzo[d][1,3]oxazin-2-one are dissolved in 20 mL ethanol and stirred for 30 minutes at 80° C. and another 30 minutes at 50° C. The reaction mixture is cooled, combined with 200 mg (5.3 mmol) sodium borohydride and stirred for 2 hours at ambient temperature. Glacial acetic acid is added, the mixture is stirred for 10 minutes and concentrated by evaporation. The residue is taken up in dichloromethane and washed successively with potassium hydrogen sulphate solution, 15% potassium carbonate solution and sodium hydrogen carbonate solution. Then the organic phase is dried with sodium sulphate and freed from the solvent. The residue is purified by column chromatography (silica gel; ethyl acetate/methanol/ammonia gradient). Recrystallisation from diisopropylether. White solid. Yield: 690 mg (49%); mass spectroscopy: [M+H]⁺=558.

### d) 1-(2-{1-[2-hydroxy-2-(5-hydroxy-3-oxo-3,4-dihydro-2H-benzo[1,4]oxazin-8-yl)-ethylamino]-cyclopropyl}-ethyl)-4,4-dimethyl-1,4-dihydro-benzo[d][1,3]oxazin-2-one

650 mg (1.17 mmol) 1-(2-{1-[2-(5-benzyloxy-3-oxo-3,4-dihydro-2H-benzo[1,4]oxazin-8-yl)-2-hydroxy-ethylamino]-cyclopropyl}-ethyl)-4,4-dimethyl-1,4-dihydro-benzo[d][1,3]oxazin-2-one are dissolved in 30 mL methanol, combined with palladium on charcoal (10%) and hydrogenated at ambient temperature and 3 bar hydrogen pressure. Yield: 240 mg (44%); mass spectroscopy: [M+H]⁺=468.

### EXAMPLE 17

### 1-(2-{1-[2-hydroxy-2-(6-hydroxy-3-oxo-3,4-dihydro-2H-benzo[1,4]oxazin-8-yl)-ethylamino]-cyclopropyl}-ethyl)-4,4-dimethyl-1,4-dihydro-benzo[d][1,3]oxazin-2-one

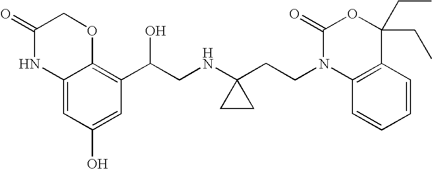

### a) 1-(2-{1-[2-(6-benzyloxy-3-oxo-3,4-dihydro-2H-benzo[1,4]oxazin-8-yl)-2-hydroxy-ethylamino]-cyclopropyl}-ethyl)-4,4-dimethyl-1,4-dihydro-benzo[d][1,3]oxazin-2-one

Prepared from 900 mg (2.5 mmol) 6-benzyloxy-8-(2-ethoxy-2-hydroxy-acetyl)-4H-benzo[1,4]oxazin-3-one and 700 mg (2.7 mmol) 1-[2-(1-amino-cyclopropyl)-ethyl]-4,4-dimethyl-1,4-dihydro-benzo[d][1,3]oxazin-2-one analogously to the method described for Example 16a. White solid. Yield: 630 mg (45%); mass spectroscopy: [M+H]⁺=558.

### b) 1-(2-{1-[2-hydroxy-2-(6-hydroxy-3-oxo-3,4-dihydro-2H-benzo[1,4]oxazin-8-yl)-ethylamino]-cyclopropyl}-ethyl)-4,4-dimethyl-1,4-dihydro-benzo[d][1,3]oxazin-2-o

590 mg (1.06 mmol) 1-(2-{1-[2-(6-benzyloxy-3-oxo-3,4-dihydro-2H-benzo[1,4]oxazin-8-yl)-2-hydroxy-ethylamino]-cyclopropyl}-ethyl)-4,4-dimethyl-1,4-dihydro-benzo[d][1,3]oxazin-2-one are dissolved in 30 mL methanol and hydrogenated in the presence of palladium on charcoal (10%) at ambient temperature and 3 bar hydrogen pressure. Yield: 180 mg (36%); mass spectroscopy: [M+H]⁺=468.

The Examples listed below are obtained analogously to the methods described hereinbefore.

### EXAMPLE 18

### 1-{3-[2-hydroxy-2-(6-hydroxy-3-oxo-3,4-dihydro-2H-benzo[1,4]oxazin-8-yl)-ethylamino]-3-methyl-butyl}-spiro(cyclohexane-1,4′-2H-3′,1′-benzoxazin)-2′-one

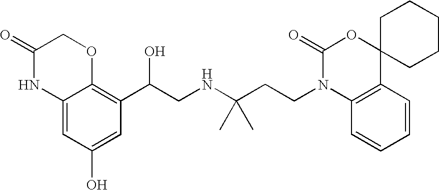

### EXAMPLE 19

### 1-{3-[2-hydroxy-2-(5-hydroxy-3-oxo-3,4-dihydro-2H-benzo[1,4]oxazin-8-yl)-ethylamino]-3-methyl-butyl}-spiro(cyclohexane-1,4′-2H-3′,1′-benzoxazin)-2′-one

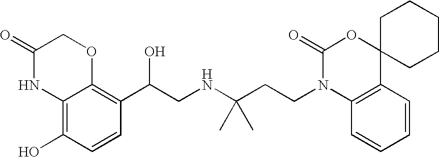

### EXAMPLE 20

### 4,4-dimethyl-1-{3-[2-hydroxy-2-(6-hydroxy-3-oxo-3,4-dihydro-2H-benzo[1,4]oxazin-8-yl)-ethylamino]-propyl}-1,4-dihydro-benzo[d][1,3]oxazin-2-one

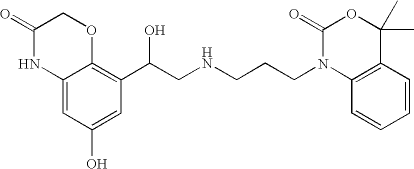

### EXAMPLE 21

### 4,4-dimethyl-1-{3-[2-hydroxy-2-(5-hydroxy-3-oxo-3,4-dihydro-2H-benzo[1,4]oxazin-8-yl)-ethylamino]-propyl}-1,4-dihydro-benzo[d][1,3]oxazin-2-one

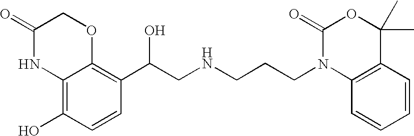

From the compounds mentioned above by way of example, the corresponding enantiomerically pure compounds, i.e. the compounds of formula 1 wherein the asymmetric carbon centre “—CH(OH)—” in the benzyl position to the phenyl ring is in the R configuration may be obtained by methods known in the art.

1) A composition comprising one or more compounds of the formula 1 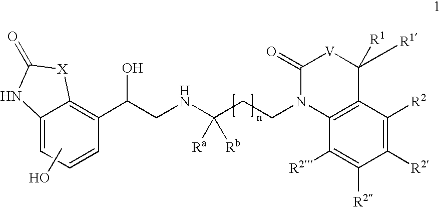
wherein
X denotes a group —O—, —NH—, —CH₂—O—, —CHMe-O—, —C(Me)₂-O—, —CH₂—NH—, —CHMe-NH—, —C(Me)₂-NH—, —CH═CH— or —CH₂—CH₂—;
V denotes a double-bonded group selected from among CH₂, NH and O,
R^{a} and R^{b} which may be identical or different, denote a group selected from among hydrogen, C₁₋₄-alkyl, and halogen-C₁₋₄-alkyl,
or
R^{a} and R^{b} together denote a C₂₋₅-alkylene bridge, wherein one or more hydrogen atoms are optionally be replaced by halogen;
R¹ and R^{1′} which are identical or different, denote a group selected from among hydrogen, C₁₋₆-alkyl, C₃₋₆-cycloalkyl, halogen-C₁₋₆-alkyl, halogen-C₃₋₆-cycloalkyl or C₁₋₆-alkylene-C₃₋₆-cycloalkyl, or
R¹ and R^{1′} together denote a C₂₋₅-alkylene bridge wherein one or more hydrogen atoms are optionally replaced by halogen;
R², R^{2′}, R^{2″} and R^{2′″} which are identical or different, denote a group selected from among hydrogen, C₁₋₆-alkyl, halogen-C₁₋₆-alkylene, OH, HO—C₁₋₆-alkylene, —O—C₁₋₆-alkyl, C₆₋₁₀-aryl, C₆₋₁₀-aryl-C₁₋₄-alkylene, C₆₋₁₀-aryl-C₁₋₆-alkylene-O, COOH, COOC₁₋₆-alkyl, O—C₁₋₆-alkylene-COOH, O—C₁₋₆-alkylene-COOC₁₋₆-alkyl, NHSO₂—C₁₋₆-alkyl, CN, NH₂, NH—C₁₋₆-alkyl, N(C₁₋₆-alkyl)₂, NO₂, S—C₁₋₆-alkyl, SO₂—C₁₋₆-alkyl, SO—C₁₋₆-alkyl, O(CO)C₁₋₆-alkyl, COC₁₋₆-alkyl, NHCOC₁₋₆-alkyl or halogen;
n denotes 0, 1 or 2;
and at least one additional active substance.

2) The composition according to claim 1 further comprising active substance 2 which is one or more compounds selected from the categories of the anticholinergics (2a), PDEIV-inhibitors (2b), steroids (2c), LTD4-antagonists (2d) and EGFR-inhibitors (2e).

3) The composition according to claim 2, wherein
X denotes —O—, —CH₂—O—, —C(Me)₂-O— or —CH═CH—;
V denotes a double-bonded group selected from among CH₂ and O;
R^{a} and R^{b} which are identical or different, denote a group selected from among hydrogen, C₁₋₄-alkyl and fluoro-C₁₋₄-alkyl,
or
R^{a} and R^{b} together denote a group selected from —CH₂—CH₂—, —CH₂—CH₂—CH₂—CH₂— and —CH₂—CH₂—CH₂—CH₂—CH₂—, wherein one or more hydrogen atoms are optionally replaced by fluorine or chlorine;
R¹ and R^{1′} which are identical or different, denote a group selected from among hydrogen, C₁₋₆-alkyl, C₃₋₆-cycloalkyl, halogen-C₁₋₆-alkyl or C₁₋₆-alkylene-C₃₋₆-cycloalkyl,
or
R¹ and R^{1′} together denote a group selected from —CH₂—CH₂, —CH₂—CH₂—CH₂—CH₂— and —CH₂—CH₂—CH₂—CH₂—CH₂, wherein one or more hydrogen atoms are optionally replaced by fluorine or chlorine, preferably fluorine;
R², R^{2′}, R^{2″} and R^{2′″} which are identical or different, denote a group selected from among hydrogen, C₁₋₄-alkyl, CF₃, CHF₂, CH₂F, OH, —O—C₁₋₄-alkyl, phenyl, phenylethyl, benzyl, phenyloxy, benzyloxy, COOH, COOC₁₋₄-alkyl, OCH₂COOH, OCH₂COOC₁₋₄-alkyl, NHSO₂—C₁₋₄-alkyl, fluorine, chlorine or bromine;
n denotes 1.

4) The composition according to claim 3, wherein
X denotes —O—, —CH₂—O—, —C(Me)₂-O— or —CH═CH—;
V O;
R^{a} and R^{b} which are identical or different, denote a group selected from among hydrogen, methyl, ethyl and CF₃, or
R^{a} and R^{b} together denote a group selected from —CH₂—CH₂— and —CH₂—CH₂—CH₂—CH₂—, preferably —CH₂—CH₂—;
R¹ and R^{1′} which are identical or different, denote a group selected from among hydrogen, methyl, ethyl, propyl, cyclopropyl or methylcyclopropyl,
or
R¹ and R^{1′} together denote —CH₂—CH₂—CH₂—CH₂— or —CH₂—CH₂—CH₂—CH₂—CH₂—;
R², R^{2′}, R^{2″} and R^{2′″} which are identical or different, denote a group selected from among hydrogen, methyl, ethyl, propyl, CF₃, CHF₂, CH₂F, OH, methyloxy, ethyloxy, propyloxy, COOH, COOCH₃, COOCH₂CH₃, OCH₂COOH, OCH₂COOCH₃, NHSO₂—CH₃, fluorine, chlorine or bromine.

5) The composition according to claim 1, wherein
R^{a} and R^{b} which are identical or different, denote a group selected from among hydrogen, methyl or ethyl or
R^{a} and R^{b} together denote —CH₂—CH₂—.

6) The composition according to claim 1, which contain one or more compounds of the formula 1 in the form of the acid addition salts with pharmacologically acceptable acids as well as optionally in the form of the solvates and/or hydrates.

7) The composition according to claim 4 wherein the an additional active substance 2 is an anticholinergic (2a).

8) The composition according to claim 7 wherein the anticholinergic (2a) is selected from tiotropium salts (2a.1), oxitropium salts (2a.2), flutropium salts (2a.3), ipratropium salts (2a.4), glycopyrronium salts (2a.5) and trospium salts (2a.6).

9) The composition according to claim 7 wherein the anticholinergic is formula 2a.7 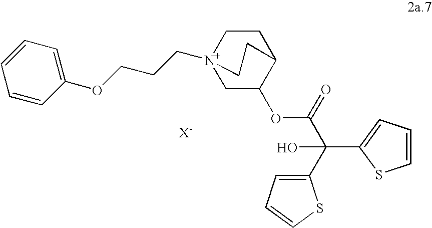
wherein
X⁻ denotes an anion selected from among fluoride, chloride, bromide, iodide, sulphate, phosphate, methanesulphonate, nitrate, maleate, acetate, citrate, fumarate, tartrate, oxalate, succinate, benzoate and p-toluenesulphonate,
optionally in the form of the racemates, enantiomers or hydrates thereof.

10) The composition according to claim 7 the anticholinergic is formula 2a.8 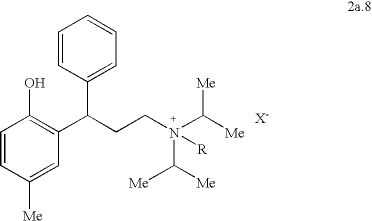
wherein R denotes either methyl (2a.8.1) or ethyl (2a.8.2) and wherein X⁻ selected from fluoride, chloride, bromide, iodide, sulphate, phosphate, methanesulphonate, nitrate, maleate, acetate, citrate, fumarate, tartrate, oxalate, succinate, benzoate and p-toluenesulphonate.

11) The composition according to claim 7 wherein the anticholinergic is formula 2a.9 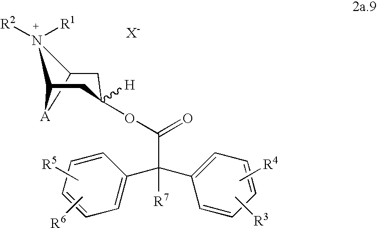
wherein
A denotes a double-bonded group selected from the groups 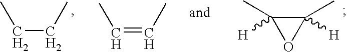
X⁻ is selected from fluoride, chloride, bromide, iodide, sulphate, phosphate, methanesulphonate, nitrate, maleate, acetate, citrate, fumarate, tartrate, oxalate, succinate, benzoate and p-toluenesulphonate;
R¹ and R² which are identical or different denote a group selected from methyl, ethyl, n-propyl and iso-propyl, optionally substituted by hydroxy or fluorine,
R³, R⁴, R⁵ and R⁶, which are identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine,
optionally in the form of the racemates, enantiomers or hydrates thereof.

12) The composition according to claim 7 wherein the anticholinergic is formula 2a.10 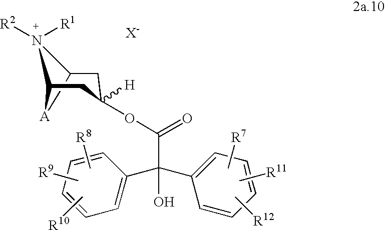
wherein
A, X, R¹ and R² have the meanings given in claim 11 and wherein
R⁷, R⁸, R⁹, R¹⁰, R¹¹ and R¹², which are identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen, optionally in the form of the racemates, enantiomers or hydrates thereof.

13) The composition according to claim 7 wherein the anticholinergic is formula 2a.11 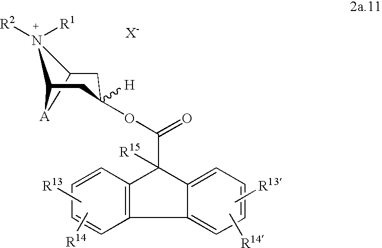
wherein
A and X⁻ have the meanings given in claim 11 and wherein
R¹⁵ denotes hydrogen, hydroxy, methyl, ethyl, —CF₃, CHF₂ or fluorine;
R^{1′} and R^{2′} which are identical or different, denote C₁-C₅-alkyl, which is optionally substituted by C₃-C₆-cycloalkyl, hydroxy or halogen,
or
R^{1′} and R^{2′} together denote a —C₃-C₅-alkylene bridge;
R¹³, R¹⁴, R^{13′} and R^{14′} which are identical or different, denote hydrogen, —C₁-C₄-alkyl, —C₁-C₄-alkyloxy, hydroxy, —CF₃, —CHF₂, CN, NO₂ or halogen,
optionally in the form of the racemates, enantiomers or hydrates thereof.

14) The composition according to claim 7 wherein the anticholinergic is formula 2a.12 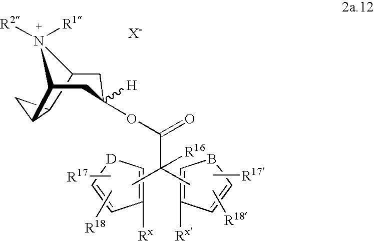
wherein X⁻ has the meanings given in claim 11 and wherein
D and B which are identical or different, denote, O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which are identical or different, denote —C₁-C₅-alkyl, optionally substituted by —C₃-C₆-cycloalkyl, hydroxy or halogen,
or
R^{1″} and R^{2″} together denote a —C₃-C₅-alkylene bridge;
R¹⁷, R¹⁸, R^{17′} and R^{18′}, which are identical or different, denote hydrogen, —C₁-C₄-alkyl, —C₁-C₄-alkyloxy, hydroxy, —CF₃, —CHF₂, CN, NO₂ or halogen;
R^{x} and R^{x′} which are identical or different, denote hydrogen, —C₁-C₄-alkyl, —C₁-C₄-alkyloxy, hydroxy, —CF₃, —CHF₂, CN, NO₂ or halogen,
or
R^{x} and R^{x′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂,
optionally in the form of the racemates, enantiomers or hydrates thereof.

15) The composition according to claim 7 wherein the anticholinergic is formula 2a.13 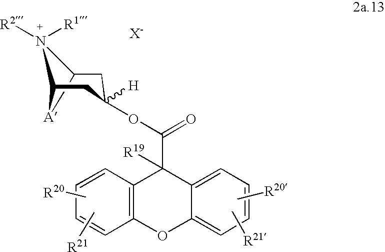
wherein X⁻ has the meanings given in claim 11 and wherein
A′ denotes a double-bonded group selected from 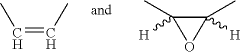
R¹⁹ denotes hydroxy, methyl, hydroxymethyl, ethyl, —CF₃, CHF₂ or fluorine;
R^{1′″} and R^{2′″} which are identical or different, denote C₁-C₅-alkyl, optionally substituted by C₃-C₆-cycloalkyl, hydroxy or halogen,
or
R^{1′″} and R^{2′″} together denote a —C₃-C₅-alkylene bridge;
R²⁰, R²¹, R^{20′} and R^{21′} which are identical or different, denote hydrogen, —C₁-C₄-alkyl, —C₁-C₄-alkyloxy, hydroxy, —CF₃, —CHF₂, CN, NO₂ or halogen,
optionally in the form of the racemates, enantiomers or hydrates thereof.

16) The composition according to claim 4 wherein the additional active substance 2 is a PDE IV inhibitor (2b).

17) The composition according to claim 16, wherein the PDE IV inhibitor 2b is selected from enprofyllin, theophyllin, roflumilast, ariflo (cilomilast), CP-325.366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)ₚ-[(4aR*, 10bS*)-9-ethoxy-1,2,3,4,4a, 10b-hexahydro-8-methoxy-2-methylbenzo[1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-5-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, C1-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

18) The composition according claim 4 wherein the additional active substance 2 is a steroid (2c).

19) The composition according to claim 18, wherein the steroid 2c is selected from prednisolone (2c.1), prednisone (2c.2), butixocortpropionate (2c.3), RPR-106541 (2c.4), flunisolide (2c.5), beclomethasone (2c.6), triamcinolone (2c.7), budesonide (2c.8), fluticasone (2c.9), mometasone (2c.10), ciclesonide (2c.11, rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl)6α,9α-difluoro-11β-hydroxy-16α-methyl-3-oxo-17α-propionyloxy-androsta-1,4-diene-17β-carbothionate (2c.16) and etiprednol-dichloroacetate (2c.17), optionally in the form of the racemates, enantiomers or diastereomers thereof and optionally in the form of the salts and derivatives thereof, the solvates and/or hydrates thereof.

20) The composition according to claim 4 wherein the additional active substance 2 is an LTD4-antagonist (2d).

21) The composition according to claim 20, wherein the LTD4-antagonist 2d is selected from montelukast (2d.1), 1-(((R)-(3-(2-(6,7-difluoro-2-quinolinyl)ethenyl)phenyl)-3-(2-(2-hydroxy-2-propyl)phenyl)thio)methylcyclopropane-acetic acid (2d.2), 1-(((1 (R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropane-acetic acid (2d.3), pranlukast (2d.4), zafirlukast (2d.5), [2-[[2-(4-tert-butyl-2-thiazolyl)-5-benzofuranyl]oxymethyl]phenyl]acetic acid (2d.6), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

22) The composition according to claim 4 wherein the additional active substance 2 is an EGFR-inhibitor (2e).

23) The composition according to claim 22, wherein EGFR-inhibitors 2e are selected from 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidin, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethansulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, cetuximab, trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

24) The composition according to claim 1 further comprising a therapeutically effective amounts of an anticholinergic (2a) as well as therapeutic amounts of a PDEIV-inhibitor (2b), and optionally a pharmaceutically acceptable carrier.

25) The composition according to claim 1 further comprising a therapeutically effective amounts of an anticholinergic (2a), as well as therapeutic amounts of a steroid (2c) and optionally a pharmaceutically acceptable carrier.

26) The composition according to claim 1, further comprising a therapeutically effective amounts of an anticholinergic (2a), as well as therapeutic amounts of an LTD4-antagonist (2d) and optionally a pharmaceutically acceptable carrier.

27) The composition according to claim 1 further comprising a therapeutically effective amounts of an anticholinergic (2a) as well as therapeutic amounts of an EGFR-inhibitor (2e) and optionally a pharmaceutically acceptable carrier.

28) The composition according to claim 1 to 6 further comprising a therapeutically effective amounts of a PDEIV-inhibitor (2b) as well as therapeutic amounts of a steroid (2c) and optionally a pharmaceutically acceptable carrier.

29) The composition according to claim 1 to 6, further comprising a therapeutically effective amounts of a PDEIV-inhibitor (2b) as well as therapeutic amounts of an LTD4-antagonist (2d) and optionally a pharmaceutically acceptable carrier.

30) The composition according to claim 1 further comprising a therapeutically effective amounts of a PDEIV-inhibitor (2b) as well as therapeutic amounts of an EGFR-inhibitor (2e) and optionally a pharmaceutically acceptable carrier.

31) The composition according to claim 1 further comprising a therapeutically effective amounts of a steroid (2c) as well as therapeutic amounts of an LTD4-antagonist (2d) and optionally a pharmaceutically acceptable carrier.

32) The composition according to claim 1 further comprsing therapeutically effective amounts of a steroid (2c) according to one of claims 18 and 19, as well as therapeutic amounts of an EGFR-inhibitor (2e) and optionally a pharmaceutically acceptable carrier.

33) The composition according to claim 1, further comprising a therapeutically effective amounts of an LTD4-antagonist (2d) as well as therapeutic amounts of an EGFR-inhibitor (2e) and optionally a pharmaceutically acceptable carrier.

34) The composition according to claim 11 wherein
R¹ and R² are unsubstituted methyl.

35) The composition according to claim 14 wherein
D and B are identical.

36) The composition according to claim 1 wherein it is in the form of a pharmaceutical formulation suitable for inhalation.

37) The composition according to claim 36, characterised in that it is a preparation selected from inhalable powders, propellant-driven metered-dose aerosols and propellant-free inhalable solutions and suspensions.

38) The composition according to claim 37, wherein the preparation is an inhalable powder which contains 1 and 2 in admixture with suitable physiologically acceptable excipients selected from monosaccharides, disaccharides, oligo- and polysaccharides, polyalcohols and salts, or mixtures of these excipients with one another.

39) The composition according to claim 37, characterised in that the preparation is a propellant-drive inhalable aerosol which contains 1 and 2 in dissolved or dispersed form.

40) The composition according to claim 39, characterised in that the inhalable aerosol contains as the propellant gas selected from n-propane, n-butane, isobutene and chlorinated and/or fluorinated derivatives of methane, ethane, propane, butane, cyclopropane or cyclobutane.

41) The composition according to claim 40, wherein the propellant gas is TG11, TG12, TG134a, TG227 or mixtures thereof.

42) The composition according to claim 37, characterised in that the preparation is a propellant-free inhalable solution or suspension which contains as solvent water, ethanol or a mixture of water and ethanol.

43) A method of treating a disease or condition selected from inflammatory and obstructive respiratory complaints, premature labour in midwifery (tocolysis), atrioventricular block for restoring sinus rhythm in the heart, bradycardic heart rhythm disorders (antiarrhythmic), circulatory shock (vasodilatation and increasing the heart volume) and skin irritations or skin inflammation, comprising administering to a patient a composition according to claim 1.

44) A method of treating a disease or condition selected from obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema comprising administering to a patient a composition according to claim 1.

45) A method of treating a disease or condition selected from bronchial asthma, paediatric asthma, severe asthma, acute asthma attacks, chronic bronchitis and COPD (chronic obstructive pulmonary disease), comprising administering to a patient a composition according to claim 1.

46) A method of treating a disease or condition selected from pulmonary emphysema which has its origins in COPD or α1-proteinase inhibitor deficiency comprising administering to a patient a composition according to claim 1.

47) A method of treating a disease or condition selected from allergic alveolitis, asbestosis, silicosis, lymphangiosis carcinomatosa, bronchoalveolar carcinoma and lymphomas comprising administering to a patient a composition according to claim 1.

48) A method of treating a disease or condition selected from pneumonia caused by infections by viruses, bacteria, fungi, protozoa, helminths or other pathogens, pneumonitis caused by aspiration and left heart insufficiency, radiation-induced pneumonitis or fibrosis, lupus erythematodes, systemic sclerodermy or sarcoidosis, Boeck's disease, idiopathic interstitial pneumonia or idiopathic pulmonary fibrosis (IPF) comprising administering to a patient a composition according to claim 1.

49) A method of treating a disease or condition selected from cystic fibrosis and mucoviscidosis comprising administering to a patient a composition according to claim 1.

50) The method according to claim 44, wherein the type of bronchitis is bronchitis caused by bacterial or viral infection, allergic bronchitis or toxic bronchitis.

51) The method according to claim 44, wherein the type of bronchitis is bronchiectasis.

52) The method according to claim 44 for treating ARDS (adult respiratory distress syndrome).

53) The method according to claim 44 for treating pulmonary oedema.

54) The composition according to claim 41, wherein the propellant gas is TG134a or TG227 or mixtures thereof.

55) The composition according to claim 7 wherein the anticholinergic is tiotropium bromide.
